SEQUENCE LISTING (44 sequences)

SEQ ID NO: 1 - protein, 311 aa, Escherichia coli
MRAQFNPFDLYLGTSAGAQNLSAFICNQPGYARKVIMRYTTKREFFDPLRFVRGGNLIDLDWLVEATASQMPLQMDTAARLFDSGKSFYMCACRQDDYAPNYFLPTKQNWLDVIRASSAIPGFYRSGVSLEGINYLDGGISDAIPVKEAARQGAKTLVVIRTVPSQMYYTPQWFKRMERWLGDSSLQPLVNLVQHHETSYRDIQQFIEKPPGKLRIFEIYPPKPLHSIALGSRIPALREDYKLGRLCGRYFLATVGKLLTEKAPLTRHLVPVVTPESIVIPPAPVANDTLVAEVSDAPQANDPTFNNEDLA

SEQ ID NO: 2 - protein, 132 aa, Escherichia coli
MQTQIKVRGYHLDVYQHVNNARYLEFLEEARWDGLENSDSFQWMTAHNIAFVVVNININYRRPAVLSDLLTITSQLQQLNGKSGILSQVITLEPEGQVVADALITFVCIDLKTQKALALEGELREKLEQMVK

SEQ ID NO: 3 - protein, 277 aa, Escherichia coli [sequence omitted]

SEQ ID NO: 4 - protein, 136 aa, Escherichia coli
MIWKRKITLEALNAMGEGNMVGFLDIRFEHIGDDTLEATMPVDSRTKQPFGLLHGGASVVLAESIGSVAGYLCTEGEQKVVGLEINANHVRSAREGRVRGVCKPLHLGSRHQVWQIEIFDEKGRLCCSSRLTTAIL

SEQ ID NO: 5 - protein, 256 aa, Escherichia coli
MNNIWWQTKGQGNVHLVLLHGWGLNAEVWRCIDEELSSHFTLHLVDLPGFGRSRGFGALSLADMAEAVLQQAPDKAIWLGWSLGGLVASQIALTHPERVQALVTVASSPCFSARDEWPGIKPDVLAGFQQQLSDDFQRTVERFLALQTMGTETARQDARALKKTVLALPMPEVDVLNGGLEILKTVDLRQPLQNVSMPFLRLYGYLDGLVPRKVVPMLDKLWPHSESYIFAKAAHAPFISHPAEFCHLLVALKQRV

SEQ ID NO: 6 - protein, 208 aa, Escherichia coli
MMNFNNVFRWHLPFLFLVLLTFRAAAADTLLILGDSLSAGYRMSASAAWPALLNDKWQSKTSVVNASISGDTSQQGLARLPALLKQHQPRWVLVELGGNDGLRGFQPQQTEQTLRQILQDVKAANAEPLLMQIRLPANYGRRYNEAFSAIYPKLAKEFDVPLLPFFMEEVYLKPQWMQDDGIHPNRDAQPFIADWMAKQLQPLVNHDS

SEQ ID NO: 7 - protein, 539 aa, Escherichia coli
MWNDHDSPEEFNFASDVLDYWAQMEEEGKRGPSPAFWWVNGQGDEIKWSFRKLRDLTCRTANVFEQICGLQQGDHLALILPRVPEWWLVTVGCMRTGIIFMPGTTQLKAKDILYRIQISRAKAIVTTASLVPEVESVASECPDLKTKLVVSDHSHEGWLDFCSLIKSASPDHTCIKSKMKDPMAIFFTSGTTGYPKMAKHNQGLAFRSYIPSCRKLLKLKTSDILWCMSDPGWILATVGCLIEPWTSGCTVFIHHLPQFDPKVIVEVLFKYPITQCLAAPGVYRMVLQQKTSNLRFPTLEHCTTGGESLLPEEYEQWKQRTGLSIHEVYGQSETGISSATLREMKIKRGSIGKAILPFDLQIIDEKGNILPPNTEGYIGIRIKPTRPLGLFMEYENSPESTSEVECGDFYNSGDRATIDEEGYIWFLGRGDDVINASGYRIGPVEVENALAEHPAVAESAVVSSPDKDRGEVVKAFIVLNPEFLSHDQEQLIKELQHHVKSVTAPYKYPRKVEFVSELPKTVTGKIKRKELRNKEFGQL

SEQ ID NO: 8 - protein, 247 aa, Pseudomonas aeruginosa
MSLQGKVALVTGASRGIGQAIALELGRLGAVVIGTATSASGAEKIAETLKANGVEGAGLVLDVSSDESVAATLEHIQQHLGQPLIVVNNAGITRDNLLVRMKDDEWFDVVNTNLNSLYRLSKAVLRGMTKARWGRIINIGSVVGAMGNAGQTNYAAAKAGLEGFTRALAREVGSRAITVNAVAPGFIDTDMTRELPEAQREALLGQIPLGRLGQAEEIAKVVGFLASDGAAYVTGATVPVNGGMYMS

SEQ ID NO: 9 - protein, 247 aa, Pseudomonas aeruginosa
MSLQGKVALVTGASRGIGQAIALELGRLGAVVIGTATSASGAEKIAETLKANGVEGAGLVLDVSSDESVAATLEHIQQHLGQPLIVVNNAGITRDNLLVRMKDDEWFDVVNTNLNSLYRLSKAVLRGMTKARWGRIINIGSVVGAMGNAGQTNYAAAKAGLEGFTRALAREVGSRAITVNAVAPGFIDTDMTRELPEAQREALLAQIPLGRLGQAEEIAKVVGFLASDGAAYVTGATVPVNGGMYMS

SEQ ID NO: 10 - protein, 282 aa, Clostridium beijerinckii
MKKIFVLGAGTMGAGIVQAFAQKGCEVIVRDIKEEFVDRGIAGITKGLEKQVAKGKMSEEDKEAILSRISGTTDMKLAADCDLVVEAAIENMKIKKEIFAELDGICKPEAILASNTSSLSITEVASATKRPDKVIGMHFFNPAPVMKLVEIIKGIATSQETFDAVKELSVAIGKEPVEVAEAPGFVVNGILIPMINEASFILQEGIASVEDIDTAMKYGANHPMGPLALGDLIGLDVCLAIMDVLFTETGDNKYRASSILRKYVRAGWLGRKSGKGFYDYSK

SEQ ID NO: 11 - protein, 261 aa, Clostridium acetobutylicum
MELNNVILEKEGKVAVVTINRPKALNALNSDTLKEMDYVIGEIENDSEVLAVILTGAGEKSFVAGADISEMKEMNTIEGRKFGILGNKVFRRLELLEKPVIAAVNGFALGGGCEIAMSCDIRIASSNARFGQPEVGLGITPGFGGTQRLSRLVGMGMAKQLIFTAQNIKADEALRIGLVNKVVEPSELMNTAKEIANKIVSNAPVAVKLSKQAINRGMQCDIDTALAFESEAFGECFSTEDQKDAMTAFIEKRKIEGFKNR

SEQ ID NO: 12 - protein, 355 aa, Pseudomonas putida
MSDTEVPVLAEVRNRVGHLALNRPVGLNALTLQMIRITWRQLHAWESDPEIVAVVLRANGEKAFCAGGDIRSLYDSYQAGDDLHHVFLEEKYSLDQYIHGYPKPIVALMDGFVLGGGMGLVQGTALRVVTERVKMGMPETSIGYFPDVGGSYFLPRLPGELGLYLGITGIQIRAADALYARLADWCLPSERISEFDRRLDQISWGYAPREILAGLLSSLASNRLLGAELKSLHPAIDEHFTQPDLSAIRASLQAERRPEYQDWAEQTVELLNNRSPLAMSATLKLLRLGRTLSLANCFELELHLERQWFAKGDLIEGVRALLIDKDKTPRWNPPTLEQLDTNRVNEFFDGFQPAT

SEQ ID NO: 13 - protein, 212 aa, Treponema denticola
MIVKPMVRNNICLNAHPQGCKKGVEDQIEYTKKRITAEVKAGAKAPKNVLVLGCSNGYGLASRITAAFGYGAATIGVSFEKAGSETKYGTPGWYNNLAFDEAAKREGLYSVTIDGDAFSDEIKAQVIEEAKKKGIKFDLIVYSLASPVRTDPDTGIMHKSVLKPFGKTFTGKTVDPFTGELKEISAEPANDEEAAATVKVMGGEDWEVGSNS

SEQ ID NO: 14 - protein, 329 aa, Streptomyces sp. strain CL190
MTDVRFRIIGTGAYVPERIVSNDEVGAPAGVDDDWITRKTGIRQRRWAADDQATSDLATAAGRAALKAAGITPEQLTVIAVATSTPDRPQPPTAAYVQHHLGATGTAAFDVNAVCSGTVFALSSVAGTLVYRGGYALVIGADLYSRILNPADRKTVVLFGDGAGAMVLGPTSTGTGPIVRRVALHTFGGLTDLIRVPAGGSRQPLDTDGLDAGLQYFAMDGREVRRFVTEHLPQLIKGFLHEAGVDAADISHFVPHQANGVMLDEVFGELHLPRATMHRTVETYGNTGAASIPITMDAAVRAGSFRPGELVLLAGFGGGMAASFALIEW

SEQ ID NO: 15 - protein, 329 aa, Artificial
  Streptomyces sp. strain CL190
  VARIANT (147)..(147): Residue 147 is Val, Ser, or Thr
MTDVRFRIIGTGAYVPERIVSNDEVGAPAGVDDDWITRKTGIRQRRWAADDQATSDLATAAGRAALKAAGITPEQLTVIAVATSTPDRPQPPTAAYVQHHLGATGTAAFDVNAVCSGTVFALSSVAGTLVYRGGYALVIGADLYSRXLNPADRKTVVLFGDGAGAMVLGPTSTGTGPIVRRVALHTFGGLTDLIRVPAGGSRQPLDTDGLDAGLQYFAMDGREVRRFVTEHLPQLIKGFLHEAGVDAADISHFVPHQANGVMLDEVFGELHLPRATMHRTVETYGNTGAASIPITMDAAVRAGSFRPGELVLLAGFGGGMAAAFALIEW

SEQ ID NO: 16 - protein, 329 aa, Artificial
  Streptomyces sp. strain CL190
  VARIANT (147)..(147): Residue 147 is Val, Ser, or Thr
MTDVRFRIIGTGAYVPERIVSNDEVGAPAGVDDDWITRKTGIRQRRWAADDQATSDLATAAGRAALKAAGITPEQLTVIAVATSTPDRPQPPTAAYVQHLLGATGTAAFDVNAVCSGTVFALSSVAGTLVYRGGYALVIGADLYSRXLNPADRKTVVLFGDGAGAMVLGPTSTGTGPIVRRVALHTFGGLTDLIRVPAGGSRQPLDTDGLDAGLQYVAMDGREVRRFVTEHLPQLIKGFLHEAGVDAADISHFVPHQANGVMLDEVFGELHLPRATMHRTVETYGNTGAASIPITMDAAVRAGSFRPGELVLLAGFGGGMAAAFALIEW

SEQ ID NO: 17 - protein, 329 aa, Artificial
  Streptomyces sp. strain CL190
  VARIANT (100)..(100): Residue 100  is His or Leu
  VARIANT (147)..(147): Residue 147 is Ser, Thr, or Val
  VARIANT (271)..(271): Residue 271 is Phe or Val
MTDVRFRIIGTGAYVPERIVSNDEVGAPAGVDDDWITRKTGIRQRRWAADDQATSDLATAAGRAALKAAGITPEQLTVIAVATSTPDRPQPPTAAYVQHXLGATGTAAFDVNAVCSGTVFALSSVAGTLVYRGGYALVIGADLYSRXLNPADRKTVVLFGDGAGAMVLGPTSTGTGPIVRRVALHTFGGLTDLIRVPAGGSRQPLDTDGLDAGLQYFAMDGREVRRFVTEHLPQLIKGFLHEAGVDAADISHFVPHQANGVMLDEVFGELXLPRATMHRTVETYGNTGAASIPITMDAAVRAGSFRPGELVLLAGFGGGMAAAFALIEW

SEQ ID NO: 18 - protein, 370 aa, Acinetobacter schindleri CIP 107287
MGIRITGTGLFHPTDFITNEELVESLNAYVEQYNLENADKIAAGEIEELRGSSAEFIEKASGIKRRYVAEKTGILDPKRLRPLLHERSNDELSIQAEWGVAAAKQAMENAGVTAEDIDVVILSCSNIQRAYPALAIEIQTALGIQGYAYDMNVACSAATFGIKQAADAIKSGARRVLMVNVEITSGHTDFRSRDCHFIFGDVATASIIEETETKTGFEIEDIELFTQFSNNIRNNFGYLNLSEVDADIDNNRFRQDGRKVFKEVCPLVAKMITKQLEKNQIEPTNVKRFWLHQANVNMNELILKLIVGKEHAKSELVPLILDEFANTSSAGVIIALHRTANEVNDGEYGVLCSFGAGYSVGSILVKKHVA

SEQ ID NO: 19 - protein, 370 aa, Artificial
  Acinetobacter schindleri CIP 107287
MGIRITGTGLFHPTDFITNEELVESLNAYVEQYNLENADKIAAGEIEELRGSSAEFIEKASGIKRRYVAEKTGILDPKRLRPLLHERSNDELSIQAEWGVAAAKQAMENAGVTAEDIDVVILSCSNIQRAYPALAIEIQTALGIQGYAYDMNVACSAATFGIKQAADAIKSGARRVLMVNVEIISGHTDFRSRDCHFIFGDVATASIIEETETKTGFEIEDIELFTQFSNNIRNNLGYLNLSEVDADIDNNRFRQDGRKVFKEVCPLAAKMITKQLEKNQIEPTNVKRFWLHQANANMNELILKLIVGKEHAKSELAPLILDEFANTGSAGVIIALHRTANEVNDGEYGVLCSFGAGYSVGSILVKKHVA

SEQ ID NO: 20 - protein, 370 aa, Artificial
  Acinetobacter schindleri CIP 107287
MGIRITGTGLFHPTDFITNEELVESLNAYVEQYNLENADKIAAGEIEELRGSSAEFIEKASGIKRRYVAEKTGILDPKRLRPLLHERSNDELSIQAEWGVAAAKQAMENAGVTAEDIDVVILSCSNIQRAYPALAIEIQTALGIQGYAYDMNVACSAATFGIKQAADAIKSGARRVLMVNVEIISGHTDFRSRDCHFIFGDVATASIIEETETKTGFEIEDIELFTQFSNNIRNNFGYLNLSEVDADIDNNRFRQDGRKVFKEVCPLAAKMITKQLEKNQIEPTNVKRFWLHQANANMNELILKLIVGKEHAKSELVPLILDEFANTSSAGVIIALHRTANEVNDGEYGVLCSFGAGYSVGSILVKKHVA

SEQ ID NO: 21 - protein, 370 aa, Artificial
  Acinetobacter schindleri CIP 107287
MGIRITGTGLFHPTDFITNEELVESLNAYVEQYNLENADKIAAGEIEELRGSSAEFIEKASGIKRRYVAEKTGILDPKRLRPLLHERSNDELSIQAEWGVAAAKQAMENAGVTAEDIDVVILSCSNIQRAYPALAIEIQTALGIQGYAYDMNVACSAATFGIKQAADAIKSGARRVLMVNVEITSGHTDFRSRDCHFIFGDVATASIIEETETKTGFEIEDIELFTQFSNNIRNNFGYLNLSEVDADIDNNRFRQDGRKVFKEVCPLVAKMITKQLEKNQIEPTNVKRFWLHQANANMNELILKLIVGKEHAKSELVPLILDEFANTSSAGVIIALHRTANEVNDGEYGVLCSFGAGYSVGSILVKKHVA

SEQ ID NO: 22 - protein, 458 aa, Hahella chejuensis
MTPLSPVDQIFLWLEKRQQPMHVGGLHIFSFPDDADAKYMTELAQQLRAYATPQAPFNRRLRQRWGRYYWDTDAQFDLEHHFRHEALPKPGRIRELLAHVSAEHSNLMDRERPMWECHLIEGIRGRRFAVYYKAHHCMLDGVAAMRMCVKSYSFDPTATEMPPIWAISKDVTPARETQAPAAGDLVHSLSQLVEGAGRQLATVPTLIRELGKNLLKARDDSDAGLIFRAPPSILNQRITGSRRFAAQSYALERFKAIGKAFQATVNDVVLAVCGSALRNYLLSRQALPDQPLIAMAPMSIRQDDSDSGNQIAMILANLGTHIADPVRRLELTQASARESKERFRQMTPEEAVNYTALTLAPSGLNLLTGLAPKWQAFNVVISNVPGPNKPLYWNGARLEGMYPVSIPVDYAALNITLVSYRDQLEFGFTACRRTLPSMQRLLDYIEQGIAELEKAAGV

SEQ ID NO: 23 - protein, 647 aa, Artificial
  ARTIFICIAL
MSWIERIKSNITPTRKASIPEGVWTKCDSCGQVLYRAELERNLEVCPKCDHHMRMTARNRLHSLLDEGSLVELGSELEPKDVLKFRDSKKYKDRLASAQKETGEKDALVVMKGTLYGMPVVAAAFEFAFMGGSMGSVVGARFVRAVEQALEDNCPLICFSASGGARMQEALMSLMQMAKTSAALAKMQERGLPYISVLTDPTMGGVSASFAMLGDLNIAEPKALIGFAGPRVIEQTVREKLPPGFQRSEFLIEKGAIDMIVRRPEMRLKLASILAKLMNLPAPNPEAPREGVVVPPVPDQEPEALSGGGGSGGGGSGGGGSGGGGSAAASLNFLDFEQPIAELEAKIDSLTAVSRQDEKLDINIDEEVHRLREKSVELTRKIFADLGAWQIAQLARHPQRPYTLDYVRLAFDEFDELAGDRAYADDKAIVGGIARLDGRPVMIIGHQKGRETKEKIRRNFGMPAPEGYRKALRLMQMAERFKMPIITFIDTPGAYPGVGAEERGQSEAIARNLREMSRLGVPVVCTVIGEGGSGGALAIGVGDKVNMLQYSTYSVISPEGCASILWKSADKAPLAAEAMGIIAPRLKELKLIDSIIPEPLGGAHRNPEAMAASLKAQLLADLADLDVLSTEDLKNRRYQRLMSYGYA

SEQ ID NO: 24 - DNA, 121 bp, Escherichia coli
aaatcagactgaagactttatctctctgtcataaaactgtcatattccttacatataactgtcacctgtttgtcctattttgcttctcgtagccaacaaacaatgctttatgaatcctccc

SEQ ID NO: 25 - DNA, 215 bp, Escherichia coli
gatcttgatatcaaacgaacgttttagcaggactgtcgtcggttgccaaccatctgcgagcaaagcatggcgttttgttgcgcgggatcagcaagcctagcggcagttgtttacgcttttattacagatttaataaattaccacattttaagaatattattaatctgtaatatatctttaacaatctcaggttaaaaactttcctgttttcaacg

SEQ ID NO: 26 - DNA, 61 bp, Escherichia coli
ggtttgaataaatgacaaaaagcaaagcctttgtgccgatgaatctctatactgtttcaca

SEQ ID NO: 27 - protein, 156 aa, Escherichia coli
MDIRKIKKLIELVEESGISELEISEGEESVRISRAAPAASFPVMQQAYAAPMMQQPAQSNAAAPATVPSMEAPAAAEISGHIVRSPMVGTFYRTPSPDAKAFIEVGQKVNVGDTLCIVEAMKMMNQIEADKSGTVKAILVESGQPVEFDEPLVVIE

SEQ ID NO: 28 - protein, 449 aa, Escherichia coli
MLDKIVIANRGEIALRILRACKELGIKTVAVHSSADRDLKHVLLADETVCIGPAPSVKSYLNIPAIISAAEITGAVAIHPGYGFLSENANFAEQVERSGFIFIGPKAETIRLMGDKVSAIAAMKKAGVPCVPGSDGPLGDDMDKNRAIAKRIGYPVIIKASGGGGGRGMRVVRGDAELAQSISMTRAEAKAAFSNDMVYMEKYLENPRHVEIQVLADGQGNAIYLAERDCSMQRRHQKVVEEAPAPGITPELRRYIGERCAKACVDIGYRGAGTFEFLFENGEFYFIEMNTRIQVEHPVTEMITGVDLIKEQLRIAAGQPLSIKQEEVHVRGHAVECRINAEDPNTFLPSPGKITRFHAPGGFGVRWESHIYAGYTVPPYYDSMIGKLICYGENRDVAIARMKNALQELIIDGIKTNVDLQIRIMNDENFQHGGTNIHYLEKKLGLQEK

SEQ ID NO: 29 - DNA, 60 bp, Escherichia coli
gagttaaccacgcggcttgccaacggggtctgaatcgctttttttgtatataatgcgtgt

SEQ ID NO: 30 - protein, 539 aa, Mus musculus; identical to SEQ ID NO: 7

SEQ ID NO: 31 - protein, 320 aa, Artificial
  Clostridiales bacterium 1_7_47_FAA
MTTRIIGTGSYVPEQIVTNNDLAQIVETNDEWIRSRTGIGERRIATTESTSYMAANAAMRALEQSGVKPEEIDLILLGTSSPDYCFPNGACEVQGMIGAVNAACYDISAACTGFVYALNTAHAFISSGIYKTALVIGSDVLSKLIDWTDRGTCVLFGDGAGAVVVKADETGILGINMHSDGTKGNVLTCGSRTNGNFLLGKKPELGYMTMDGQEVFKFAVRKAPECIKQVLDDAGVAAAEVRYFVLHQANYRIIESIAKRLKVSVDCFPVNMEHYGNTSGASVPLLLDEINRKGMLESGDKIVFSGFGAGLTWGATLLEW

SEQ ID NO: 32 - protein, 349 aa, Escherichia coli
MSKIRVLSVDDSALMRQIMTEIINSHSDMEMVATAPDPLVARDLIKKFNPDVLTLDVEMPRMDGLDFLEKLMRLRPMPVVMVSSLTGKGSEVTLRALELGAIDFVTKPQLGIREGMLAYNEMIAEKVRTAAKASLAAHKPLSAPTTLKAGPLLSSEKLIAIGASTGGTEAIRHVLQPLPLSSPALLITQHMPPGFTRSFADRLNKLCQIGVKEAEDGERVLPGHAYIAPGDRHMELSRSGANYQIKIHDGPAVNRHRPSVDVLFHSVAKQAGRNAVGVILTGMGNDGAAGMLAMRQAGAWTLAQNEASCVVFGMPREAINMGGVCEVVDLSQVSQQMLAKISAGQAIRI

SEQ ID NO: 33 - protein, 266 aa, Escherichia coli
MYQVVASDLDGTLLSPDHTLSPYAKETLKLLTARGINFVFATGRHHVDVGQIRDNLEIKSYMITSNGARVHDLDGNLIFAHNLDRDIASDLFGVVNDNPDIITNVYRDDEWFMNRHRPEEMRFFKEAVFQYALYEPGLLEPEGVSKVFFTCDSHEQLLPLEQAINARWGDRVNVSFSTLTCLEVMAGGVSKGHALEAVAKKLGYSLKDCIAFGDGMNDAEMLSMAGKGCIMGSAHQRLKDLHPELEVIGTNADDAVPHYLRKLYLS

SEQ ID NO: 34 - protein, 134 aa, Escherichia coli
VNTTLFRWPVRVYYEDTDAGGVVYHASYVAFYERARTEMLRHHHFSQQALMAERVAFVVRKMTVEYYAPARLDDMLEIQTEITSMRGTSLVFTQRIVNAENTLLNEAEVLVVCVDPLKMKPRALPKSIVAEFKQ

SEQ ID NO: 35 - protein, 183 aa, Escherichia coli
MKLMFASDIHGSLPATERVLELFAQSGAQWLVILGDVLNHGPRNALPEGYAPAKVAERLNEVAHKVIAVRGNCDSEVDQMLLHFPITAPWQQVLLEKQRLFLTHGHLFGPENLPALNQNDVLVYGHTHLPVAEQRGEIFHFNPGSVSIPKGGNPASYGMLDNDVLSVIALNDQSIIAQVAINP

SEQ ID NO: 36 - protein, 278 aa, Escherichia coli
MEMLEEHRCFEGWQQRWRHDSSTLNCPMTFSIFLPPPRDHTPPPVLYWLSGLTCNDENFTTKAGAQRVAAELGIVLVMPDTSPRGEKVANDDGYDLGQGAGFYLNATQPPWATHYRMYDYLRDELPALVQSQFNVSDRCAISGHSMGGHGALIMALKNPGKYTSVSAFAPIVNPCSVPWGIKAFSSYLGEDKNAWLEWDSCALMYASNAQDAIPTLIDQGDNDQFLADQLQPAVLAEAARQKAWPMTLRIQPGYDHSYYFIASFIEDHLRFHAQYLLK

SEQ ID NO: 37 - protein, 329 aa, Escherichia coli
MSQLPGLSRETRESIAMYHLRVPQTEEELERYYQFRWEMLRKPLHQPKGSERDAWDAMAHHQMVVDEQGNLVAVGRLYINADNEASIRFMAVHPDVQDKGLGTLMAMTLESVARQEGVKRVTCSAREDAVEFFAKLGFVNQGEITTPTTTPIRHFLMIKPVATLDDILHRGDWCAQLQQAWYEHIPLSEKMGVRIQQYTGQKFITTMPETGNQNPHHTLFAGSLFSLATLTGWGLIWLMLRERHLGGTIILADAHIRYSKPISGKPHAVADLGALSGDLDRLARGRKARVQMQVEIFGDETPGAVFEGTYIVLPAKPFGPYEEGGNEEE

SEQ ID NO: 38 - protein, 193 aa, Escherichia coli
MSTLLYLHGFNSSPRSAKASLLKNWLAEHHPDVEMIIPQLPPYPSDAAELLESIVLEHGGDSLGIVGSSLGGYYATWLSQCFMLPAVVVNPAVRPFELLTDYLGQNENPYTGQQYVLESRHIYDLKVMQIDPLEAPDLIWLLQQTGDEVLDYRQAVAYYASCRQTVIEGGNHAFTGFEDYFNPIVDFLGLHHL

SEQ ID NO: 39 - protein, 254 aa, Escherichia coli
MKLNIRAQTAQNQHNNSPIVLVHGLFGSLDNLGVLARDLVNDHNIIQVDMRNHGLSPRDPVMNYPAMAQDLVDTLDAQQIDKATFIGHSMGGKAVMALTALASDRIDKLVAIDIAPVDYHVRRHDEIFAAINAVSESDAQTRQQAAAIMRQHLNEEGVIQFLLKSFVDGEWRFNVPVLWDQYPHIVGWEKIPAWDHPALFIPGGNSPYVSEQYRDDLLAQFPQARAHVIAGAGHWVHAEKPDAVLRAIRRYLND

SEQ ID NO: 40 - DNA, 411 bp, Escherichia coli
atgatatggaaacggaaaatcaccctggaagcactgaatgctatgggtgaaggaaacatggtggggttcctggatattcgctttgaacatattggtgatgacacccttgaagcgacaatgccagtagactcgcggacaaagcagcctttcgggttgctgcatggaggagcatccgtggtactggccgaaagtatcggttccgttgccggttatttatgtaccgaaggtgagcaaaaagtggttggtctggaaatcaatgctaaccacgtccgctcggcacgagaagggcgggtgcgcggcgtatgcaaaccgttgcatctcggttcgcgtcaccaggtctggcagattgaaatcttcgatgagaaagggcgtttgtgctgttcgtcacgattgacgaccgccattttgtga

SEQ ID NO: 41 - DNA, 771 bp, Escherichia coli
atgaataacatctggtggcagaccaaaggtcaggggaatgttcatcttgtgctgctgcacggatggggactgaatgccgaagtgtggcgttgcattgacgaggaacttagctcgcattttacgctgcaccttgttgacctgcccggcttcgggcgtagccggggatttggtgcgctgtcacttgctgatatggccgaagccgtgctgcaacaggcacctgataaagccatttggttaggctggagtctgggcgggctggtggcaagccagattgcgttaacccatcccgagcgtgttcaggcgctggtcaccgtggcgtcgtcaccttgttttagtgctcgtgacgagtggccggggataaaaccggacgtgctggcgggatttcagcagcaactcagtgatgattttcagcgtacagtggagcggttcctggcgttacaaaccatggggactgaaacggcgcgccaggatgcgcgggcgttgaagaaaaccgttctggcgttaccgatgccggaggttgacgtgcttaatggcgggctggaaatcctgaaaacggtcgatctccgtcagccgctgcaaaacgtgtccatgccgtttttgcgattgtatggctatctcgacggtctggtgccgcgcaaagtggtgccgatgctggataaactttggcctcacagcgaatcatatatcttcgccaaagcggcccatgcgccatttatttcgcatccggccgagttttgtcacctgctggtggcgttgaagcagagggtgtag

SEQ ID NO: 42 - DNA, 627 bp, Escherichia coli
atgatgaacttcaacaatgttttccgctggcatttgcccttcctgttcctggtcctgttaaccttccgtgccgccgcagcggacacgttattgattctgggtgatagcctgagcgccgggtatcgaatgtctgccagcgcggcctggcctgccttgttgaatgataagtggcagagtaaaacgtcggtagttaatgccagcatcagcggcgacacctcgcaacaaggactggcgcgccttccggctctgctgaaacagcatcagccgcgttgggtgctggttgaactgggcggcaatgacggtttgcgtggttttcagccacagcaaaccgagcaaacgctgcgccagattttgcaggatgtcaaagccgccaacgctgaaccattgttaatgcaaatacgtctgcctgcaaactatggtcgccgttataatgaagcctttagcgccatttaccccaaactcgccaaagagtttgatgttccgctgctgcccttttttatggaagaggtctacctcaagccacaatggatgcaggatgacggtattcatcccaaccgcgacgcccagccgtttattgccgactggatggcgaagcagttgcagcctttagtaaatcatgactcataa

SEQ ID NO: 43 - protein, 226 aa, Escherichia coli
MSLSKQVLPHDVKMRYHMDGCVNGHSFTIEGEGAGKPYEGKKILELRVTKGGPLPFAFDILSSVFTYGNRCFCEYPEDMPDYFKQSLPEGHSWERTLMFEDGGCGTASAHISLDKNCFVHKSTFHGVNFPANGPVMQKKAMNWEPSSELITACDGILKGDVTMFLLLEGGHRLKCQFTTSYKAHKAVKMPPNHIIEHVLVKKEVADGFQIQEHAVAKHFTVDVKET

SEQ ID NO: 44 - DNA, 1620 bp, Mus musculus
atgtggaatgaccatgattcacctgaggagtttaactttgcaagtgatgtcctggactactgggctcaaatggaggaggagggcaagagaggaccaagtccagccttttggtgggtgaatggccaaggagatgaaataaagtggagcttcaggaagctgagggacctcacctgtcgcactgccaacgtctttgagcagatttgtggcctgcagcaaggagatcacctggccttgattctgccccgagtgcccgagtggtggttggtgacagtgggctgcatgcgaacagggatcatcttcatgcctgggactacccaactgaaagccaaggacattctctaccgaatacaaatatctcgagccaaagccattgtgaccacagctagccttgtcccagaggtggaatctgtggcttctgagtgtcctgatctgaaaaccaagctggtggtgtctgatcacagccatgaagggtggcttgatttctgttcactgattaaatcagcatccccagaccatacttgtattaagtcaaagatgaaggatcccatggccatcttcttcaccagtgggaccacaggctaccccaagatggcaaagcacaaccagggacttgccttccggtcatatatcccttcatgcagaaaattattgaagctgaagacatctgacatcttgtggtgcatgtcagacccaggatggattctggctaccgtggggtgcctgatcgagccatggacatcaggatgtacagtcttcatccaccacctccctcaattcgaccccaaagtcattgtagaggtactgttcaaataccccatcactcagtgccttgctgccccaggcgtgtatcgaatggttcttcagcagaaaacctccaacctcaggttccccacccttgagcattgcactactggtggggagagcctgctgcctgaggagtatgagcagtggaagcaaaggacaggtctttccatccacgaggtctatggacagtcagaaacggggatcagcagtgccaccctccgggaaatgaagatcaagcgaggctccatagggaaggccatcttaccctttgacttgcagatcatcgatgaaaagggcaacatcctcccacccaacactgaaggatacattggcatcaggatcaagcccaccaggcctctaggcctcttcatggaatatgagaatagcccagagagcacatctgaagtggagtgtggggacttttacaatagtggggatagagcgaccattgatgaagagggctacatctggttcttgggaaggggcgatgatgtcatcaatgcttccgggtatcgcatcgggcctgtagaggtggagaacgccttggcggagcacccagcagtggcagagtctgcggtggtgagcagcccggacaaggatcgaggagaggtggtgaaggcgtttattgtcctcaacccagagttcctgtcacacgatcaggaacagcttatcaaagagctacagcatcatgtgaagtcagtgacggcaccatacaagtaccccaggaaggtggagtttgtttcagaattgcccaaaactgtcacaggcaaaatcaaaaggaaggaacttcgaaacaaggagtttggtcagctatag

(19) United States
(12) Patent Application Publication
(10) Pub. No.: US 2020/0308613 A1
(43) Pub. Date: Oct. 1, 2020

(54) METHODS AND GENETICALLY MODIFIED CELLS FOR PRODUCTION OF FATTY ACID ESTERS

(71) Applicant: CARGILL, INCORPORATED, WAYZATA, MN (US)

(72) Inventors: Michael Tai Man LOUIE, Camas, WA (US); Ana NEGRETE-RAYMOND, Shorewood, MN (US); Travis Robert WOLTER, Minneapolis, MN (US)

(73) Assignee: CARGILL, INCORPORATED, WAYZATA, MN (US)

(21) Appl. No.: 16/837,248

(22) Filed: Apr. 1, 2020

Related U.S. Application Data
(60) Provisional application No. 62/827,537, filed on Apr. 1, 2019.

(51) Int. Cl. C12P 7/64; C12N 9/02; C12N 9/04; C12N 9/18; C12N 9/12; C12N 9/10

(52) U.S. Cl. CPC C12P 7/6436; C12N 9/001; C12Y 103/01093; C12Y 101/01035; C12N 9/0006; C12Y 203/01041; C12Y 301/01067; C12Y 207/11027; C12N 9/12; C12N 9/1029; C12N 9/18

(57) ABSTRACT

A recombinant cell for producing a fatty acid ester. The recombinant cell is genetically engineered to produce a reduction in free fatty acids compared to a cell that has not been similarly genetically engineered. Methods for producing fatty acid esters while decreasing free fatty acid production are also described.

### BACKGROUND

This disclosure relates to recombinant cells that produce fatty acid chain products. In some aspects, this disclosure relates to recombinant cells that produce C6-C10 fatty acid chain products such as fatty acid esters. In other aspects, this disclosure relates to recombinant cells that produce C6-C10 fatty acid chain products such as fatty acid esters, where the recombinant cells are genetically engineered to produce a reduction in free fatty acids compared to a cell that has not been similarly genetically engineered. Methods for producing fatty acid esters while decreasing free fatty acid production are also described.

Fatty acid chain products such as fatty acid esters have a number of important industrial uses such as solvents for lacquers, paints, varnishes, and other compositions; as plasticizers for organic resins, as fragrances and flavorings, as fuels for jet and other internal combustion engines, and as raw materials for making a variety of downstream products.

While few wild type biological cells naturally produce fatty acids and/or fatty acid chain products of C6-C10 chain length in significant quantities, recombinant cells can be genetically engineered to express heterologous polypeptides for the biosynthesis of C6-C10 fatty acids and/or fatty acid chain products. For example, recombinant cells can be genetically engineered to express heterologous polypeptides for the biosynthesis of C6-C10 fatty acid chain products including fatty acid esters. These genetic modifications can include insertions of heterologous genes and modification and/or deletions of endogenous genes to increase production of C6-C10 chain lengths. Often, heterologous genes can be inserted to provide enzymes to catalyze specific reactions between chemical intermediates along a pathway that results in production of C6-C10 chain lengths. For example, Okamura et al., in PNAS vol. 107, no. 25, pp. 11265-11270 (2010), reported that an enzyme produced by the nphT7 gene of a soil-isolated Streptomyces sp. strain can catalyze a single condensation of acetyl-CoA and malonyl-CoA to produce acetoacetyl-CoA. Additionally, U.S. Application No. 2014/0051136 disclosed that fatty acid chain products can be produced by a cell modified to include the nphT7 gene and additional heterologous genes. WO 2015/010103 described mutants of the nphT7 gene that code for NphT7 enzyme variants that can improve production C6-C10 chain lengths.

Although these recombinant cells can produce C6-C10 fatty acid chain products such as fatty acid esters, there can be limitations to the effectiveness of these recombinant cells to produce higher yields of fatty acid esters. For example, the recombinant cells that produce fatty acid esters can also produce free fatty acids as a side product. In some cases, by producing free fatty acids as a side product, less fatty acid ester is produced by the recombinant cell. In other cases, less fatty acid ester is produced by the recombinant cell because free fatty acids are produced as a side product by degradation of fatty acid esters. Additionally, the presence of free fatty acids can be deleterious to the growth and/or metabolism of the recombinant cells.

### SUMMARY

The present disclosure generally relates to recombinant cells that produce fatty acid esters. One aspect provides a recombinant cell for producing a fatty acid ester, wherein the recombinant cell is genetically engineered for reduced activity of a polypeptide with at least 80%, 85%, 90%, 95%, 98%, or 100% sequence identity to an amino acid sequence set forth in SEQ ID NO: 1-3, wherein the recombinant cell produces a reduction in total free fatty acids compared to a recombinant cell without reduced activity of a polypeptide with at least 80%, 85%, 90%, 95%, 98%, or 100% sequence identity to an amino acid sequence set forth in SEQ ID NO: 1-3. In some aspects, the reduction in free fatty acids comprises a reduction in C10 free fatty acids. In other aspects, the recombinant cell produces a decreased ratio of total free fatty acids to total fatty acid esters compared to a recombinant cell without reduced activity of a polypeptide with at least 80%, 85%, 90%, 95%, 98%, or 100% sequence identity to an amino acid sequence set forth in SEQ ID NO: 1-3. In some aspects, the recombinant cell produces an increase in total fatty acid ester compared to a recombinant cell without reduced activity of a polypeptide with at least 80%, 85%, 90%, 95%, 98%, or 100% sequence identity to an amino acid sequence set forth in SEQ ID NO: 1-3. In other aspects, the increase in fatty acid ester production comprises an increase in fatty acid ester of C10 chain length. In some aspects, the recombinant cell produces a fatty acid ester of C4, C6, C8, and/or C10 chain length. In other aspects, the recombinant cell produces a fatty acid ester of C8 and/or C10 chain length. In some aspects, the alkoxy group of the fatty acid ester is derived from a C1, C2, C3, or C4 monoalcohol. In other aspects, the alkoxy group of the fatty acid ester is derived from a C1 or C2 monoalcohol. In some aspects, the monoalcohol is methanol.

One aspect provides a recombinant cell for producing a fatty acid ester, wherein the recombinant cell is genetically engineered for reduced activity of a polypeptide with at least 80%, 85%, 90%, 95%, 98%, or 100% sequence identity to an amino acid sequence set forth in SEQ ID NO: 4, wherein the recombinant cell produces a reduction in total free fatty acids compared to a recombinant cell without reduced activity of a polypeptide with at least 80%, 85%, 90%, 95%, 98%, or 100% sequence identity to an amino acid sequence set forth in SEQ ID NO: 4. In some aspects, the reduction in free fatty acids comprises a reduction in C8 and/or C10 free fatty acids. In other aspects, the recombinant cell produces a decreased ratio of total free fatty acids to total fatty acid esters compared to a recombinant cell without reduced activity of a polypeptide with at least 80%, 85%, 90%, 95%, 98%, or 100% sequence identity to an amino acid sequence set forth in SEQ ID NO: 4. In some aspects, the recombinant cell produces an increase in fatty acid ester of C8 chain length compared to a recombinant cell without reduced activity of a polypeptide with at least 80%, 85%, 90%, 95%, 98%, or 100% sequence identity to an amino acid sequence set forth in SEQ ID NO: 4. In other aspects, the recombinant cell produces a fatty acid ester of C4, C6, C8, and/or C10 chain length. In some aspects, the recombinant cell produces a fatty acid ester of C8 and/or C10 chain length. In other aspects, the alkoxy group of the fatty acid ester is derived from a C1, C2, C3, or C4 monoalcohol. In some aspects, the alkoxy group of the fatty acid ester is derived from a C1 or C2 monoalcohol. In other aspects, the monoalcohol is methanol.

One aspect provides a recombinant cell for producing a fatty acid ester, wherein the recombinant cell is genetically engineered for reduced activity of a polypeptide with at least 80%, 85%, 90%, 95%, 98%, or 100% sequence identity to an amino acid sequence set forth in SEQ ID NO: 4 and for reduced activity of a polypeptide with at least 80%, 85%, 90%, 95%, 98%, or 100% sequence identity to an amino acid sequence set forth in SEQ ID NO: 5, wherein the recombinant cell produces a reduction in total free fatty acids compared to a recombinant cell without reduced activity of a polypeptide with at least 80%, 85%, 90%, 95%, 98%, or 100% sequence identity to an amino acid sequence set forth in SEQ ID NO: 4 and/or without reduced activity of a polypeptide with at least 80%, 85%, 90%, 95%, 98%, or 100% sequence identity to an amino acid sequence set forth in SEQ ID NO: 5. In some aspects, the recombinant cell is genetically engineered for reduced activity of a polypeptide with at least 80%, 85%, 90%, 95%, 98%, or 100% sequence identity to an amino acid sequence set forth in SEQ ID NO: 6, and wherein the recombinant cell produces a reduction in total free fatty acids compared to a recombinant cell without reduced activity of a polypeptide with at least 80%, 85%, 90%, 95%, 98%, or 100% sequence identity to an amino acid sequence set forth in SEQ ID NO: 6. In other aspects, the reduction in free fatty acids comprises a reduction in C8 and/or C10 free fatty acids. In some aspects, the recombinant cell produces a decreased ratio of total free fatty acids to total fatty acid esters compared to a recombinant cell without reduced activity of a polypeptide with at least 80%, 85%, 90%, 95%, 98%, or 100% sequence identity to an amino acid sequence set forth in SEQ ID NO: 4, without reduced activity of a polypeptide with at least 80%, 85%, 90%, 95%, 98%, or 100% sequence identity to an amino acid sequence set forth in SEQ ID NO: 5. In other aspects, the recombinant cell produces an increase in fatty acid ester of C8 chain length compared to a recombinant cell without reduced activity of a polypeptide with at least 80%, 85%, 90%, 95%, 98%, or 100% sequence identity to an amino acid sequence set forth in SEQ ID NO: 4, without reduced activity of a polypeptide with at least 80%, 85%, 90%, 95%, 98%, or 100% sequence identity to an amino acid sequence set forth in SEQ ID NO: 5, and/or without reduced activity of a polypeptide with at least 80%, 85%, 90%, 95%, 98%, or 100% sequence identity to an amino acid sequence set forth in SEQ ID NO: 6. In some aspects, the recombinant cell produces a fatty acid ester of C4, C6, C8, and/or C10 chain length. In other aspects, the recombinant cell produces a fatty acid ester of C8 and/or C10 chain length. In some aspects, the alkoxy group of the fatty acid ester is derived from a C1, C2, C3, or C4 monoalcohol. In other aspects, the alkoxy group of the fatty acid ester is derived from a C1 or C2 monoalcohol. In some aspects, the monoalcohol is methanol.

One aspect provides a recombinant cell for producing a fatty acid ester, wherein the recombinant cell is genetically engineered for increased activity of a polypeptide with at least 80%, 85%, 90%, 95%, 98%, or 100% sequence identity to an amino acid sequence set forth in SEQ ID NO: 7, wherein the recombinant cell produces a reduction in total free fatty acids compared to a recombinant cell without increased activity of a polypeptide with at least 80%, 85%, 90%, 95%, 98%, or 100% sequence identity to an amino acid sequence set forth in SEQ ID NO: 7. In some aspects, the reduction in free fatty acids comprises a reduction in C8 free fatty acids. In other aspects, the recombinant cell produces a decreased ratio of total free fatty acids to total fatty acid esters compared to a recombinant cell without increased activity of a polypeptide with at least 80%, 85%, 90%, 95%, 98%, or 100% sequence identity to an amino acid sequence set forth in SEQ ID NO: 7. In some aspects, the recombinant cell produces a fatty acid ester of C4, C6, C8, and/or C10 chain length. In other aspects, the recombinant cell produces a fatty acid ester of C8 and/or C10 chain length. In some aspects, the alkoxy group of the fatty acid ester is derived from a C1, C2, C3, or C4 monoalcohol. In other aspects, the alkoxy group of the fatty acid ester is derived from a C1 or C2 monoalcohol. In some aspects, the monoalcohol is methanol.

One aspect provides a recombinant cell for producing a fatty acid ester, wherein the recombinant cell is genetically engineered for increased activity of a polypeptide with at least 80%, 85%, 90%, 95%, 98%, or 100% sequence identity to an amino acid sequence set forth in SEQ ID NO: 7, wherein the recombinant cell is genetically engineered for reduced activity of a polypeptide with at least 80%, 85%, 90%, 95%, 98%, or 100% sequence identity to an amino acid sequence set forth in SEQ ID NO: 4 and for reduced activity of a polypeptide with at least 80%, 85%, 90%, 95%, 98%, or 100% sequence identity to an amino acid sequence set forth in SEQ ID NO: 5, wherein the recombinant cell produces a reduction in total free fatty acids compared to a recombinant cell without reduced activity of a polypeptide with at least 80%, 85%, 90%, 95%, 98%, or 100% sequence identity to an amino acid sequence set forth in SEQ ID NO: 4 and/or without reduced activity of a polypeptide with at least 80%, 85%, 90%, 95%, 98%, or 100% sequence identity to an amino acid sequence set forth in SEQ ID NO: 5. In some aspects, the reduction in free fatty acids comprises a reduction in C8 and/or C10 free fatty acids. In other aspects, the recombinant cell produces increased C8 fatty acid esters compared to a recombinant cell without reduced activity of a polypeptide with at least 80%, 85%, 90%, 95%, 98%, or 100% sequence identity to an amino acid sequence set forth in SEQ ID NO: 4 and/or for reduced activity of a polypeptide with at least 80%, 85%, 90%, 95%, 98%, or 100% sequence identity to an amino acid sequence set forth in SEQ ID NO: 5. In some aspects, the recombinant cell produces a fatty acid ester of C4, C6, C8, and/or C10 chain length. In other aspects, the recombinant cell produces a fatty acid ester of C8 and/or C10 chain length. In some aspects, the alkoxy group of the fatty acid ester is derived from a C1, C2, C3, or C4 monoalcohol. In other aspects, the alkoxy group of the fatty acid ester is derived from a C1 or C2 monoalcohol. In some aspects, the monoalcohol is methanol.

### BRIEF DESCRIPTION OF THE FIGURES

FIGS. 1A, 1B, and 1C show production of C6, C8, C10, C12 fatty acid methyl esters (FAMEs); C6, C8, C10, and C12 free fatty acids (FFAs); and ratios for total FFA/total FAME for strains with a C8-specific Asch variant and deletion of cheB, yigL, ybgC, yjjU, yfcE, yeiG, ybaW, frmB, yiiD, yqiA, and ybfF, respectively, compared to a control strain with no deletion.

FIGS. 2A, 2B, and 2C show production of C6, C8, C10, C12 fatty acid methyl esters (FAMEs); C6, C8, C10, and C12 free fatty acids (FFAs); and ratios for total FFA/total FAME for strains with a C10-specific Asch variant and deletion of cheB, yigL, ybgC, yjjU, yfcE, yeiG, ybaW, frmB, yiiD, yqiA, and ybfF, respectively, compared to a control with no deletion.

FIGS. 3A, 3B, and 3C show production of C6, C8, C10, C12 fatty acid methyl esters (FAMEs); C6, C8, C10, and C12 free fatty acids (FFAs); and ratios for total FFA/total FAME for strains with a C8/C10-specific Asch variant or C10-specific Asch variant and deletion of ydiI, compared to a control with no deletion.

FIGS. 4A, 4B, and 4C show production of C6, C8, C10, C12 fatty acid methyl esters (FAMEs); C6, C8, C10, and C12 free fatty acids (FFAs); and ratios for total FFA/total FAME for strains with a C10-specific Asch variant and deletion of ydiI, bioH, or both ydiI and bioH, compared to a control strain with no deletions.

FIGS. 5A, 5B, and 5C show production of C6, C8, C10, C12 fatty acid methyl esters (FAMEs); C6, C8, C10, and C12 free fatty acids (FFAs); and ratios for total FFA/total FAME for strains with a C8/C10-specific Asch variant and deletion of ydiI, bioH, or both ydiI and bioH, compared to a control strain with no deletions.

FIGS. 6A, and 6B, show production in fermenter of C6, C8, C10, C12 fatty acid methyl esters (FAMEs); and C6, C8, C10, and C12 free fatty acids (FFAs); for a strain with a C8/C10-specific Asch variant and deletion of ydiI, and bioH, compared to a control strain without the ydiI and bioH deletions.

FIG. 6C shows production phase yield (g/g) for a strain with a C8/C10-specific Asch variant and deletion of ydiI, and bioH, compared to a control strain without the ydiI and bioH deletions.

FIG. 6D shows peak instantaneous glucose consumption rate for a strain with a C8/C10-specific Asch variant and deletion of both ydiI and bioH, compared to a control strain without the ydiI and bioH deletions.

FIGS. 7A and 7B show production of C6, C8, C10, C12 fatty acid methyl esters (FAMEs) and C6, C8, C10, and C12 free fatty acids (FFAs) with a C8-specific Asch variant, C8/C10-specific Asch variant or C10-specific Asch variant and deletion of ydiI, bioH, and tesA, compared to a corresponding control strain with no tesA deletion.

FIGS. 8A and 8B show production of C6, C8, C10, C12 fatty acid methyl esters (FAMEs) and C4, C6, C8, C10, and C12 free fatty acids (FFAs) for strains overexpressing acs compared to corresponding control strains with no acs overexpression.

FIGS. 9A and 9B show production of C6, C8, C10, C12 fatty acid methyl esters (FAMEs) and C4, C6, C8, C10, and C12 free fatty acids (FFAs) for strains with a C8-specific Asch variant and with deletion of ydiI and bioH and overexpression of acs compared to a control strain with no acs overexpression.

FIGS. 9C and 9D show production of C6, C8, C10, C12 fatty acid methyl esters (FAMEs) and C4, C6, C8, C10, and C12 free fatty acids (FFAs) for strains with a C10-specific Asch variant and with deletion of ydiI and bioH and overexpression of acs compared to a control strain with no acs overexpression.

FIGS. 10A, and 10B show production of C6, C8, C10, C12 fatty acid methyl esters (FAME) and C6, C8, C10, and C12 free fatty acids (FFA) for strains overexpressing Cbac in various genetic backgrounds comprising of variations in expression of nphT7, ter, and nphT7 (LSVA), and additionally having ΔydiI-ΔbioH compared to a control strain with no ΔydiI-ΔbioH.

### DETAILED DESCRIPTION

Although some wild type biological cells can naturally produce some types of fatty acid chain products, these wild type biological cells tend to produce fatty acids and fatty acid chain products with chain lengths of C12 or higher. Few wild type biological cells naturally produce fatty acids and/or fatty acid chain products of C6-C10 chain length in significant quantities. Recombinant cells can be genetically engineered to express heterologous polypeptides for the biosynthesis of C6-C10 fatty acids and/or fatty acid chain products. For example, recombinant cells can be genetically engineered to express heterologous polypeptides for the biosynthesis of C6-C10 fatty acid chain products including fatty acid esters. Although these recombinant cells can produce C6-C10 fatty acid chain products such as fatty acid esters, there can be limitations to the effectiveness of these recombinant cells to produce higher yields of fatty acid esters. For example, the recombinant cells that produce fatty acid esters can also produce free fatty acids as a side product. In some cases, by producing free fatty acids as a side product, less fatty acid ester is produced by the recombinant cell. In other cases, less fatty acid ester is produced by the recombinant cell because free fatty acids are produced as a side product by degradation of fatty acid esters. Additionally, the presence of free fatty acids can be deleterious to the growth and/or metabolism of the recombinant cells.

Applicants have shown that recombinant cells that are genetically engineered to produce fatty acid esters can also be genetically engineered to produce a reduction in free fatty acids compared to a cell that has not been similarly genetically engineered. These recombinant cells genetically engineered to produce a reduction in free fatty acids can be genetically engineered for reduction in activity of one or more thioesterases. For example, these recombinant cells genetically engineered to produce a reduction in free fatty acids can be genetically engineered for reduction in activity of one or more thioesterases such as YjjU, YbaW, FrmB, TesA and/or YdiI. Also, these recombinant cells genetically engineered to produce a reduction in free fatty acids can also be genetically engineered for reduction in activity of other enzymes such as BioH. Additionally, these recombinant cells genetically engineered to produce a reduction in free fatty acids can also be genetically engineered to increase activity of other enzymes such as acyl CoA synthetase. In some cases, individual genetic modifications can reduce the production of certain chain length free fatty acids. For example, reduction in activity of YdiI enzyme can reduce the production of C8 free fatty acids, while reduction in activity of BioH can reduce the production of C10 free fatty acids. Combinations of genetic modifications can also lead to a surprising and synergistic reduction in free fatty acids. For example, a reduction in activity of YdiI enzyme and BioH enzyme can lead to a reduction in overall free fatty acids including a reduction in both C8 free fatty acids and C10 free fatty acids. Furthermore, an increase in activity of other enzymes such as ACS can reduce the production of free fatty acids by catalyzing the formation of metabolically active CoA thioesters from the free fatty acids. These metabolically active CoA thioesters can then be used by the recombinant cell to generate fatty acid esters. The ACS enzyme favors free fatty acid substrates of C10 or less chain length. A genetic modification to increase the activity of the ACS enzyme can be combined with genetic modifications to reduce activity of other enzymes to lead to surprising and synergistic reduction in free fatty acids. For example, genetic modifications to reduce activity of YdiI and BioH can be combined with a genetic modification to increase activity of ACS enzyme to lead to surprising and synergistic reduction in total free fatty acids, including reduction in C8 free fatty acids and C10 free fatty acids.

As used herein, the singular forms “a,” “an,” and “the” include plural referents unless the context clearly dictates otherwise. For example, reference to “a nucleic acid” means one or more nucleic acids.

Methods well known to those skilled in the art can be used to construct genetic expression constructs and recombinant cells as described. These methods include, but are not limited to, in vitro recombinant DNA techniques, synthetic DNA techniques, in vivo recombination techniques, and polymerase chain reaction (PCR) techniques. For example, techniques as described in Green & Sambrook, 2012, MOLECULAR CLONING: A LABORATORY MANUAL, Fourth Edition, Cold Spring Harbor Laboratory, New York; Ausubel et al., 1989, CURRENT PROTOCOLS IN MOLECULAR BIOLOGY, Greene Publishing Associates and Wiley Interscience, New York, and PCR Protocols: A Guide to Methods and Applications (Innis et al., 1990, Academic Press, San Diego, Calif.) can be used to construct the genetic expression constructs and the recombinant cells described in this application. Additionally, these methods can include CRISPR-assisted genome-editing technology to make insertions, modifications, and/or deletions.

The term “thioesterase” can refer enzymes which belong to the esterase family and which can hydrolyze a thioester bond to generate an acid and thiol, in the presence of water. For example, a thioesterase can hydrolyze the thioester bond of a fatty acid ester to generate a free fatty acid. In some cases, thioesterases (or thiolester hydrolases) are identified as members of E.C.3.1.2.

The term “YjjU” can refer to a polypeptide with at least 80%, 85%, 90%, 95%, 98%, or 100% sequence identity to an amino acid sequence set forth in SEQ ID NO: 1. In some aspects, the term “YjjU” can refer to a polypeptide endogenous to E. coli that is encoded by a gene with at least 80%, 85%, 90%, 95%, 98%, or 100% sequence identity to an amino acid sequence set forth in SEQ ID NO: 1. In other aspects, the term “YjjU” can refer to a polypeptide endogenous to E. coli with hydrolase activity that is encoded by a gene with at least 80%, 85%, 90%, 95%, 98%, or 100% sequence identity to an amino acid sequence set forth in SEQ ID NO: 1.

The term “YbaW” (or FadM) can refer to a polypeptide with at least 80%, 85%, 90%, 95%, 98%, or 100% sequence identity to an amino acid sequence set forth in SEQ ID NO: 2. In some aspects, the term “YbaW” can refer to a polypeptide endogenous to E. coli that is encoded by a gene with at least 80%, 85%, 90%, 95%, 98%, or 100% sequence identity to an amino acid sequence set forth in SEQ ID NO: 2. In other aspects, the term “YbaW” can refer to a polypeptide endogenous to E. coli with hydrolase activity that is encoded by a gene with at least 80%, 85%, 90%, 95%, 98%, or 100% sequence identity to an amino acid sequence set forth in SEQ ID NO: 2.

The term “FrmB” (or YaiM, b0355, or JW0346) can refer to a polypeptide with at least 80%, 85%, 90%, 95%, 98%, or 100% sequence identity to an amino acid sequence set forth in SEQ ID NO: 3. In some aspects, the term “FrmB” can refer to a polypeptide endogenous to E. coli that is encoded by a gene with at least 80%, 85%, 90%, 95%, 98%, or 100% sequence identity to an amino acid sequence set forth in SEQ ID NO: 3. In other aspects, the term “FrmB” can refer to a polypeptide endogenous to E. coli with hydrolase activity that is encoded by a gene with at least 80%, 85%, 90%, 95%, 98%, or 100% sequence identity to an amino acid sequence set forth in SEQ ID NO: 3.

The term “TesA” (or ApeA, PldC, b0494, and/or JW0483) can refer to a polypeptide with at least 80%, 85%, 90%, 95%, 98%, or 100% sequence identity to an amino acid sequence set forth in SEQ ID NO: 6. In some aspects, the term “TesA” can refer to a polypeptide endogenous to E. coli that is encoded by a gene with at least 80%, 85%, 90%, 95%, 98%, or 100% sequence identity to an amino acid sequence set forth in SEQ ID NO: 6. In other aspects, the term “TesA” can refer to a polypeptide endogenous to E. coli with thioesterase activity that is encoded by a gene with at least 80%, 85%, 90%, 95%, 98%, or 100% sequence identity to an amino acid sequence set forth in SEQ ID NO: 6.

The term “YdiI” (or MenI b1686, and/or JW1676) can refer to a polypeptide with at least 80%, 85%, 90%, 95%, 98%, or 100% sequence identity to an amino acid sequence set forth in SEQ ID NO: 4. In some aspects, the term “YdiI” can refer to a polypeptide endogenous to E. coli that is encoded by a gene with at least 80%, 85%, 90%, 95%, 98%, or 100% sequence identity to an amino acid sequence set forth in SEQ ID NO: 4. In other aspects, the term “YdiI” can refer to a polypeptide endogenous to E. coli with hydrolase activity that is encoded by a gene with at least 80%, 85%, 90%, 95%, 98%, or 100% sequence identity to an amino acid sequence set forth in SEQ ID NO: 4.

The term “BioH” can refer to a polypeptide with at least 80%, 85%, 90%, 95%, 98%, or 100% sequence identity to an amino acid sequence set forth in SEQ ID NO: 5. In some aspects, the term “BioH” can refer to a polypeptide endogenous to E. coli that is encoded by a gene with at least 80%, 85%, 90%, 95%, 98%, or 100% sequence identity to an amino acid sequence set forth in SEQ ID NO: 5. In other aspects, the term “BioH” can refer to a polypeptide endogenous to E. coli with hydrolase activity that is encoded by a gene with at least 80%, 85%, 90%, 95%, 98%, or 100% sequence identity to an amino acid sequence set forth in SEQ ID NO: 5. In some cases, BioH can refer to a pimelyl-(acyl-carrier protein) methyl ester hydrolase enzyme involved in biotin synthesis.

The term “ACS” can refer to a polypeptide with at least 80%, 85%, 90%, 95%, 98%, or 100% sequence identity to an amino acid sequence set forth in SEQ ID NO: 7. In some aspects, the term “ACS” can refer to a polypeptide endogenous to eukaryotes that is encoded by a gene with at least 80%, 85%, 90%, 95%, 98%, or 100% sequence identity to an amino acid sequence set forth in SEQ ID NO: 7. In other aspects, the term “ACS” can refer to a polypeptide endogenous to eukaryotes with acyl-CoA synthetase activity that is encoded by a gene with at least 80%, 85%, 90%, 95%, 98%, or 100% sequence identity to an amino acid sequence set forth in SEQ ID NO: 7. In some cases, ACS can refer to an acyl-CoA synthetase long-chain family member 1 enzyme. In other cases, ACS can refer to a acyl-CoA synthetase long-chain family member 1 enzyme from mouse (Mus musculus) (ACSM).

The term “nucleotide” refers to monomer units that make up nucleic acid polymers including DNA, RNA, derivatives thereof, or combinations thereof. The term “oligonucleotide” refers to short chains of DNA or RNA molecules. The term “polynucleotide” refers to longer chains (e.g., 13 or more nucleotide monomers) of DNA or RNA molecules.

Amino acid residues in all amino acid sequences described herein are ordered in the N-terminus to C-terminus direction. “Upstream” means in the direction toward the N-terminus, and “downstream” means toward the C-terminus direction. The “start” of an amino acid sequence is the first amino acid residue in the N-terminus direction. The first amino acid residue (amino acid residue 1) for any sequence or sub-sequence described herein is the amino acid residue at its N-terminus.

A “sub-sequence” is a sequence of amino acid residues contained within a larger amino acid sequence.

Polypeptides refer to polymeric chains of amino acids connected by peptide bonds. In some aspects, polypeptides can fold into unique three dimensional structures that allow the polypeptides to function as enzymes that catalyze distinct biochemical reactions. For example, a folded polypeptide can form a thioesterase enzyme that can hydrolyze the thioester bond of a fatty acid ester to generate a free fatty acid. In some aspects, more than one folded polypeptide can combine into a complex to form an enzyme. For example, in prokaryotes, four folded polypeptides corresponding to the respective gene products of the accA, accB, accC, and accD genes can form an enzyme with acetyl-CoA carboxylase activity.

“Identity” is used herein to indicate the extent to which two (nucleotide or amino acid) sequences have the same residues at the same positions in a pairwise alignment of the sequences. The identity is expressed herein as a % identity as determined using BLAST (National Center for Biological Information (NCBI) Basic Local Alignment Search Tool) version 2.2.31 software (National Center for Biotechnology Information, U.S. National Library of Medicine, Bethesda, Md., USA). Identity between amino acid sequences is determined using protein BLAST with the following parameters: Max target sequences: 100; Short queries: Automatically adjust parameters for short input sequences; Expect threshold: 10; Word size: 6; Max matches in a query range: 0; Matrix: BLOSUM62; Gap Costs: (Existence: 11, Extension: 1); Compositional adjustments: Conditional compositional score matrix adjustment; Filter: none selected; Mask: none selected. Nucleic acid % sequence identity between nucleic acid sequences is determined using standard nucleotide BLAST with the following default parameters: Max target sequences: 100; Short queries: Automatically adjust parameters for short input sequences; Expect threshold: 10; Word size: 28; Max matches in a query range: 0; Match/Mismatch Scores: 1, -2; Gap costs: Linear; Filter: Low complexity regions; Mask: Mask for lookup table only. A sequence having a % identity score of XX % (for example, 80% or 95%) to a reference sequence as determined in this manner is considered to be XX % identical to or, equivalently, have XX % sequence identity to, the reference sequence.

A “recombinant cell” is a cell whose genetic material has been altered by a human using genetic engineering techniques including molecular cloning. Recombinant cells can comprise cells whose genetic material has been altered by the addition of genetic material from a cell of a different organism by genetic engineering. Recombinant cells can also comprise cells whose genetic material has been altered by the deletion of genetic material by genetic engineering. Recombinant cells can also comprise cells altered by the addition of extra copies of genetic material that is normally native to the recombinant cell by genetic engineering.

For purposes of this application, genetic material such as genes, promoters and terminators is “heterologous” if it is (i) non-native to the recombinant cell and/or (ii) is native to the recombinant cell, but is present at a location different than where that genetic material is present in the wild-type cell and/or (iii) is under the regulatory control of a non-native promoter and/or non-native terminator and/or (iv) if it is native to the recombinant cell, but expressed at a different level than the wild-type cell. Extra copies of native genetic material are considered as “heterologous” for purposes of this application, even if such extra copies are present at the same locus as that genetic material is present in the wild-type cell.

A polypeptide (such as a 3-ketoacyl-CoA synthase enzyme) is “heterologous” if it is non-native to a wild-type version of the recombinant cell, if it is native to the recombinant cell, but is expressed by a gene at a location different than where that gene is present in the wild-type version of the recombinant cell, if it is expressed by a gene that is under the regulatory control of a non-native promoter and/or non-native terminator, if extra copies of the encoding gene are present at the same locus where that gene is normally present in the wild-type version of the recombinant cell, and/or if it is native to the recombinant cell but is translated at a different level than in the wild-type cell.

The term “promoter” refers to a region of DNA that initiates transcription and expression of a gene that is operably linked to the promoter. A gene is operably linked to the promoter, for example, when the gene is located downstream of the promoter. Promoters can be native to the recombinant cell or heterologous to the recombinant cell. Promoters can be strong or weak. A strong promoter provides for a high level of gene expression, whereas a weak promoter provides for a low level of gene expression. An inducible promoter is a promoter that provides for the turning on and off of gene expression in response to a specific stimulus, for example, a lowering concentration of phosphate in the cell culture medium. A constitutive promoter is an unregulated promoter that provides for gene expression without the need for separate induction.

The terms “reducing activity”, “reduced activity”, or “reduced activity of a polypeptide” can refer to reducing, attenuating, and/or eliminating the activity of a polypeptide, including a polypeptide that functions as an enzyme. Genetically engineering a recombinant cell for reduced activity of a polypeptide can refer to reducing, attenuating, and/or eliminating the activity of the polypeptide in the recombinant cell. In other words, the recombinant cell can be genetically engineered such that a gene corresponding to the polypeptide with reduced activity is deleted and/or disrupted at the genomic level. In some cases, the recombinant cell can be genetically engineered such that expression of a gene corresponding to the polypeptide with reduced activity is reduced, attenuated, disrupted, and/or eliminated at the expression level. For example, the recombinant cell can be genetically engineered for reduced activity of a polypeptide such that the recombinant cell produces a reduction in total free fatty acids.

The terms “increasing activity”, “increased activity”, or “increase activity of a polypeptide” can refer to increasing, introducing, and/or restoring the activity of a polypeptide, including a polypeptide that functions as an enzyme. Genetically engineering a recombinant cell for increased activity of a polypeptide can refer to increasing, introducing, and/or restoring activity of a polypeptide in the recombinant cell. In some cases, the recombinant cell can be genetically engineered such that a gene corresponding to the polypeptide with increased activity is introduced at the genomic level. In other cases, the gene corresponding to the polypeptide with increased activity is introduced at the genomic level and coupled to an inducible or constitutive promoter. In some cases, the gene corresponding to the polypeptide with increased activity is introduced to the recombinant cell via a plasmid and coupled to an inducible or constitutive promoter. The introduced gene can be an endogenous gene or a heterologous gene. In some aspects, genetically engineering a recombinant cell for increased activity of a polypeptide can refer to increasing activity of the polypeptide by increasing expression of the gene to result in increased synthesis of the polypeptide.

E.C. numbers are established by the Nomenclature Committee of the International Union of Biochemistry and Molecular Biology (NC-IUBMB) (Enzyme Nomenclature 1992 [Academic Press, San Diego, Calif., ISBN 0-12-227164-5 (hardback), 0-12-227165-3 (paperback)] with Supplement 1 (1993), Supplement 2 (1994), Supplement 3 (1995), Supplement 4 (1997) and Supplement 5 (in Eur. J. Biochem. 1994, 223, 1-5; Eur. J. Biochem. 1995, 232, 1-6; Eur. J. Biochem. 1996, 237, 1-5; Eur. J. Biochem. 1997, 250; 1-6, and Eur. J. Biochem. 1999, 264, 610-650). The E.C. numbers referenced herein are derived from the KEGG Ligand database, maintained by the Kyoto Encyclopedia of Genes and Genomics, sponsored in part by the University of Tokyo. Unless otherwise indicated, the E.C. numbers are as provided in the database as of April 2009.

A “CoA” or “CoA-SH”, as described here refers to Coenzyme A. A CoA substrate can refer to a straight chain carbon chain covalently linked to Coenzyme A. For example, C4-CoA refers to a C4 chain covalently linked to Coenzyme A via a thioester bond between the thiol of the Coenzyme A and a terminal carbon of the C4 chain.

A “free fatty acid” (FFA), as described here, comprises a carboxylic acid with an aliphatic chain, the aliphatic chain having at least four carbon atoms.

A “fatty acid chain product” is a compound having a straight carbon chain formed in a series of one or more reactions at the site of the terminal carboxyl group of a fatty acid or thioester bond of a corresponding —CoA compound. A fatty acid chain product can comprise a straight carbon chain with a different end group such as, for example, an ester, an alcohol group, an amino group, an aldehyde group, a ketone, a methyl group, or an alkenyl group. Fatty acid chain product can include fatty acid ester, fatty alcohol, fatty acid amide, fatty acid imide, and fatty acid amine.

A “fatty acid ester” is an ester compound corresponding to the reaction product of a fatty acid and an alcohol (with loss of water). Fatty acid ester can also correspond to the reaction product of a fatty acyl CoA (or a fatty acyl-ACP) and a monohydric alcohol (with loss of a water or a CoA-SH or an ACP-SH). A monohydric alcohol or monoalcohol is an alcohol with only one —OH alcohol functional group. Fatty acid ester can include, for example, fatty acid methyl ester, fatty acid ethyl ester, fatty acid propyl ester, fatty acid butyl ester, fatty acid pentyl ester, and fatty acid hexyl ester. Fatty acid methyl ester (FAME) is an ester compound corresponding to the reaction product of a fatty acid or a fatty acyl-CoA (or fatty acyl-ACP) and methanol.

Chain lengths of free fatty acids and fatty acid chain products are sometimes indicated herein by the shorthand “CX”, wherein X is a number designating the number of carbon atoms. The number of carbon atoms designated in each case represents the carbon length of the straight-chain compound (after removal of CoA or ACP coenzymes) formed by the recombinant cell through one or more iterations of the reaction cycle: acyl-CoA (or acyl-ACP)+malonyl-CoA to form a 3-ketoacyl compound; reduction of the 3-ketoacyl compounds to form a 3-hydroxyacyl compound; dehydration of the 3-hydroxyacyl-CoA to form a 2-enoylacyl compound; and reduction of the 2-enoylacyl compound to the corresponding acyl compound.

Each iteration of this reaction cycle adds two carbon atoms to the starting acyl-CoA or acyl-ACP. The number of carbon atoms does not include additional carbon atoms that may be added during esterification of the fatty acid or fatty acid chain product, such as, for example, carbons added through the monohydric alcohol, during the formation of the ester. Thus, hexanoic acid methyl ester is considered as a “C6” fatty acid ester compound, the carbon of the methyl ester group not being counted.

The “CX” designation can also apply or refer to fatty acid chain products. For example, C4 FAME refers to methyl butanoate, C6 FAME refers to methyl hexanoate, C8 FAME refers to methyl octanoate, C10 FAME refers to methyl decanoate, and C12 FAME refers to methyl dodecanoate. The “CX” designation can also be used for fatty acid intermediates. For example, C2-CoA refers to acetyl-CoA, C4-CoA refers to butyryl-CoA, C6-CoA refers to hexanoyl-CoA, C8-CoA refers to octanoyl-CoA, C10-CoA refers to decanoyl-CoA, and C12-CoA refers to dodecanoyl-CoA.

The term “engineered biosynthetic pathway” refers to a biosynthetic pathway that is at least partially genetically engineered into a recombinant cell and comprises genes encoding enzymes to carry out a sequence of steps to produce a desired product such as a fatty acid ester. In some aspects, one or more of the genes of the engineered biosynthetic pathway does not naturally occur in a wild type version of the recombinant cell. These genes can be introduced into the recombinant cell by using genetic engineering techniques to introduce one or more heterologous genes to encode the enzymes. In some aspects, one or more of the genes of the engineered biosynthetic pathway do not naturally occur in sufficient copy number in a wild-type version of the recombinant cell and additional copies of the genes must be introduced by using genetic engineering techniques to introduce one or more heterologous genes to overexpress the enzymes. In some cases, the engineered biosynthetic pathway also comprises genetic modifications to the recombinant cell to reduce or eliminate competing metabolic pathways and/or to reduce interfering activities such as degradation of desired products or necessary intermediates.

The term “endogenous” gene refers to a gene that is native to a wild type cell and found in the same location and same copy number as in the wild type cell. The term “overexpress” is used to refer to the expression of a heterologous gene in a recombinant cell at levels higher than the level of gene expression in a wild type cell. In some aspects, an endogenous gene is modified to reduce the expression and/or to reduce the activity of the endogenous gene. The terms “deletion,” “deleted,” “knockout,” and “knocked out” can be used interchangeably to refer to an endogenous gene that has been engineered to no longer be expressed in a recombinant cell and/or to refer to an endogenous gene that has been engineered to have reduced activity or to be inactive. In some aspects, a deleted/knocked out gene is an endogenous gene that is deleted to increase production of a desired product, such as a fatty acid ester.

The terms “variant” and “mutant” are used to describe a protein sequence that has been modified at one or more amino acids, compared to the wild type sequence of a particular protein. Mutations to the amino acid residues encoded by the wild-type genes are designated herein by the shorthand designation for the wild-type protein, followed in parentheses by a 3-, 4- or 5 character code consisting of a first letter designating the amino acid residue in the native enzyme, a 1-, 2- or 3-digit number indicating the position of that amino acid residue in the native enzyme, and a final letter designating the amino acid residue in that position in the mutated enzyme. The single-letter designations are IUPAC amino acid abbreviations as reported, for example, at Eur. J. Biochem. 138:9-37 (1984). For example, the designation “Asch(V296A)” indicates that a valine (V) at amino acid residue position 296 in the wild type 3-ketoacyl-CoA synthase enzyme from Acinetobacter schindleri CIP 107287 (Asch enzyme) has been replaced with an alanine (A). The designation “Asch(V296X)” indicates that a valine (V) at amino acid residue position 296 in the wild type 3-ketoacyl-CoA synthase enzyme from Acinetobacter schindleri CIP 107287 (Asch enzyme) is replaced with an amino acid other than valine.

In some aspects, the recombinant cell is a prokaryotic cell. In some aspects, the recombinant cell is a eukaryotic cell.

In some aspects, the recombinant cell is a microorganism, and may be a single-celled microorganism.

The recombinant cell may be a plant cell, including a cell from a plant within any of the Chlorophyta, Charophyta, Marchantiophyta, Anthocerotophyta, Bryophyta, Lycopodiophyta, Pteridophyta, Cycadophyta, Ginkgophyta, Pinophyta, Gnetophyta or Magnoliophyta plants. Such a plant cell may be, for example, a cell from a plant within any of the genera Arabidopsis, Beta, Glycine, Jatropha, Miscanthus, Panicum, Phalaris, Populus, Saccharum, Salix, Simmondsia, and Zea.

The recombinant cell may be a fungi, microalgae, algae or red algae (heterokont) cell. The recombinant cell may be a yeast cell. A yeast or fungus cell may be an oleaginous yeast or fungus, and/or may be a Crabtree negative yeast or fungus.

The term “oleaginous fungi” refers to yeasts or filamentous fungi, which accumulate at least 10%, 12.5%, 15%, 17.5%, preferably at least 20% or even at least 25% (w/w) of their biomass as lipid. They may even accumulate at least 30%, 40%, 50%, 60%, 70%, 80% (w/w) or more of their biomass as lipids. The biomass is usually measured as cell dry weight (CDW).

A “Crabtree-positive” organism is one that is capable of producing ethanol in the presence of oxygen, whereas a “Crabtree-negative” organism is not. A yeast cell having a Crabtree-negative phenotype is any yeast cell that does not exhibit the Crabtree effect. The term “Crabtree-negative” refers to both naturally occurring and genetically modified organisms. Briefly, the Crabtree effect is defined as the inhibition of oxygen consumption by a microorganism when cultured under aerobic conditions due to the presence of a high concentration of glucose (e.g., 10 g-glucose L-1). In other words, a yeast cell having a Crabtree-positive phenotype continues to ferment irrespective of oxygen availability due to the presence of glucose, while a yeast cell having a Crabtree-negative phenotype does not exhibit glucose mediated inhibition of oxygen consumption. Crabtree-positive yeast produce an excess of alcohol rather than biomass production.

Examples of suitable yeast cells include, Pichia, Candida, Klebsiella, Hansenula, Kluyveromyces, Trichosporon, Brettanomyces, Pachysolen, Issatchenkia, Yamadazyma Saccharomyces, Schizosaccharomyces, Zygosaccharomyces, Debaryomyces, Cryptoococcus, Rhodotorula, Rhodosporidium, Lipomyces and Yarrowia. Examples of specific yeast cells include C. sonorensis, K. marxianus, K. thermotolerans, C. methanesorbosa, Saccharomyces bulderi (S. bulderi), I. orientalis, C. lambica, C. sorboxylosa, C. zemplinina, C. geochares, P. membranifaciens, Z. kombuchaensis, C. sorbosivorans, C. vanderwaltii, C. sorbophila, Z. bisporus, Z. lentus, Saccharomyces bayanus (S. bayanus), D. castellii, C. boidinii, C. etchellsii, K. lactis, P. jadinii, P. anomala, Saccharomyces cerevisiae (S. cerevisiae) Pichia galeiformis, Pichia sp. YB-4149 (NRRL designation), Candida ethanolica, P. deserticola, P. membranifaciens, P. fermentans, Rhodosporidium toruloide, Lipomyces starkeyii, L. lipoferus, Candida revkaufi, C. pulcherrima, C. tropicalis, C. utilis, Trichosporon pullas, T. cutaneum, Rhodotorula glutinous, R. garminis, Yarrowia lipolytica and Saccharomycopsis crataegensis (S. crataegensis). Suitable strains of K. marxianus and C. sonorensis include those described in WO 00/71738 A1, WO 02/42471 A2, WO 03/049525 A2, WO 03/102152 A2 and WO 03/102201A2. Suitable strains of I. orientalis are ATCC strain 32196 and ATCC strain PTA-6648.

In some aspects, the recombinant cell is a bacteria cell. The bacteria may be a gram-positive or gram-negative bacteria. It may be a cell within any of the Chlamydiae, green nonsulfur, actinobacteria, planctomycetes, spirochaetes, fusobacteria, cyanobacteria, thermophilic sulphate-reducer, acidobacteria or proteobacteria classifications of bacteria (Ciccarelli et al., Science 311 (5765): 1283-7 (2006).

Examples of suitable bacteria cells include, for example, those within any of the genera Clostridium, Zymomonas, Escherichia, Salmonella, Rhodococcus, Pseudomonas, Streptomyces, Bacillus, Lactobacillus, Enterococcus, Alcaligenes, Klebsiella, Paenibacillus, Arthrobacter, Corynebacterium, Bacteriophage, Brevibacterium, Acanthoceras, Acanthococcus, Acarvochloris, Achnanthes, Achnanthidiun, Actinastrum, Actinochloris, Actinocyclus, Actinotaenium, Amphichrsis, Amphidiniunm, Amphikrikos, Amplhipleura, Amphiprora, Amphithrix, Amphora, Anabaena, Anabaenopsis, Aneumnastus, Ankistrodesmius, Ankyra, Anomoeoneis, Apatococcus, Aphanizomenon, Aphanocapsa, Aphanochaete, Aphanothece, Apiocystis, Apistonema, Arthrodesmus, Artherospira, Ascochloris, Asterionella, Asterococcus, Audouinella, Aularoseira, Bacillaria, Balbiania, Bambiusina, Bangia, Basichlamys, Batrarhospermum, Binurlearia, Bitrichia, Blidingia, Botrdiopsis, Botrydium, Botryococcus, Botryosphaerella, Brachiomonas, Brachysira, Brachytrichia, Brebissonia, Bulbochaete, Bumnilleria, Buinilleriopsis, Caloneis, Calothrix, Campylodiscus, Capsosiphon, Carteria, Catena, Cavinula, Cenritractus, Centroniella, Ceratiunt, Chaetoceros, Chaetochloris, Chaetomorpha, Chaetonella, Chaetonemna, Chaetopeltis, Chaetophora, Chaetosphaeridium, Chamaesiphion, Chara, Characiochloris, Characiopsis, Characium, Charales, Chilomonas, Chlainomonas, Chlamydoblepharis, Chlamydocapsa, Chlamydomonas, Chlamydomonopsis, Chlamydomnyxa, Chlamydonephris, Chlorangiella, Chlorangiopsis, Chlorella, Chlorobotrys, Chlorobrachis, Chlorochytrium, Chloroccun, Chlorogloea, Chlorogloeopsis, Chlorogonium, Chlorolobion, Chloromonas, Chlorophysema, Cholorphyta, Cholorosaccus, Cholorosarcina, Choricystis, Chromophyton, Chromulina, Chroococcidiopsis, Chrococcus, Chroodactylon, Chroomonas, Chroothece, Chrysamoeba, Chysapsis, Chrysidiastrum, Chrysocapsa, Chrysocapsella, Chrysochaete, Chrysohromulina, Chrysococcus, Chrysocrinus, Chrynsolepidomonas, Chrysolykos, Chrysonebula, Chrysophyta, Chrysopyxis, Chrysosaccus, Chrysophaerella, Chrysotephanosphaera, Clodophora, Clastidium, Closteriopsis, Closterium, Coccomyxa, Cocconeis, Coelastrella, Coelastrum, Coelosphaerium, Coenochloris, Coenococcus, Coenocystis, Colacium, Coleochaete, Collodictyon, Compsogonopsis, Compsopogon, Conjugatophyta, Conoehaete, Coronastrum, Cosmarium, Cosmnioneis, Cosmocladium, Crateriportula, Craticula, Crinalium, Crucigenia, Crucigeniella, Cryptoaulax, Cryptomonas, Cryptophyta, Ctenophora, Cyanodictyon, Cyanonephron, Cyanophora, Cyanophyta, Cyanothece, Cyanothomonas, Cyclonexis, Cyclostephanos, Cyclotella, Cylindrocapsa, Cylindrocystis, Cylindrospermum, Cylindrotheca, Cymatopleura, Cymbella, Cymbeilonitzschia, Cystodinium Dactylococcopsis, Debarya, Denticula, Dermatochrysis, Dermorarpa, Dermocarpella, Desmatractum, Desmidium, Desmococcus, Desmonema, Desmosiphon, Diacanthos, Diacronema, Diadesmis, Diatoma, Diatomella, Dicellula, Dichothrix, Dichtotomococcrus, Dicranochaete, Dictyochloris, Dictyococcus, Dictyosphaerium, Didymocystis, Didymogenes, Didymosphenia, Dilabifilum, Dimorphoccus, Dinobryon, Dinocuccus, Diplochloris, Diploneis, Diplostauron, Distrionella, Docidium, Draparnaldia, Dunaliella, Dysmorphaocuccus, Ecballocystis, Elakatothrix, Ellerbeckia, Encyonema, Enteromorpha, Entocladia, Entomoeis, Entophysalis, Ephichrysis, Epipyxis, Epithemia, Eremosphaura, Euastropsis, Euatstrum, Eucapsis, Eucocconeis, Eudorina, Euglena, Euglenophyta, Eunotia, Eustigmatophyta, Eutreptia, Fallcia, Ficherella, Fragilaria, Fragilariforma, Franceia, Frustulia, Curcilla, Geminella, Genicularia, Glaucocystis, Glaucophyta, Glenodiniopsis, Glenodinium, Gloeomonas, Gloeoplax, Gloeothece, Geloeotila, Gloeotrichia, Gloiodictyon, Golenkinia, Golenkiniopsis, Gomontia, Gomphocymbella, Gomphonema, Gomphosphaeria, Gonatozygon, Gongrosia, Gongrosira, Goniochloris, Gonium, Gonyostomum, Granulochloris, Granulocystopsis, Groenbladia, Gymnodiunium, Gymnozyga, Gyrosignma, Haematocuccus, Hafniomonas, Hallassia, Hammatoidea, Hannaea, Hantzchia, Hapalosiphon, Haplotaenium, Haptophyta, Haslea, Hemidinuim, Hemitonia, Heribaudiella, Heteromastix, Heterothrix, Hibberdia, Hildenbrandia, Hillea, Holopedium, Homoeothrix, Hormanthonema, Hormotila, Hyalobrachion, Hyalocardium, Hyalodiscus, Hyalogonium, Hyalotheca, Hydrianum, Hydrococcus, Hydrocoleum, Hydrocoryne, Hydrodictyon, Hydrosera, Hydrurus, Hyella, Hymenomonas, Isthmochloron, Johannesbaptistia, Juranyiella, Karayevia, Kathablepharis, Katodinium, Kaphyrion, Keratococcus, Kirchneriella, Klebsormidium, Kolbesia, Koliella, Komarekia, Korshikoviella, Kraskella, Lagerheimia, Lagynion, Lamprothamnium, Lemanea, Lepocinclis, Leptosira, Lobococcus, Lobocystis, Lobomonas, Luticola, Lynbya, Malleochloris, Mallomonas, Mantoniella, Marssoniella, Martyana, Mastigocloleus, Gastogloia, Melosira, Merismopedia, Mesostigma, Mesotaenium, Micractinium, Micrasterias, Microchaete, Microcoleus, Microcystis, Microglena, Micromonas, Microspora, Microthamnion, Mischococcus, Monocrysis, Monodus, Monomastix, Monoraphidium, Monostroma, Mougeotia, Mougeotiopsis, Myochloris, Myromecia, Myxocarcina, Naegeliella, Nannochloris, Nautoccus, Navicula, Neglectella, Neidium, Nephroclamys, Nephrocytium, Nephrodiella, Nephroselmis, Netrium, Nitella, Nitellopsis, Nitzschia, Nodularia, Nostoc, Ochromonas, Oedogonium, Oligochaetophora, Onychonema, Oocadrium, Oocrystis, Opephora, Ophiocytium, Orthoseira, Oscillartoria, Oxyneis, Pachycladella, Palmella, Palmodictyon, Pnadorina, Pannus, Paralia, Pascherina, Paulshulzia, Pediastrum, Pedinella, Pedinomonas, Pedinopera, Pelagodictyon, Penium, Peranema, Peridiniopsis, Peridinium, Peronia, Petroneis, Phacotus, Phacus, Phaeaster, Phaeodermatium, Phaeophyta, Phaeoshaera, Phaeothamnion, Phormidium, Phycopeltis, Phyllariochloris, Phyllocadium, Phyllomitas, Pinnilaria, Pitophora, Placoneis, Planctonema, Planktophaeria, Planothidium, Plectonema, Pleodorina, Pleurastrum, Pleurocapsa, Pleurocladia, Pleurodiscus, Pleurosigma, Pleurosira, Pleurotaenium, Pocillomanas, Podohedea, Polyblepharides, Polychaetophora, Polyedriella, Polyedriopsis, Polygoniochloris, Polyepidomanas, Polytaenia, Polytoma, Polytomella, Porphyridium, Posteriochromonas, Prasinochloris, Prasinocladus, Prasinophyta, Praisola, Prochlorphyta, Psammodictyon, Psammothidium, Pseudanabaena, Pseudenoclonium, Psuedocarteria, Pseudochate, Pseaudocharacium, Pseudococcomyxa, Pseudodictyosphaerium, Pseudokephyrion, Pseudoncobrysa, Pseudoquadrigula, Pseudophaerocystis, Pseudostaurastrum, Pseudostraurosira, Pyrrophyta, Quadrichloris, Quadricoccus, Quadrigula, Radiocuccus, Radiobetalum, Raphidiopsis, Raphidocelis, Raphidonema, Raphidophyta, Peimeria, Rhadorderma, Rhabomonas, Rhizoclonium, Rhodomonas, Rhodiphyta, Rhoicosenia, Rhopalodia, Rivularia, Rosenvingiella, Rossithidium, Roya, Scenedesmus, Scherffelia, Schizochlamydella, Schizochlamys, Schizomeris, Schizothrix, Schroederia, Scolioneis, Scotiella, Scotiellopsis, Scourfieldia, Scytonema, Slenastrum, Selenochloris, Sellaphora, Semiorbis, Siderocelis, Diderocystopsis, Dimonsenia, Siphononema, Sirocladium, Sirogonium, Skeletonema, Sorastrum, Spermatozopis, Sphaerellocystis, Sphaerellopsis, Sphaerodinium, Sphaeroplea, Sphaerozosma, Spiniferomonas, Spirogyra, Spirotaenia, Spirulina, Spondylomorum, Spondylosium, Sporotetras, Spumella, Staurastrum, Stauerodesmus, Stauroneis, Staurosira, Staurrosiella, Stenopterobia, Stephanocostis, Stephanodiscus, Stephanoporos, Stephanoshaera, Stichoccus, Stichogloea, Sigeoclonium, Stigonema, Stipitocuccus, Stokesiella, Stombomonas, Stylochrysalis, Stylodinium, Styloyxis, Stylosphaeridium, Surirella, Sykidion, Symploca, Synechococcus, Synechocystis, Synedra, Synochromonas, Synura, Tabellaria, Tabularia, Teilingia, Temnogametum, Tetmemorus, Tetrachlorella, Tetracyclus, Tetrademus, Tetraedriella, tetraedron, Tetraselmis, Tetraspora, Tetrastrum, Thalassiosira, Thanmiochaete, Thoakochloris, Thorea, Tolypella, Tolypothrix, Trachelomonas, Trachydiscus, Trebouxia, Trentepholia, Treubaria, Tribonema, Trichodesmium, Tricodiscus, Trochiscia, Tryblionella, Ulothrix, Uroglena, Uronema, Urosolenia, Urospora, Uva, Vacuolaria, Vaucheria, Volvox, Volvulina, Westella, Woloszynskia, Xanthidium, Xanthophyta, Exencoccus, Zygenema, Zygnemopsis, and Zygonium.

Specific examples of bacteria cells include Escherichia coli; Oligotropha carboxidovorans, Pseudomononas sp. Alcaligenes eutrophus (Cupriavidus necator), Bacillus licheniformis, Paenibacillus macerans, Rhodococcus erythropolis, Pseudomonas putida, Lactobacillus plantarum, Enterococcus faecium, Enterococcus gallinarium, Enterococcus faecalis, Bacillus subtilis, Cupriavidus basilensis, Cupriavidus campinensis, Cupriavidus gilardi, Cupriavidus laharsis, Cupriavidus metallidurans, Cupriavidus oxalaticus, Cupriavidus pauculus, Cupriavidus pinatubonensis, Cupriavidus respiraculi, Cupriavidus taiwanensis, In some aspects, the bacterium is Nocardia sp. NRRL 5646, Nocardia farcinica, Streptomyces griseus, Salinispora arenicola, or Clavibacter michiganenesis.

The recombinant cell may be a synthetic cell or a cell produced by a synthetic genome, as described in U.S. Patent Publication 2007/0264688, or 2007/0269862. The cell may be a CHO cell, a COS cell, a VERO cell, a BHK cell, a HeLa cell, a Cv1 cell, an MDCK cell, a 293 cell, a 3T3 cell, or a PC12 cell.

In some aspects, this application discloses a recombinant cell with an engineered biosynthetic pathway comprising one or more heterologous enzymes that convert chemical precursors and/or substrates into desired chemical products. In some cases, the engineered biosynthetic pathway comprises heterologous enzymes to synthesize the desired chemical product. In some instances, individual heterologous enzymes work in a stepwise fashion to convert a precursor into the desired chemical product. The engineered biosynthetic pathway can comprise one or more heterologous enzymes having 3-ketoacyl-CoA synthase activity to produce fatty acid chain products of C6-C10 chain length. The engineered biosynthetic pathway can also comprise genetically modifications for reduction in activity of one or more thioesterases such as YjjU, YbaW, FrmB, TesA and/or YdiI. Also, the engineered biosynthetic pathway can also comprise genetic modifications to reduce the activity of other enzymes such as BioH. Additionally, the engineered biosynthetic pathway can also comprise genetic modifications to increase the activity of other enzymes such as ACS.

In some aspects, the recombinant cell comprises one or more 3-ketoacyl-CoA synthases to catalyze the reaction of acyl-CoA with malonyl-CoA to produce fatty acids and fatty acid chain products of C6-C10 chain length. However, the reaction of acyl-CoA with malonyl-CoA produces a 3-ketoacyl-CoA compound that must be reduced to the corresponding acyl compound before it can condense with another molecule of malonyl-CoA to extend the chain. The reduction takes place in three steps, the first being the reduction of the 3-ketoacyl group to the corresponding 3-hydroxyacyl group. The second reaction is a dehydration to the corresponding trans-2-enoylacyl compound, which is reduced in a third step to the corresponding acyl-CoA. The first reaction step is enzymatically catalyzed by a keto-CoA reductase (KCR) enzyme (E.C. 1.1.1.35). The second step is enzymatically catalyzed by a 3-hydroxy-acyl-CoA dehydratase (3HDh) enzyme (E.C. 4.2.1.17). Some bifunctional enzymes catalyze both of the first and second step reactions (E.C. 1.1.1.35 and E.C. 4.2.1.55). The third reaction step is enzymatically catalyzed by an enoyl-CoA reductase (ECR) enzyme (E.C. 1.1.1.32).

Accordingly, the engineered biosynthetic pathway preferably further comprises at least one of (1) a heterologous KCR gene that encodes for a KCR enzyme; (2) a heterologous 3HDh gene that encodes for a 3HDh enzyme; (3) a heterologous gene that encodes for a bifunctional enzyme that catalyzes both of the first and second reaction steps (E.C. 1.1.1.35 and 4.1.2.55) and (4) a heterologous ECR gene that encodes for an ECR enzyme. Preferably, the recombinant cell contains at least (1), (2) and (4) or at least (3) and (4). In each case, the gene preferably is under the control of promoter and/or terminator sequences active in the recombinant cell.

The KCR enzyme may be, for example, one encoded by a P. aeruginosa fadB gene and/or having an amino acid sequence at least 80%, at least 90%, at least 95%, at least 99% or 100% identical to SEQ ID NO: 8, one encoded by a P. aeruginosa fadG gene and/or having an amino acid sequence at least 80%, at least 90%, at least 95%, at least 99% or 100% identical to SEQ ID NO: 9, one encoded by a C. beijerinckii hbd gene and/or having an amino acid sequence at least 80%, at least 90%, at least 95%, at least 99% or 100% identical to SEQ ID NO: 10, and others as described in WO 2015/010103.

The 3HDh enzyme may be, for example, one encoded by a C. acetobutylicum crt (short-chain-enoyl-CoA hydratase) gene and/or having an amino acid sequence at least 80%, at least 90%, at least 95%, at least 99% or 100% identical to SEQ ID NO: 11, one encoded by a P. putida ech (enoyl-CoA hydratase/aldolase) gene and/or having an amino acid sequence at least 80%, at least 90%, at least 95%, at least 99% or 100% identical to SEQ ID NO: 12, and others as described in WO 2015/010103.

Suitable bifunctional enzymes that catalyze both the first and second reactions steps include, for example, one encoded by an E. coli fadB gene and/or having an amino acid sequence at least 80%, at least 90%, at least 95%, at least 99% or 100% identical to SEQ ID NO: 8; one encoded by an R. novegicus ech2 gene, and others as described in WO 2015/010103.

Suitable ECR enzymes include, for example, one encoded by a T. denticola ter gene and/or having an amino acid sequence at least 80%, at least 90%, at least 95%, at least 99% or 100% identical to SEQ ID NO: 13.

The recombinant cell can further include at least one heterologous 3-ketobutyryl-CoA synthase gene, different from the modified 3-ketoacyl-CoA synthases described above, which encodes for a 3-ketobutyryl-CoA synthase. The heterologous 3-ketobutyryl-CoA synthase gene may encode for a 3-ketobutyryl-CoA synthase enzyme that is at least 80%, at least 90%, at least 95%, at least 99% or 100% identical to any of those identified as SEQ ID NO: 1-120 of WO 2015/10103.

In some aspects, the heterologous 3-ketobutyryl-CoA synthase gene is a Streptomyces Sp CL190 gene and/or a gene that encodes for an NphT7 enzyme that is at least 80%, at least 90%, at least 95%, at least 99% or 100% identical to SEQ ID NO: 14.

In some aspects, the recombinant cell includes at least one gene that encodes for a modified NphT7 enzyme as described in WO 2015/10103. The modified NphT7 enzyme comprises an amino acid sequence having at least 70% but less than 100% to SEQ ID NO: 14. The modified NphT7 enzyme may have, for example, one or more amino acid substitutions selected from the group consisting of H100L, I147T, F217V, Y144L, V157F, G309S, G288S, a PDRP to HFLQ substitution for amino acid residues 86-89, I147F, I147M, I147Q, I147S, I147C, I147E, I147N, I147W, I147D, I147R, I147P, I147L, V196G, I147G, I147H, I147K, I147V, I147A, I147Y, F217G, F217A, F217L, F217I, F217M, F217T, F217P, F217S, F217E, F217L, F217V, F217W, S323A and S323V, and any combination of any two or more thereof.

In some aspects, the modified NphT7 enzyme comprises at least one amino acid substitution selected from the group consisting of I147V, I147S, I147T, and at least one additional amino acid substitution selected from H100L, F217V, S323A and S323V. In some aspects, the modified NphT7 enzyme corresponds to SEQ ID NO: 15. In some aspects, the modified NphT7 enzyme comprises an I147V, I147S or I147T amino acid substitution and an S323A amino acid substitution (corresponding to SEQ ID NO: 15 in which amino acid 100 is H, amino acid 147 is V, S or T, amino acid 217 is F and amino acid 323 is A). In some aspects, the modified NphT7 enzyme comprises an H100L substitution, an I147V, I147S or I147T amino acid substitution, an F217V substitution and an S323A amino acid substitution (corresponding to SEQ ID NO: 16 in which amino acid residue 100 is L, amino acid residue 147 is V, S or T, amino acid residue 217 is V and amino acid residue 323 is A).

In some aspects, the recombinant cell includes both of (1) a Streptomyces Sp CL190 nphT7 gene and/or a gene that encodes for an NphT7 enzyme that is at least 80%, at least 90%, at least 95%, at least 99% or 100% identical to SEQ ID NO: 14 and (2) a modified NphT7 enzyme having one or more amino acid substitutions selected from the group consisting of H100L, I147T, F217V, Y144L, V157F, G309S, G288S, a PDRP to HFLQ substitution for amino acid residues 86-89, I147F, I147M, I147Q, I147S, I147C, I147E, I147N, I147W, I147D, I147R, I147P, I147L, V196G, I147G, I147H, I147K, I147V, I147A, I147Y, F217G, F217A, F217L, F217I, F217M, F217T, F217P, F217S, F217E, F217L, F217V, F217W, S323A and S323V, and any combination of any two or more thereof. In some aspects, the recombinant cell includes a gene that encodes for an enzyme having SEQ ID NO: 14 and another gene that encodes for an enzyme having SEQ ID NO: 15. In other aspects, the recombinant cell includes a gene that encodes for an enzyme having SEQ ID NO: 14 and another gene that encodes for an enzyme having SEQ ID NO: 17 in which amino acid residue 100 is H or L, amino acid residue 147 is S, T or V, amino acid residue 271 is F or V and amino acid residue 323 is A.

The recombinant cell can further include at least one heterologous 3-ketoacyl-CoA synthase gene, different from the 3-ketoacyl-CoA synthases described above, which encodes for a 3-ketoacyl-CoA synthase. The heterologous 3-ketoacyl-CoA synthase gene may encode for a 3-ketoacyl-CoA synthase enzyme that is at least 80%, at least 90%, at least 95%, at least 99% or 100% identical to any of those identified as SEQ ID NO: 1-81, 86-93, 97, 105-116, and 119-171 in PCT/US18/16394.

In some aspects, the 3-ketoacyl-CoA synthase gene is an Acinetobacter schindleri CIP 107287 gene (Designation: Asch) and/or a gene that encodes for a 3-ketoacyl-CoA synthase enzyme that is at least 80%, at least 90%, at least 95%, at least 99% or 100% identical to SEQ ID NO: 18.

In some aspects, the recombinant cell includes at least one gene that encodes for a modified Asch enzyme as described in PCT/US18/16394. The modified Asch enzyme comprises an amino acid sequence having at least 70% but less than 100% sequence identity to SEQ ID NO: 26. The modified Asch enzyme may have, for example, one or more amino acid substitutions selected from the group consisting of T184I, F236L, V268A, V296A, V317A, and S328G, and any combination of any two or more thereof. The modified Asch enzyme may have altered specific activities relative to an unmodified Asch enzyme. For example, a modified Asch enzyme may have amino acid substitutions comprising T184I, F236L, V268A, V296A, V317A, and S328G (SEQ ID NO: 19) and may result in C8 FAME as the most abundant product. A modified Asch enzyme may have amino acid substitutions comprising T184I, V296A, and V268A (SEQ ID NO: 20) and may result in a mixture of C8 FAME and C10 FAME as the most abundant products. A modified Asch enzyme may have amino acid substitutions comprising V296A (SEQ ID NO: 21) and may result in C10 FAME as the most abundant product.

The recombinant cell can produce one or more enzymes that terminate the acyl elongation cycle and produce a product having the desired chain length. Such a termination enzyme may or may not be heterologous. The selection of termination enzyme may depend on whether the desired product is a fatty acid or a derivative thereof such as a fatty alcohol, a fatty aldehyde, a fatty alkene, a fatty amide, a fatty ester or a fatty alkane.

In some aspects the recombinant cell includes a gene that encodes for an ester synthase, in which case the product typically is a fatty acid ester. Suitable ester synthases have amino acid sequences at least 80%, at least 90%, at least 95%, at least 99% or at least 100% identical to any of the Marinobacter aquacolei Maq1 enzyme (SEQ ID NO: 289 of WO 2015/10103), the Psychrobacter cryohaloentis Pcry1 enzyme (SEQ ID NO: 290 of WO 2015/10103), the Rhodococcus jostii Rjos1 enzyme (SEQ ID NO: 291 of WO 2015/10103), the, Alcanivorax borkumensis strain SK2 Abork1 enzyme (SEQ ID NO: 292 of WO 2015/10103) and the Hahella chejuensis hche gene (SEQ ID NO: 22). The ester synthase may have an amino acid sequence at least 80%, at least 90%, at least 95%, at least 99% or at least 100% identical to the Hahella chejuensis Hche ester synthase (SEQ ID NO: 20).

The recombinant cell may also include one or more genes that encode for one or more of a fatty acyl-CoA reductase (alcohol or aldehyde forming), a fatty aldehyde reductase, an acyl-ACP reductase, an acyl-CoA:ACP acyltransferase, an acyl-CoA hydrolase, a carboxylic acid reductase, an aldehyde dehydrogenase and/or an acyl-ACP reductase.

The recombinant cell also may include (A) one or more genes that encode for a carboxyl transferase subunit α enzyme, (EC 6.3.1.2) such as an E. coli accA enzyme or an enzyme that is at least 80%, at least 90%, at least 95% or at least 99% identical thereto; (B) one or more genes that encode for a biotin carboxyl carrier protein, (EC 6.4.1.2) such as an E. coli accB enzyme or an enzyme that is at least 80%, at least 90%, at least 95% or at least 99% identical thereto; (C) one or more genes that encode for a biotin carboxylase subunit enzyme, (EC 6.3.4.14) such as an E. coli accC enzyme or an enzyme that is at least 80%, at least 90%, at least 95% or at least 99% identical thereto; (D) a carboxyl transferase subunit β (EC 6.4.1.2), such as an E. coli accD enzyme or an enzyme that is at least 80%, at least 90%, at least 95% or at least 99% identical thereto, (E) a fused E. coli accD subunit and accA subunit enzyme (SEQ ID NO: 23); or a combination of any two or more thereof. In some aspects, all of (A)-(E) are present.

In some aspects, the recombinant cell described here further comprises one or more additional genetic modifications to reduce or eliminate the expression of certain endogenous enzymes in the recombinant cell. Reducing or eliminating the expression of these certain endogenous enzymes in the recombinant cell can increase the production of desired products such as fatty acids and/or fatty acid chain products. These reduced or eliminated endogenous enzymes include one or more of the following enzymes:

Methylglyoxal synthase (EC 4.2.3.3), for example that encoded by the E. coli mgsA gene.
Lactate dehydrogenase (EC 1.1.1.27), for example that encoded by the E. coli ldhA gene.
Phosphotransacetylase (EC 2.3.1.8), for example that encoded by the E. coli pta gene.
Acetate kinase (EC 2.7.2.1), for example that encoded by E. coli ackA gene.
Acyl-CoA synthetase (EC 6.2.1.3), for example that encoded by the E. coli fadD gene.
Pyruvate formate lyase (EC 2.3.1.54), for example that encoded by the E. coli pflB gene.
Pyruvate oxidase (EC 1.2.2.2), for example that encoded by the E. coli poxB gene.
Fused acetaldehyde-CoA dehydrogense (EC 1.2.1.10), for example that encoded by the E. coli adhE gene.
Trigger factor (EC 5.2.1.8), for example that encoded by the E. coli tig gene.
Restriction endonuclease (EC 3.1.21.3), for example that encoded by the E. coli hsdr514 gene.
The atoDAEB operon.
Acyl-CoA thioesterase (EC 3.1.2.-), for example that encoded by the E. coli tesB or yciA gene.
Acyl-coenzyme A dehydrogenase (EC 1.3.8.7), for example that encoded by the E. coli fadE gene.
3-ketoacyl-CoA thiolase (EC 2.3.1.16), for example that encoded by the E. coli fadA gene.
L-ribulokinase (EC2.7.1.16), for example that encoded by the E. coli araB gene.
L-ribulose-5-phosphate-4-epimerase (EC 5.1.3.4), for example that encoded by the E. coli araD gene.
Beta-D-galactosidase (EC 3.2.1.23), for example that encoded by the E. coli lacZ gene.
Lambda phage lysogen.
Rhamnulose-1-phosphate aldolase (EC 4.1.2.19), for example that encoded by the E. coli rhaD gene.
Rhamnulokinase (EC 2.7.1.5), for example that encoded by the E. coli rhaB gene.
F mating factor.
Truncated RNase PH (EC2.7.7.56) Rph-1 gene.

Other genetic modifications may be present in the recombinant cell, including any of those described in WO 2015/10103.

Any heterologous gene may be operatively linked to a promoter and/or terminator sequence that is functional in the recombinant strain. The promoter may be an inducible promoter that functions only under certain conditions. For example, a low phosphate inducible promoter such as the promoter of the wild-type E. coli phoE gene (PphoE), is a useful promoter for the 3-ketoacyl synthase gene. Such a promoter is active in a low phosphate environment. Accordingly, a recombinant cell in which the 3-ketoacyl synthase gene is under the control of an E. coli phoE promoter or another low phosphate inducible promoter may be cultivated in a fermentation medium containing no more than 25 mM phosphate, especially no more than 20 mM, no more than 2 mM, no more than 1 mM, no more than 0.5 mM, or no more than 0.25 mM phosphate. In some aspects, the promoter that is a low phosphate inducible promoter is the promoter for the pstS gene (PpstS). This promoter may be constructed to include a binding site for Integration Host Factor and is thus designated PpstSIH (Lyzen et al., Plasmid 60:125 (2008)).

Any heterologous gene may be integrated into the genome of the recombinant strain and/or present in one or more plasmids. If integrated into the genome, the heterologous gene may be inserted at a targeted or random location. Transformation methods such as electroporation and chemical methods (including calcium chloride and/or lithium acetate methods) known in the art are suitable. Examples of suitable transformation methods are described, for example, in Molecular Cloning: A Laboratory Manual, 4^{th} Ed. Spring Harbor Press 2012. In general, no special transformation methods are necessary to produce the recombinant cells.

Deletions and/or disruptions of native genes can be performed by transformation methods, by mutagenesis and/or by forced evolution methods. In mutagenesis methods, cells are exposed to ultraviolet radiation or a mutagenic substance, under conditions sufficient to achieve a high kill rate (60-99.9%, preferably 90-99.9%) of the cells. Surviving cells are then plated and selected or screened for cells having the deleted or disrupted metabolic activity. Disruption or deletion of the desired native gene(s) can be confirmed through PCR or Southern analysis methods.

The recombinant cells described herein are used to produce compounds having a straight-chain alkyl group. The recombinant cells are grown under conditions such that they produce such compounds, and the compounds are recovered.

When the recombinant cell is a plant cell, the plant can be grown and the compound having the straight-chain alkyl group can be recovered from the plant or any portion thereof, such as roots, stems, leaves, flowers, seeds, seed pods and the like, in which the compound accumulates during the growth of the plant.

Single-cell and other microcells can be used in a culturing process to produce such compounds.

Culturing is performed generally by forming a culture medium that includes at least one carbon source that is capable of being metabolized by the recombinant cell to produce the product compounds and nutrients as may be required by the specific recombinant cell. The nutrients may include, for example, at least one nitrogen source such as yeast extract, peptone, tryptone, soy flour, corn steep liquor, or casein, at least one phosphorus source, one or more vitamins such as biotin, vitamin B12 and derivatives of vitamin B12, thiamin, pantothenate, one or more trace metals and the like. The fermentation medium may also contain additional materials such as anti-foam agents, biocides, buffers and the like.

In some cases, such as the production of fatty acid esters, the culture medium may also include a reagent that reacts with the straight-chain compound to produce the desired product. In the specific case of fatty acid esters, for example the culture medium preferably contains an alkanol such as methanol, ethanol or a C3-C8 alkanol. The alkanol reacts to produce the corresponding ester. A native or heterologous ester synthase, or other appropriate enzyme, may be expressed by the recombinant cell to catalyze such a reaction.

Generally, the culture medium is inoculated with the recombinant cell, and the inoculum is cultured in the medium so that the cell density increases to a cell density suitable for production. The culture medium is then maintained at conditions sufficient for the recombinant cells to produce the desired product.

Suitable culture conditions will of course depend on the requirements of the particular recombinant strain. The temperature of the culture medium may be, for example from 20° C. to 70° C., with a temperature of 25 to 40° C. being preferred for most recombinant cells.

The pH of the culture medium may be, for example, from 2.0 to 10.0, from 3.0 to 9.0 or from 6.0 to pH 8.5.

It is contemplated that the described aspects may be practiced using either batch, fed-batch or continuous processes and that any known mode of bio-production would be suitable.

The culturing may be performed under aerobic, microaerobic, or anaerobic conditions, as required or can be tolerated by the particular recombinant cell. Generally, no special culturing equipment is needed to perform the fermentation. The equipment may include, for example, a tank suitable for holding the recombinant cell and the culture medium; a line for discharging contents from the culture tank to an extraction and/or separation vessel; and an extraction and/or separation vessel suitable for removal of the chemical product from cell culture waste.

The carbon source is one or more carbon-containing compounds that can be metabolized by the recombinant cell as a source of carbon. Examples of suitable carbon sources include sugars such as glucose, sucrose, fructose, lactose, C-5 sugars such as xylose and arabinose, glycerol and polysaccharides such as starch and cellulose. Other suitable carbon sources include fermentable sugars as may be obtained from cellulosic and lignocellulosic biomass through processes of pretreatment and saccharification, as described, for example, in U.S. Patent Publication No. 2007/0031918A1, hemicellulose, lignin, starch, oligosaccharides and/or monosaccharides. Other suitable carbon sources include high-fructose corn syrup, cheese whey permeate, cornsteep liquor, sugar beet molasses, and barley malt. Still other suitable carbon sources include carbon dioxide, carbon monoxide, methanol, methylamine and glucosamine.

The culturing process may be continued until a titer of the desired product reaches at least 0.01, at least 0.05, at least 0.1, at least 0.25, at least 0.5 or at least 1 g per liter of culture medium (g/L). The fermentation process may be continued until the titer reaches, for example, up to 40, up to 45, up to 50, up to 80, up to 100, or up to 120 g/L. The specific productivity may be, for example, from 0.01 and 0.60 grams of the desired product per gram of cells on a dry weight basis per hour (g chemical product/g DCW-hr). The volumetric productivity achieved may be at least 0.005 g of the desired product per liter per hour (g/L-hr), at least 0.01 g/L-hr, at least 0.1 g/L-hr or at least 0.5 g/L-hr, and may be up to, for example, 10 g/L-hr, up to 5 g/L-hr or up to 1 g/L-hr. [0212] In some aspects, specific productivity as measured over a 24-hour fermentation (culture) period may be greater than about 0.01, 0.05, 0.10, 0.20, 0.5, 1.0, 2.0, 3.0, 4.0, 5.0, 6.0, 7.0, 8.0, 9.0, 10.0, 11.0 or 12.0 grams of chemical product per gram DCW of cells (based on the final DCW at the end of the 24-hour period).

In some aspects, recombinant cells are disclosed that are genetically engineered to produce fatty acid esters. In other aspects, these recombinant cells that are genetically engineered to produce fatty esters are further genetically engineered for other desired traits as compared to recombinant cells that have not been further genetically engineered. For example, these recombinant cells that are genetically engineered to produce fatty acid esters can be further genetically engineered to produce a reduction in total free fatty acids, to produce a reduction in C8 and/or C10 free fatty acids, to produce a decreased ratio of total free fatty acids to fatty acid esters, to produce an increase in fatty acid esters, and/or to produce an increase in C8 and/or C10 fatty acid esters, all compared to recombinant cells that have not been further genetically engineered. In some aspects, one or more of these desired traits can be determined by culturing recombinant cells that are genetically engineered to produce fatty esters and recombinant cells that are genetically engineered to produce fatty esters that are further genetically engineered for other desired traits and comparing results, for example the results of production of fatty acid ester and free fatty acid. The culturing of recombinant cells that are genetically engineered to produce fatty acid esters and recombinant cells that are genetically engineered to produce fatty acid esters that are further genetically engineered for other desired traits is carried out with identical culturing protocols to compare the results, for example a shake flask protocol as described below in Example 1 can be followed and the results of each culture can be compared. In some aspects, the shake flask protocol as described below in Example 1 can be used to determine effect(s) of deletion of one or more genes and/or overexpression of one or more genes on desired traits, such as for example reduction in total free fatty acids, reduction in C8 and/or C10 free fatty acids, decreased ratio of total free fatty acids to fatty acid esters, increase in total fatty acid esters, and/or increase in C8 and/or C10 fatty acid esters.

In some aspects, a recombinant cell produces fatty acid ester and is genetically engineered for reduced activity of a polypeptide encoded by a gene with an amino acid sequence set forth in SEQ ID NO: 1 and corresponds to the polypeptide designated as YjjU. In other aspects, the polypeptide encoded by a gene with an amino acid sequence designated as YjjU comprises a polypeptide having more than 50%, more than 55%, more than 60%, more than 65%, more than 70%, more than 80%, more than 85%, more than 90%, more than 91%, more than 92%, more than 93%, more than 94%, more than 95%, more than 96%, more than 97%, more than 98%, or more than 99% sequence identity to the amino acid sequence of SEQ ID NO: 1.

In some aspects, the recombinant cell producing fatty acid ester and genetically engineered for reduced activity of the polypeptide corresponding to YjjU, produces a reduction in total free fatty acids compared to a similar recombinant cell that has not been genetically engineered for reduced activity of the polypeptide corresponding to YjjU. In other aspects, the recombinant cell producing fatty acid ester and genetically engineered for reduced activity of the polypeptide corresponding to YjjU, produces a reduction in total free fatty acids of 1%, 2%, 3%, 4%, 5%, 10%, 15%, 20%, 25%, 30%, 40%, 50%, 60%, 70%, 80%, 90% or more compared to a similar recombinant cell that has not been genetically engineered for reduced activity of the polypeptide corresponding to YjjU.

In some aspects, the recombinant cell producing fatty acid ester and genetically engineered for reduced activity of the polypeptide corresponding to YjjU, produces a reduction in C10 free fatty acids compared to a similar recombinant cell that has not been genetically engineered for reduced activity of the polypeptide corresponding to YjjU. In other aspects, the recombinant cell producing fatty acid ester and genetically engineered for reduced activity of the polypeptide corresponding to YjjU, produces a reduction in C10 free fatty acids of 1%, 2%, 3%, 4%, 5%, 10%, 15%, 20%, 25%, 30%, 40%, 50%, 60%, 70%, 80%, 90% or more compared to a similar recombinant cell that has not been genetically engineered for reduced activity of the polypeptide corresponding to YjjU.

In some aspects, the recombinant cell producing fatty acid ester and genetically engineered for reduced activity of the polypeptide corresponding to YjjU and combined with a Asch(V296A) variant, produces a reduction in C10 free fatty acids compared to a similar recombinant cell that has not been genetically engineered for reduced activity of the polypeptide corresponding to YjjU. In other aspects, the recombinant cell producing fatty acid ester and genetically engineered for reduced activity of the polypeptide corresponding to YjjU, produces a reduction in C10 free fatty acids of 1%, 2%, 3%, 4%, 5%, 10%, 15%, 20%, 25%, 30%, 40%, 50%, 60%, 70%, 80%, 90% or more compared to a similar recombinant cell that has not been genetically engineered for reduced activity of the polypeptide corresponding to YjjU.

In some aspects, the recombinant cell producing fatty acid ester and genetically engineered for reduced activity of the polypeptide corresponding to YjjU, produces a decreased ratio of total free fatty acids to fatty acid esters compared to a similar recombinant cell that has not been genetically engineered for reduced activity of the polypeptide corresponding to YjjU. In other aspects, the recombinant cell producing fatty acid ester and genetically engineered for reduced activity of the polypeptide corresponding to YjjU, produces a decreased ratio of total free fatty acids to fatty acid esters of 1%, 2%, 3%, 4%, 5%, 10%, 15%, 20%, 25%, 30%, 40%, 50%, 60%, 70%, 80%, 90% or more compared to a similar recombinant cell that has not been genetically engineered for reduced activity of the polypeptide corresponding to YjjU.

In some aspects, the recombinant cell producing fatty acid ester and genetically engineered for reduced activity of the polypeptide corresponding to YjjU, produces an increase in total fatty acid esters compared to a similar recombinant cell that has not been genetically engineered for reduced activity of the polypeptide corresponding to YjjU. In other aspects, the recombinant cell producing fatty acid ester and genetically engineered for reduced activity of the polypeptide corresponding to YjjU, produces an increase in fatty acid esters of 1%, 2%, 3%, 4%, 5%, 10%, 15%, 20%, 25%, 30%, 40%, 50%, 60%, 70%, 80%, 90% or more compared to a similar recombinant cell that has not been genetically engineered for reduced activity of the polypeptide corresponding to YjjU.

In some aspects, the recombinant cell producing fatty acid ester and genetically engineered for reduced activity of the polypeptide corresponding to YjjU and combined with a Asch(V296A) variant, produces an increase in C10 fatty acid esters compared to a similar recombinant cell that has not been genetically engineered for reduced activity of the polypeptide corresponding to YjjU. In other aspects, the recombinant cell producing fatty acid ester and genetically engineered for reduced activity of the polypeptide corresponding to YjjU, produces an increase in C10 fatty acid esters of 1%, 2%, 3%, 4%, 5%, 10%, 15%, 20%, 25%, 30%, 40%, 50%, 60%, 70%, 80%, 90% or more compared to a similar recombinant cell that has not been genetically engineered for reduced activity of the polypeptide corresponding to YjjU.

In some aspects, a method for decreasing free fatty acid production comprises culturing a recombinant cell that produces fatty acid ester and is genetically engineered for reduced activity of a polypeptide encoded by a gene with an amino acid sequence set forth in SEQ ID NO: 1 and corresponds to the polypeptide designated as YjjU. In other aspects, the polypeptide encoded by a gene with an amino acid sequence designated as YjjU comprises a polypeptide having more than 50%, more than 55%, more than 60%, more than 65%, more than 70%, more than 80%, more than 85%, more than 90%, more than 91%, more than 92%, more than 93%, more than 94%, more than 95%, more than 96%, more than 97%, more than 98%, or more than 99% sequence identity to the amino acid sequence of SEQ ID NO: 1.

In some aspects, method for decreasing free fatty acid production comprises culturing the recombinant cell producing fatty acid ester and genetically engineered for reduced activity of the polypeptide corresponding to YjjU to produce a reduction in total free fatty acids compared to a similar recombinant cell that has not been genetically engineered for reduced activity of the polypeptide corresponding to YjjU. In other aspects, the recombinant cell producing fatty acid ester and genetically engineered for reduced activity of the polypeptide corresponding to YjjU, produces a reduction in total free fatty acids of 1%, 2%, 3%, 4%, 5%, 10%, 15%, 20%, 25%, 30%, 40%, 50%, 60%, 70%, 80%, 90% or more compared to a similar recombinant cell that has not been genetically engineered for reduced activity of the polypeptide corresponding to YjjU.

In some aspects, method for decreasing free fatty acid production comprises culturing the recombinant cell producing fatty acid ester and genetically engineered for reduced activity of the polypeptide corresponding to YjjU to produce a reduction in C10 free fatty acids compared to a similar recombinant cell that has not been genetically engineered for reduced activity of the polypeptide corresponding to YjjU. In other aspects, the recombinant cell producing fatty acid ester and genetically engineered for reduced activity of the polypeptide corresponding to YjjU, produces a reduction in C10 free fatty acids of 1%, 2%, 3%, 4%, 5%, 10%, 15%, 20%, 25%, 30%, 40%, 50%, 60%, 70%, 80%, 90% or more compared to a similar recombinant cell that has not been genetically engineered for reduced activity of the polypeptide corresponding to YjjU.

In some aspects, method for decreasing free fatty acid production comprises culturing the recombinant cell producing fatty acid ester and genetically engineered for reduced activity of the polypeptide corresponding to YjjU and combined with a Asch(V296A) variant to produce a reduction in C10 free fatty acids compared to a similar recombinant cell that has not been genetically engineered for reduced activity of the polypeptide corresponding to YjjU. In other aspects, the recombinant cell producing fatty acid ester and genetically engineered for reduced activity of the polypeptide corresponding to YjjU, produces a reduction in C10 free fatty acids of 1%, 2%, 3%, 4%, 5%, 10%, 15%, 20%, 25%, 30%, 40%, 50%, 60%, 70%, 80%, 90% or more compared to a similar recombinant cell that has not been genetically engineered for reduced activity of the polypeptide corresponding to YjjU.

In some aspects, a recombinant cell produces fatty acid ester and is genetically engineered for reduced activity of a polypeptide encoded by a gene with an amino acid sequence set forth in SEQ ID NO: 2 and corresponds to the polypeptide designated as YbaW. In other aspects, the polypeptide encoded by a gene with an amino acid sequence designated as YbaW comprises a polypeptide having more than 50%, more than 55%, more than 60%, more than 65%, more than 70%, more than 80%, more than 85%, more than 90%, more than 91%, more than 92%, more than 93%, more than 94%, more than 95%, more than 96%, more than 97%, more than 98%, or more than 99% sequence identity to the amino acid sequence of SEQ ID NO: 2.

In some aspects, the recombinant cell producing fatty acid ester and genetically engineered for reduced activity of the polypeptide corresponding to YbaW, produces a reduction in total free fatty acids compared to a similar recombinant cell that has not been genetically engineered for reduced activity of the polypeptide corresponding to YbaW. In other aspects, the recombinant cell producing fatty acid ester and genetically engineered for reduced activity of the polypeptide corresponding to YbaW, produces a reduction in total free fatty acids of 1%, 2%, 3%, 4%, 5%, 10%, 15%, 20%, 25%, 30%, 40%, 50%, 60%, 70%, 80%, 90% or more compared to a similar recombinant cell that has not been genetically engineered for reduced activity of the polypeptide corresponding to YbaW.

In some aspects, the recombinant cell producing fatty acid ester and genetically engineered for reduced activity of the polypeptide corresponding to YbaW, produces a reduction in C10 free fatty acids compared to a similar recombinant cell that has not been genetically engineered for reduced activity of the polypeptide corresponding to YbaW. In other aspects, the recombinant cell producing fatty acid ester and genetically engineered for reduced activity of the polypeptide corresponding to YbaW, produces a reduction in C10 free fatty acids of 1%, 2%, 3%, 4%, 5%, 10%, 15%, 20%, 25%, 30%, 40%, 50%, 60%, 70%, 80%, 90% or more compared to a similar recombinant cell that has not been genetically engineered for reduced activity of the polypeptide corresponding to YbaW.

In some aspects, the recombinant cell producing fatty acid ester and genetically engineered for reduced activity of the polypeptide corresponding to YbaW and combined with a Asch(V296A) variant, produces a reduction in C10 free fatty acids compared to a similar recombinant cell that has not been genetically engineered for reduced activity of the polypeptide corresponding to YbaW. In other aspects, the recombinant cell producing fatty acid ester and genetically engineered for reduced activity of the polypeptide corresponding to YbaW, produces a reduction in C10 free fatty acids of 1%, 2%, 3%, 4%, 5%, 10%, 15%, 20%, 25%, 30%, 40%, 50%, 60%, 70%, 80%, 90% or more compared to a similar recombinant cell that has not been genetically engineered for reduced activity of the polypeptide corresponding to YbaW.

In some aspects, the recombinant cell producing fatty acid ester and genetically engineered for reduced activity of the polypeptide corresponding to YbaW, produces a decreased ratio of total free fatty acids to fatty acid esters compared to a similar recombinant cell that has not been genetically engineered for reduced activity of the polypeptide corresponding to YbaW. In other aspects, the recombinant cell producing fatty acid ester and genetically engineered for reduced activity of the polypeptide corresponding to YbaW, produces a decreased ratio of total free fatty acids to fatty acid esters of 1%, 2%, 3%, 4%, 5%, 10%, 15%, 20%, 25%, 30%, 40%, 50%, 60%, 70%, 80%, 90% or more compared to a similar recombinant cell that has not been genetically engineered for reduced activity of the polypeptide corresponding to YbaW.

In some aspects, the recombinant cell producing fatty acid ester and genetically engineered for reduced activity of the polypeptide corresponding to YbaW, produces an increase in total fatty acid esters compared to a similar recombinant cell that has not been genetically engineered for reduced activity of the polypeptide corresponding to YbaW. In other aspects, the recombinant cell producing fatty acid ester and genetically engineered for reduced activity of the polypeptide corresponding to YbaW, produces an increase in fatty acid esters of 1%, 2%, 3%, 4%, 5%, 10%, 15%, 20%, 25%, 30%, 40%, 50%, 60%, 70%, 80%, 90% or more compared to a similar recombinant cell that has not been genetically engineered for reduced activity of the polypeptide corresponding to YbaW.

In some aspects, the recombinant cell producing fatty acid ester and genetically engineered for reduced activity of the polypeptide corresponding to YbaW and combined with a Asch(V296A) variant, produces an increase in C10 fatty acid esters compared to a similar recombinant cell that has not been genetically engineered for reduced activity of the polypeptide corresponding to YbaW. In other aspects, the recombinant cell producing fatty acid ester and genetically engineered for reduced activity of the polypeptide corresponding to YbaW, produces an increase in C10 fatty acid esters of 1%, 2%, 3%, 4%, 5%, 10%, 15%, 20%, 25%, 30%, 40%, 50%, 60%, 70%, 80%, 90% or more compared to a similar recombinant cell that has not been genetically engineered for reduced activity of the polypeptide corresponding to YbaW.

In some aspects, a method for decreasing free fatty acid production comprises culturing a recombinant cell that produces fatty acid ester and is genetically engineered for reduced activity of a polypeptide encoded by a gene with an amino acid sequence set forth in SEQ ID NO: 2 and corresponds to the polypeptide designated as YbaW. In other aspects, the polypeptide encoded by a gene with an amino acid sequence designated as YbaW comprises a polypeptide having more than 50%, more than 55%, more than 60%, more than 65%, more than 70%, more than 80%, more than 85%, more than 90%, more than 91%, more than 92%, more than 93%, more than 94%, more than 95%, more than 96%, more than 97%, more than 98%, or more than 99% sequence identity to the amino acid sequence of SEQ ID NO: 2.

In some aspects, method for decreasing free fatty acid production comprises culturing the recombinant cell producing fatty acid ester and genetically engineered for reduced activity of the polypeptide corresponding to YbaW to produce a reduction in total free fatty acids compared to a similar recombinant cell that has not been genetically engineered for reduced activity of the polypeptide corresponding to YbaW. In other aspects, the recombinant cell producing fatty acid ester and genetically engineered for reduced activity of the polypeptide corresponding to YbaW, produces a reduction in total free fatty acids of 1%, 2%, 3%, 4%, 5%, 10%, 15%, 20%, 25%, 30%, 40%, 50%, 60%, 70%, 80%, 90% or more compared to a similar recombinant cell that has not been genetically engineered for reduced activity of the polypeptide corresponding to YbaW.

In some aspects, method for decreasing free fatty acid production comprises culturing the recombinant cell producing fatty acid ester and genetically engineered for reduced activity of the polypeptide corresponding to YbaW to produce a reduction in C10 free fatty acids compared to a similar recombinant cell that has not been genetically engineered for reduced activity of the polypeptide corresponding to YbaW. In other aspects, the recombinant cell producing fatty acid ester and genetically engineered for reduced activity of the polypeptide corresponding to YbaW, produces a reduction in C10 free fatty acids of 1%, 2%, 3%, 4%, 5%, 10%, 15%, 20%, 25%, 30%, 40%, 50%, 60%, 70%, 80%, 90% or more compared to a similar recombinant cell that has not been genetically engineered for reduced activity of the polypeptide corresponding to YbaW.

In some aspects, method for decreasing free fatty acid production comprises culturing the recombinant cell producing fatty acid ester and genetically engineered for reduced activity of the polypeptide corresponding to YbaW and combined with a Asch(V296A) variant to produce a reduction in C10 free fatty acids compared to a similar recombinant cell that has not been genetically engineered for reduced activity of the polypeptide corresponding to YbaW. In other aspects, the recombinant cell producing fatty acid ester and genetically engineered for reduced activity of the polypeptide corresponding to YbaW, produces a reduction in C10 free fatty acids of 1%, 2%, 3%, 4%, 5%, 10%, 15%, 20%, 25%, 30%, 40%, 50%, 60%, 70%, 80%, 90% or more compared to a similar recombinant cell that has not been genetically engineered for reduced activity of the polypeptide corresponding to YbaW.

In some aspects, a recombinant cell produces fatty acid ester and is genetically engineered for reduced activity of a polypeptide encoded by a gene with an amino acid sequence set forth in SEQ ID NO: 3 and corresponds to the polypeptide designated as FrmB. In other aspects, the polypeptide encoded by a gene with an amino acid sequence designated as FrmB comprises a polypeptide having more than 50%, more than 55%, more than 60%, more than 65%, more than 70%, more than 80%, more than 85%, more than 90%, more than 91%, more than 92%, more than 93%, more than 94%, more than 95%, more than 96%, more than 97%, more than 98%, or more than 99% sequence identity to the amino acid sequence of SEQ ID NO: 3.

In some aspects, the recombinant cell producing fatty acid ester and genetically engineered for reduced activity of the polypeptide corresponding to FrmB, produces a reduction in total free fatty acids compared to a similar recombinant cell that has not been genetically engineered for reduced activity of the polypeptide corresponding to FrmB. In other aspects, the recombinant cell producing fatty acid ester and genetically engineered for reduced activity of the polypeptide corresponding to FrmB, produces a reduction in total free fatty acids of 1%, 2%, 3%, 4%, 5%, 10%, 15%, 20%, 25%, 30%, 40%, 50%, 60%, 70%, 80%, 90% or more compared to a similar recombinant cell that has not been genetically engineered for reduced activity of the polypeptide corresponding to FrmB.

In some aspects, the recombinant cell producing fatty acid ester and genetically engineered for reduced activity of the polypeptide corresponding to FrmB, produces a reduction in C10 free fatty acids compared to a similar recombinant cell that has not been genetically engineered for reduced activity of the polypeptide corresponding to FrmB. In other aspects, the recombinant cell producing fatty acid ester and genetically engineered for reduced activity of the polypeptide corresponding to FrmB, produces a reduction in C10 free fatty acids of 1%, 2%, 3%, 4%, 5%, 10%, 15%, 20%, 25%, 30%, 40%, 50%, 60%, 70%, 80%, 90% or more compared to a similar recombinant cell that has not been genetically engineered for reduced activity of the polypeptide corresponding to FrmB.

In some aspects, the recombinant cell producing fatty acid ester and genetically engineered for reduced activity of the polypeptide corresponding to FrmB and combined with a Asch(V296A) variant, produces a reduction in C10 free fatty acids compared to a similar recombinant cell that has not been genetically engineered for reduced activity of the polypeptide corresponding to FrmB. In other aspects, the recombinant cell producing fatty acid ester and genetically engineered for reduced activity of the polypeptide corresponding to FrmB, produces a reduction in C10 free fatty acids of 1%, 2%, 3%, 4%, 5%, 10%, 15%, 20%, 25%, 30%, 40%, 50%, 60%, 70%, 80%, 90% or more compared to a similar recombinant cell that has not been genetically engineered for reduced activity of the polypeptide corresponding to FrmB.

In some aspects, the recombinant cell producing fatty acid ester and genetically engineered for reduced activity of the polypeptide corresponding to FrmB, produces a decreased ratio of total free fatty acids to fatty acid esters compared to a similar recombinant cell that has not been genetically engineered for reduced activity of the polypeptide corresponding to FrmB. In other aspects, the recombinant cell producing fatty acid ester and genetically engineered for reduced activity of the polypeptide corresponding to FrmB, produces a decreased ratio of total free fatty acids to fatty acid esters of 1%, 2%, 3%, 4%, 5%, 10%, 15%, 20%, 25%, 30%, 40%, 50%, 60%, 70%, 80%, 90% or more compared to a similar recombinant cell that has not been genetically engineered for reduced activity of the polypeptide corresponding to FrmB.

In some aspects, the recombinant cell producing fatty acid ester and genetically engineered for reduced activity of the polypeptide corresponding to FrmB, produces an increase in total fatty acid esters compared to a similar recombinant cell that has not been genetically engineered for reduced activity of the polypeptide corresponding to FrmB. In other aspects, the recombinant cell producing fatty acid ester and genetically engineered for reduced activity of the polypeptide corresponding to FrmB, produces an increase in fatty acid esters of 1%, 2%, 3%, 4%, 5%, 10%, 15%, 20%, 25%, 30%, 40%, 50%, 60%, 70%, 80%, 90% or more compared to a similar recombinant cell that has not been genetically engineered for reduced activity of the polypeptide corresponding to FrmB.

In some aspects, the recombinant cell producing fatty acid ester and genetically engineered for reduced activity of the polypeptide corresponding to FrmB and combined with a Asch(V296A) variant, produces an increase in C10 fatty acid esters compared to a similar recombinant cell that has not been genetically engineered for reduced activity of the polypeptide corresponding to FrmB. In other aspects, the recombinant cell producing fatty acid ester and genetically engineered for reduced activity of the polypeptide corresponding to FrmB, produces an increase in C10 fatty acid esters of 1%, 2%, 3%, 4%, 5%, 10%, 15%, 20%, 25%, 30%, 40%, 50%, 60%, 70%, 80%, 90% or more compared to a similar recombinant cell that has not been genetically engineered for reduced activity of the polypeptide corresponding to FrmB.

In some aspects, a method for decreasing free fatty acid production comprises culturing a recombinant cell that produces fatty acid ester and is genetically engineered for reduced activity of a polypeptide encoded by a gene with an amino acid sequence set forth in SEQ ID NO: 3 and corresponds to the polypeptide designated as FrmB. In other aspects, the polypeptide encoded by a gene with an amino acid sequence designated as FrmB comprises a polypeptide having more than 50%, more than 55%, more than 60%, more than 65%, more than 70%, more than 80%, more than 85%, more than 90%, more than 91%, more than 92%, more than 93%, more than 94%, more than 95%, more than 96%, more than 97%, more than 98%, or more than 99% sequence identity to the amino acid sequence of SEQ ID NO: 3.

In some aspects, method for decreasing free fatty acid production comprises culturing the recombinant cell producing fatty acid ester and genetically engineered for reduced activity of the polypeptide corresponding to FrmB to produce a reduction in total free fatty acids compared to a similar recombinant cell that has not been genetically engineered for reduced activity of the polypeptide corresponding to FrmB. In other aspects, the recombinant cell producing fatty acid ester and genetically engineered for reduced activity of the polypeptide corresponding to FrmB, produces a reduction in total free fatty acids of 1%, 2%, 3%, 4%, 5%, 10%, 15%, 20%, 25%, 30%, 40%, 50%, 60%, 70%, 80%, 90% or more compared to a similar recombinant cell that has not been genetically engineered for reduced activity of the polypeptide corresponding to FrmB.

In some aspects, method for decreasing free fatty acid production comprises culturing the recombinant cell producing fatty acid ester and genetically engineered for reduced activity of the polypeptide corresponding to FrmB to produce a reduction in C10 free fatty acids compared to a similar recombinant cell that has not been genetically engineered for reduced activity of the polypeptide corresponding to FrmB. In other aspects, the recombinant cell producing fatty acid ester and genetically engineered for reduced activity of the polypeptide corresponding to FrmB, produces a reduction in C10 free fatty acids of 1%, 2%, 3%, 4%, 5%, 10%, 15%, 20%, 25%, 30%, 40%, 50%, 60%, 70%, 80%, 90% or more compared to a similar recombinant cell that has not been genetically engineered for reduced activity of the polypeptide corresponding to FrmB.

In some aspects, method for decreasing free fatty acid production comprises culturing the recombinant cell producing fatty acid ester and genetically engineered for reduced activity of the polypeptide corresponding to FrmB and combined with a Asch(V296A) variant to produce a reduction in C10 free fatty acids compared to a similar recombinant cell that has not been genetically engineered for reduced activity of the polypeptide corresponding to FrmB. In other aspects, the recombinant cell producing fatty acid ester and genetically engineered for reduced activity of the polypeptide corresponding to FrmB, produces a reduction in C10 free fatty acids of 1%, 2%, 3%, 4%, 5%, 10%, 15%, 20%, 25%, 30%, 40%, 50%, 60%, 70%, 80%, 90% or more compared to a similar recombinant cell that has not been genetically engineered for reduced activity of the polypeptide corresponding to FrmB.

In some aspects, a recombinant cell produces fatty acid ester and is genetically engineered for reduced activity of a polypeptide encoded by a gene with an amino acid sequence set forth in SEQ ID NO: 4 and corresponds to the polypeptide designated as YdiI. In other aspects, the polypeptide encoded by a gene with an amino acid sequence designated as YdiI comprises a polypeptide having more than 50%, more than 55%, more than 60%, more than 65%, more than 70%, more than 80%, more than 85%, more than 90%, more than 91%, more than 92%, more than 93%, more than 94%, more than 95%, more than 96%, more than 97%, more than 98%, or more than 99% sequence identity to the amino acid sequence of SEQ ID NO: 4.

In some aspects, the recombinant cell producing fatty acid ester and genetically engineered for reduced activity of the polypeptide corresponding to YdiI, produces a reduction in total free fatty acids compared to a similar recombinant cell that has not been genetically engineered for reduced activity of the polypeptide corresponding to YdiI. In other aspects, the recombinant cell producing fatty acid ester and genetically engineered for reduced activity of the polypeptide corresponding to YdiI, produces a reduction in total free fatty acids of 1%, 2%, 3%, 4%, 5%, 10%, 15%, 20%, 25%, 30%, 40%, 50%, 60%, 70%, 80%, 90% or more compared to a similar recombinant cell that has not been genetically engineered for reduced activity of the polypeptide corresponding to YdiI.

In some aspects, the recombinant cell producing fatty acid ester and genetically engineered for reduced activity of the polypeptide corresponding to YdiI, produces a reduction in C8 and/or C10 free fatty acids compared to a similar recombinant cell that has not been genetically engineered for reduced activity of the polypeptide corresponding to YdiI. In other aspects, the recombinant cell producing fatty acid ester and genetically engineered for reduced activity of the polypeptide corresponding to YdiI, produces a reduction in C8 free fatty acids of 1%, 2%, 3%, 4%, 5%, 10%, 15%, 20%, 25%, 30%, 40%, 50%, 60%, 70%, 80%, 90% or more compared to a similar recombinant cell that has not been genetically engineered for reduced activity of the polypeptide corresponding to YdiI.

In some aspects, the recombinant cell producing fatty acid ester and genetically engineered for reduced activity of the polypeptide corresponding to YdiI and combined with a Asch (T184I, V268A, V296A) and/or Asch(V296A) variant, produces a reduction in C8 free fatty acids compared to a similar recombinant cell that has not been genetically engineered for reduced activity of the polypeptide corresponding to YdiI. In other aspects, the recombinant cell producing fatty acid ester and genetically engineered for reduced activity of the polypeptide corresponding to YdiI, produces a reduction in C8 free fatty acids of 1%, 2%, 3%, 4%, 5%, 10%, 15%, 20%, 25%, 30%, 40%, 50%, 60%, 70%, 80%, 90% or more compared to a similar recombinant cell that has not been genetically engineered for reduced activity of the polypeptide corresponding to YdiI.

In some aspects, the recombinant cell producing fatty acid ester and genetically engineered for reduced activity of the polypeptide corresponding to YdiI, produces a decreased ratio of total free fatty acids to fatty acid esters compared to a similar recombinant cell that has not been genetically engineered for reduced activity of the polypeptide corresponding to YdiI. In other aspects, the recombinant cell producing fatty acid ester and genetically engineered for reduced activity of the polypeptide corresponding to YdiI, produces a decreased ratio of total free fatty acids to fatty acid esters of 1%, 2%, 3%, 4%, 5%, 10%, 15%, 20%, 25%, 30%, 40%, 50%, 60%, 70%, 80%, 90% or more compared to a similar recombinant cell that has not been genetically engineered for reduced activity of the polypeptide corresponding to YdiI.

In some aspects, the recombinant cell producing fatty acid ester and genetically engineered for reduced activity of the polypeptide corresponding to YdiI, produces an increase in total fatty acid esters compared to a similar recombinant cell that has not been genetically engineered for reduced activity of the polypeptide corresponding to YdiI. In other aspects, the recombinant cell producing fatty acid ester and genetically engineered for reduced activity of the polypeptide corresponding to YdiI, produces an increase in fatty acid esters of 1%, 2%, 3%, 4%, 5%, 10%, 15%, 20%, 25%, 30%, 40%, 50%, 60%, 70%, 80%, 90% or more compared to a similar recombinant cell that has not been genetically engineered for reduced activity of the polypeptide corresponding to YdiI.

In some aspects, the recombinant cell producing fatty acid ester and genetically engineered for reduced activity of the polypeptide corresponding to YdiI and combined with a Asch (T184I, V268A, V296A) and/or Asch(V296A) variant, produces an increase in C8 fatty acid esters compared to a similar recombinant cell that has not been genetically engineered for reduced activity of the polypeptide corresponding to YdiI. In other aspects, the recombinant cell producing fatty acid ester and genetically engineered for reduced activity of the polypeptide corresponding to YdiI, produces an increase in C8 fatty acid esters of 1%, 2%, 3%, 4%, 5%, 10%, 15%, 20%, 25%, 30%, 40%, 50%, 60%, 70%, 80%, 90% or more compared to a similar recombinant cell that has not been genetically engineered for reduced activity of the polypeptide corresponding to YdiI.

In some aspects, a method for decreasing free fatty acid production comprises culturing a recombinant cell that produces fatty acid ester and is genetically engineered for reduced activity of a polypeptide encoded by a gene with an amino acid sequence set forth in SEQ ID NO: 4 and corresponds to the polypeptide designated as YdiI. In other aspects, the polypeptide encoded by a gene with an amino acid sequence designated as YdiI comprises a polypeptide having more than 50%, more than 55%, more than 60%, more than 65%, more than 70%, more than 80%, more than 85%, more than 90%, more than 91%, more than 92%, more than 93%, more than 94%, more than 95%, more than 96%, more than 97%, more than 98%, or more than 99% sequence identity to the amino acid sequence of SEQ ID NO: 4.

In some aspects, method for decreasing free fatty acid production comprises culturing the recombinant cell producing fatty acid ester and genetically engineered for reduced activity of the polypeptide corresponding to YdiI to produce a reduction in total free fatty acids compared to a similar recombinant cell that has not been genetically engineered for reduced activity of the polypeptide corresponding to YdiI. In other aspects, the recombinant cell producing fatty acid ester and genetically engineered for reduced activity of the polypeptide corresponding to YdiI, produces a reduction in total free fatty acids of 1%, 2%, 3%, 4%, 5%, 10%, 15%, 20%, 25%, 30%, 40%, 50%, 60%, 70%, 80%, 90% or more compared to a similar recombinant cell that has not been genetically engineered for reduced activity of the polypeptide corresponding to YdiI.

In some aspects, a method for decreasing free fatty acid production comprises culturing the recombinant cell producing fatty acid ester and genetically engineered for reduced activity of the polypeptide corresponding to YdiI to produce a reduction in C8 free fatty acids compared to a similar recombinant cell that has not been genetically engineered for reduced activity of the polypeptide corresponding to YdiI. In other aspects, the recombinant cell producing fatty acid ester and genetically engineered for reduced activity of the polypeptide corresponding to YdiI, produces a reduction in C8 free fatty acids of 1%, 2%, 3%, 4%, 5%, 10%, 15%, 20%, 25%, 30%, 40%, 50%, 60%, 70%, 80%, 90% or more compared to a similar recombinant cell that has not been genetically engineered for reduced activity of the polypeptide corresponding to YdiI.

In some aspects, a method for decreasing free fatty acid production comprises culturing the recombinant cell producing fatty acid ester and genetically engineered for reduced activity of the polypeptide corresponding to YdiI and combined with a Asch (T184I, V268A, V296A) and/or Asch(V296A) variant to produce a reduction in C8 free fatty acids compared to a similar recombinant cell that has not been genetically engineered for reduced activity of the polypeptide corresponding to YdiI. In other aspects, the recombinant cell producing fatty acid ester and genetically engineered for reduced activity of the polypeptide corresponding to YdiI, produces a reduction in C8 free fatty acids of 1%, 2%, 3%, 4%, 5%, 10%, 15%, 20%, 25%, 30%, 40%, 50%, 60%, 70%, 80%, 90% or more compared to a similar recombinant cell that has not been genetically engineered for reduced activity of the polypeptide corresponding to YdiI.

In some aspects, a recombinant cell produces fatty acid ester and is genetically engineered for reduced activity of a polypeptide encoded by a gene with an amino acid sequence set forth in SEQ ID NO: 4 and corresponds to the polypeptide designated as YdiI and is genetically engineered for reduced activity of a polypeptide encoded by a gene with an amino acid sequence set forth in SEQ ID NO: 5 and corresponds to the polypeptide designated as BioH. In other aspects, the polypeptide encoded by a gene with an amino acid sequence designated as YdiI comprises a polypeptide having more than 50%, more than 55%, more than 60%, more than 65%, more than 70%, more than 80%, more than 85%, more than 90%, more than 91%, more than 92%, more than 93%, more than 94%, more than 95%, more than 96%, more than 97%, more than 98%, or more than 99% sequence identity to the amino acid sequence of SEQ ID NO: 4. In other aspects, the polypeptide encoded by a gene with an amino acid sequence designated as BioH comprises a polypeptide having more than 50%, more than 55%, more than 60%, more than 65%, more than 70%, more than 80%, more than 85%, more than 90%, more than 91%, more than 92%, more than 93%, more than 94%, more than 95%, more than 96%, more than 97%, more than 98%, or more than 99% sequence identity to the amino acid sequence of SEQ ID NO: 5.

In some aspects, the recombinant cell producing fatty acid ester and genetically engineered for reduced activity of the polypeptide corresponding to YdiI and for the polypeptide corresponding to BioH, produces a reduction in total free fatty acids compared to a similar recombinant cell that has not been genetically engineered for reduced activity of the polypeptides corresponding to YdiI and BioH. In other aspects, the recombinant cell producing fatty acid ester and genetically engineered for reduced activity of the polypeptides corresponding to YdiI and BioH, produces a reduction in total free fatty acids of 1%, 2%, 3%, 4%, 5%, 10%, 15%, 20%, 25%, 30%, 40%, 50%, 60%, 70%, 80%, 90% or more compared to a similar recombinant cell that has not been genetically engineered for reduced activity of the polypeptides corresponding to YdiI and BioH.

In some aspects, the recombinant cell producing fatty acid ester and genetically engineered for reduced activity of the polypeptides corresponding to YdiI and BioH, produces a reduction in C8 and/or C10 free fatty acids compared to a similar recombinant cell that has not been genetically engineered for reduced activity of the polypeptides corresponding to YdiI and BioH. In other aspects, the recombinant cell producing fatty acid ester and genetically engineered for reduced activity of the polypeptides corresponding to YdiI and BioH, produces a reduction in C8 and/or C10 free fatty acids of 1%, 2%, 3%, 4%, 5%, 10%, 15%, 20%, 25%, 30%, 40%, 50%, 60%, 70%, 80%, 90% or more compared to a similar recombinant cell that has not been genetically engineered for reduced activity of the polypeptides corresponding to YdiI and BioH.

In some aspects, a recombinant cell produces fatty acid ester and is genetically engineered for reduced activity of a polypeptides encoded by a gene with an amino acid sequence set forth in SEQ ID NO: 4 and corresponding to the polypeptide designated as YdiI, a polypeptide encoded by a gene with an amino acid sequence set forth in SEQ ID NO: 5 and corresponding to the polypeptide designated as BioH, and a polypeptide encoded by a gene with an amino acid sequence set forth in SEQ ID NO: 6 and corresponding to the polypeptide designated as TesA. In other aspects, the polypeptide encoded by a gene with an amino acid sequence designated as YdiI comprises a polypeptide having more than 50%, more than 55%, more than 60%, more than 65%, more than 70%, more than 80%, more than 85%, more than 90%, more than 91%, more than 92%, more than 93%, more than 94%, more than 95%, more than 96%, more than 97%, more than 98%, or more than 99% sequence identity to the amino acid sequence of SEQ ID NO: 4. In other aspects, the polypeptide encoded by a gene with an amino acid sequence designated as BioH comprises a polypeptide having more than 50%, more than 55%, more than 60%, more than 65%, more than 70%, more than 80%, more than 85%, more than 90%, more than 91%, more than 92%, more than 93%, more than 94%, more than 95%, more than 96%, more than 97%, more than 98%, or more than 99% sequence identity to the amino acid sequence of SEQ ID NO: 5. In other aspects, the polypeptide encoded by a gene with an amino acid sequence designated as TesA comprises a polypeptide having more than 50%, more than 55%, more than 60%, more than 65%, more than 70%, more than 80%, more than 85%, more than 90%, more than 91%, more than 92%, more than 93%, more than 94%, more than 95%, more than 96%, more than 97%, more than 98%, or more than 99% sequence identity to the amino acid sequence of SEQ ID NO: 6.

In some aspects, the recombinant cell producing fatty acid ester and genetically engineered for reduced activity of the polypeptides corresponding to YdiI, BioH, and TesA, produces a reduction in total free fatty acids compared to a similar recombinant cell that has not been genetically engineered for reduced activity of the polypeptides corresponding to YdiI, BioH, and TesA. In other aspects, the recombinant cell producing fatty acid ester and genetically engineered for reduced activity of the polypeptides corresponding to YdiI, BioH, and TesA, produces a reduction in total free fatty acids of 1%, 2%, 3%, 4%, 5%, 10%, 15%, 20%, 25%, 30%, 40%, 50%, 60%, 70%, 80%, 90% or more compared to a similar recombinant cell that has not been genetically engineered for reduced activity of the polypeptides corresponding to YdiI, BioH, and TesA.

In some aspects, the recombinant cell producing fatty acid ester and genetically engineered for reduced activity of the polypeptides corresponding to YdiI, BioH, and TesA, produces a reduction in C8 and/or C10 free fatty acids compared to a similar recombinant cell that has not been genetically engineered for reduced activity of the polypeptides corresponding to YdiI, BioH, TesA. In other aspects, the recombinant cell producing fatty acid ester and genetically engineered for reduced activity of the polypeptides corresponding to YdiI, BioH, and TesA, produces a reduction in C8 and/or C10 free fatty acids of 1%, 2%, 3%, 4%, 5%, 10%, 15%, 20%, 25%, 30%, 40%, 50%, 60%, 70%, 80%, 90% or more compared to a similar recombinant cell that has not been genetically engineered for reduced activity of the polypeptides corresponding to YdiI, BioH, and TesA.

In some aspects, the recombinant cell producing fatty acid ester and genetically engineered for reduced activity of the polypeptides corresponding to YdiI, BioH, and TesA, produces an increase in total fatty acid esters compared to a similar recombinant cell that has not been genetically engineered for reduced activity of the polypeptides corresponding to YdiI, BioH, and TesA. In other aspects, the recombinant cell producing fatty acid ester and genetically engineered for reduced activity of the polypeptide corresponding to YdiI, BioH, and TesA, produces an increase in fatty acid esters of 1%, 2%, 3%, 4%, 5%, 10%, 15%, 20%, 25%, 30%, 40%, 50%, 60%, 70%, 80%, 90% or more compared to a similar recombinant cell that has not been genetically engineered for reduced activity of the polypeptides corresponding to YdiI, BioH, and TesA.

In some aspects, the recombinant cell producing fatty acid ester and genetically engineered for reduced activity of the polypeptides corresponding to YdiI, BioH, and TesA, produces an increase in C8 fatty acid esters compared to a similar recombinant cell that has not been genetically engineered for reduced activity of the polypeptides corresponding to YdiI, BioH, and TesA. In other aspects, the recombinant cell producing fatty acid ester and genetically engineered for reduced activity of the polypeptide corresponding to YdiI, BioH, and TesA, produces an increase in C8 fatty acid esters of 1%, 2%, 3%, 4%, 5%, 10%, 15%, 20%, 25%, 30%, 40%, 50%, 60%, 70%, 80%, 90% or more compared to a similar recombinant cell that has not been genetically engineered for reduced activity of the polypeptides corresponding to YdiI, BioH, and TesA.

In some aspects, the recombinant cell producing fatty acid ester and genetically engineered for reduced activity of the polypeptides corresponding to YdiI, BioH, and TesA, produces an increase in total fatty acid esters compared to a similar recombinant cell that has not been genetically engineered for reduced activity of the polypeptides corresponding to YdiI, BioH, and TesA. In other aspects, the recombinant cell producing fatty acid ester and genetically engineered for reduced activity of the polypeptide corresponding to YdiI, BioH, and TesA, produces an increase in fatty acid esters of 1%, 2%, 3%, 4%, 5%, 10%, 15%, 20%, 25%, 30%, 40%, 50%, 60%, 70%, 80%, 90% or more compared to a similar recombinant cell that has not been genetically engineered for reduced activity of the polypeptides corresponding to YdiI, BioH, and TesA.

In some aspects, the recombinant cell producing fatty acid ester and genetically engineered for reduced activity of the polypeptides corresponding to YdiI, BioH, and TesA, produces an increase in C8 fatty acid esters compared to a similar recombinant cell that has not been genetically engineered for reduced activity of the polypeptides corresponding to YdiI, BioH, and TesA. In other aspects, the recombinant cell producing fatty acid ester and genetically engineered for reduced activity of the polypeptide corresponding to YdiI, BioH, and TesA, produces an increase in C8 fatty acid esters of 1%, 2%, 3%, 4%, 5%, 10%, 15%, 20%, 25%, 30%, 40%, 50%, 60%, 70%, 80%, 90% or more compared to a similar recombinant cell that has not been genetically engineered for reduced activity of the polypeptides corresponding to YdiI, BioH, and TesA.

In some aspects, the recombinant cell producing fatty acid ester and genetically engineered for reduced activity of the polypeptides corresponding to YdiI, BioH, and TesA, produces a decreased ratio of total free fatty acids to fatty acid esters compared to a similar recombinant cell that has not been genetically engineered for reduced activity of the polypeptides corresponding to YdiI, BioH, and TesA. In other aspects, the recombinant cell producing fatty acid ester and genetically engineered for reduced activity of the polypeptides corresponding to YdiI, BioH, and TesA, produces a decreased ratio of total fatty acid esters to free fatty acids of 1%, 2%, 3%, 4%, 5%, 10%, 15%, 20%, 25%, 30%, 40%, 50%, 60%, 70%, 80%, 90% or more compared to a similar recombinant cell that has not been genetically engineered for reduced activity of the polypeptide corresponding to YdiI, BioH, and TesA.

In some aspects, the recombinant cell producing fatty acid ester and genetically engineered for reduced activity of the polypeptides corresponding to YdiI, BioH, and TesA, produces a decreased ratio of total free fatty acids to fatty acid esters compared to a similar recombinant cell that has not been genetically engineered for reduced activity of the polypeptides corresponding to YdiI, BioH, and TesA. In other aspects, the recombinant cell producing fatty acid ester and genetically engineered for reduced activity of the polypeptides corresponding to YdiI, BioH, and TesA, produces a decreased ratio of total free fatty acids to fatty acid esters of 1%, 2%, 3%, 4%, 5%, 10%, 15%, 20%, 25%, 30%, 40%, 50%, 60%, 70%, 80%, 90% or more compared to a similar recombinant cell that has not been genetically engineered for reduced activity of the polypeptide corresponding to YdiI, BioH, and TesA.

In some aspects, a recombinant cell produces fatty acid ester and is genetically engineered for increased activity of a polypeptide encoded by a gene with an amino acid sequence set forth in SEQ ID NO: 7 and corresponds to the polypeptide designated as ACS. In other aspects, the polypeptide encoded by a gene with an amino acid sequence designated as ACS comprises a polypeptide having more than 50%, more than 55%, more than 60%, more than 65%, more than 70%, more than 80%, more than 85%, more than 90%, more than 91%, more than 92%, more than 93%, more than 94%, more than 95%, more than 96%, more than 97%, more than 98%, or more than 99% sequence identity to the amino acid sequence of SEQ ID NO: 7.

In some aspects, the recombinant cell producing fatty acid ester and genetically engineered for increased activity of the polypeptide corresponding to ACS, produces a reduction in total free fatty acids compared to a similar recombinant cell that has not been genetically engineered for increased activity of the polypeptide corresponding to ACS. In other aspects, the recombinant cell producing fatty acid ester and genetically engineered for increased activity of the polypeptide corresponding to ACS, produces a reduction in total free fatty acids of 1%, 2%, 3%, 4%, 5%, 10%, 15%, 20%, 25%, 30%, 40%, 50%, 60%, 70%, 80%, 90% or more compared to a similar recombinant cell that has not been genetically engineered for increased activity of the polypeptide corresponding to ACS.

In some aspects, the recombinant cell producing fatty acid ester and genetically engineered for increased activity of the polypeptide corresponding to ACS, produces a reduction in C8 free fatty acids compared to a similar recombinant cell that has not been genetically engineered for increased activity of the polypeptide corresponding to ACS. In other aspects, the recombinant cell producing fatty acid ester and genetically engineered for increased activity of the polypeptide corresponding to ACS, produces a reduction in C8 free fatty acids of 1%, 2%, 3%, 4%, 5%, 10%, 15%, 20%, 25%, 30%, 40%, 50%, 60%, 70%, 80%, 90% or more compared to a similar recombinant cell that has not been genetically engineered for increased activity of the polypeptide corresponding to ACS.

In some aspects, the recombinant cell producing fatty acid ester and genetically engineered for increased activity of the polypeptide corresponding to ACS and combined with a Asch (T184I, F236L, V268A, V296A, V317A, S328G) variant, produces a reduction in C8 free fatty acids compared to a similar recombinant cell that has not been genetically engineered for increased activity of the polypeptide corresponding to ACS. In other aspects, the recombinant cell producing fatty acid ester and genetically engineered for increased activity of the polypeptide corresponding to ACS, produces a reduction in C8 free fatty acids of 1%, 2%, 3%, 4%, 5%, 10%, 15%, 20%, 25%, 30%, 40%, 50%, 60%, 70%, 80%, 90% or more compared to a similar recombinant cell that has not been genetically engineered for increased activity of the polypeptide corresponding to ACS.

In some aspects, the recombinant cell producing fatty acid ester and genetically engineered for increased activity of the polypeptide corresponding to ACS, produces a decreased ratio of total free fatty acids to fatty acid esters compared to a similar recombinant cell that has not been genetically engineered for increased activity of the polypeptide corresponding to ACS. In other aspects, the recombinant cell producing fatty acid ester and genetically engineered for increased activity of the polypeptide corresponding to ACS, produces a decreased ratio of total free fatty acids to fatty acid esters of 1%, 2%, 3%, 4%, 5%, 10%, 15%, 20%, 25%, 30%, 40%, 50%, 60%, 70%, 80%, 90% or more compared to a similar recombinant cell that has not been genetically engineered for increased activity of the polypeptide corresponding to ACS.

In some aspects, a recombinant cell produces fatty acid ester and is genetically engineered for increased activity of a polypeptide encoded by a gene with an amino acid sequence set forth in SEQ ID NO: 7 and corresponds to the polypeptide designated as ACS and for reduced activity of a polypeptide encoded by a gene with an amino acid sequence set forth in SEQ ID NO: 4 and corresponds to the polypeptide designated as YdiI and is genetically engineered for reduced activity of a polypeptide encoded by a gene with an amino acid sequence set forth in SEQ ID NO: 5 and corresponds to the polypeptide designated as BioH. In other aspects, the polypeptide encoded by a gene with an amino acid sequence designated as ACS comprises a polypeptide having more than 50%, more than 55%, more than 60%, more than 65%, more than 70%, more than 80%, more than 85%, more than 90%, more than 91%, more than 92%, more than 93%, more than 94%, more than 95%, more than 96%, more than 97%, more than 98%, or more than 99% sequence identity to the amino acid sequence of SEQ ID NO: 7.

In some aspects, the recombinant cell producing fatty acid ester and genetically engineered for increased activity of the polypeptide corresponding to ACS and decreased activity the polypeptides corresponding to YdiI and BioH, produces a reduction in total free fatty acids compared to a similar recombinant cell that has not been genetically engineered for increased activity of the polypeptide corresponding to ACS and decreased activity for the polypeptides corresponding to YdiI and BioH. In other aspects, the recombinant cell producing fatty acid ester and genetically engineered for increased activity of the polypeptide corresponding to ACS and decreased activity for the polypeptides corresponding to YdiI and BioH, produces a reduction in total free fatty acids of 1%, 2%, 3%, 4%, 5%, 10%, 15%, 20%, 25%, 30%, 40%, 50%, 60%, 70%, 80%, 90% or more compared to a similar recombinant cell that has not been genetically engineered for increased activity of the polypeptide corresponding to ACS and decreased activity for the polypeptides corresponding to YdiI and BioH.

In some aspects, the recombinant cell producing fatty acid ester and genetically engineered for increased activity of the polypeptide corresponding to ACS and decreased activity for the polypeptides corresponding to YdiI and BioH, produces a reduction in C8 and/or C10 free fatty acids compared to a similar recombinant cell that has not been genetically engineered for increased activity of the polypeptide corresponding to ACS and decreased activity for the polypeptides corresponding to YdiI and BioH. In other aspects, the recombinant cell producing fatty acid ester and genetically engineered for increased activity of the polypeptide corresponding to ACS and decreased activity for the polypeptides corresponding to YdiI and BioH, produces a reduction in C8 and/or C10 free fatty acids of 1%, 2%, 3%, 4%, 5%, 10%, 15%, 20%, 25%, 30%, 40%, 50%, 60%, 70%, 80%, 90% or more compared to a similar recombinant cell that has not been genetically engineered for increased activity of the polypeptide corresponding to ACS and decreased activity for the polypeptides corresponding to YdiI and BioH.

In some aspects, the recombinant cell producing fatty acid ester and genetically engineered for increased activity of the polypeptide corresponding to ACS and decreased activity for the polypeptides corresponding to YdiI and BioH and combined with a Asch (T184I, F236L, V268A, V296A, V317A, S328G) variant or a Asch (V296A) variant, produces a reduction in C8 and/or C10 free fatty acids compared to a similar recombinant cell that has not been genetically engineered for increased activity of the polypeptide corresponding to ACS and decreased activity for the polypeptides corresponding to YdiI and BioH. In other aspects, the recombinant cell producing fatty acid ester and genetically engineered for increased activity of the polypeptide corresponding to ACS and decreased activity for the polypeptides corresponding to YdiI and BioH, produces a reduction in C8 and/or C10 free fatty acids of 1%, 2%, 3%, 4%, 5%, 10%, 15%, 20%, 25%, 30%, 40%, 50%, 60%, 70%, 80%, 90% or more compared to a similar recombinant cell that has not been genetically engineered for increased activity of the polypeptide corresponding to ACS and decreased activity for the polypeptides corresponding to YdiI and BioH.

In some aspects, the recombinant cell producing fatty acid ester and genetically engineered for increased activity of the polypeptide corresponding to ACS and decreased activity for the polypeptides corresponding to YdiI and BioH, produces an increase in C8 fatty acid esters compared to a similar recombinant cell that has not been genetically engineered for increased activity of the polypeptide corresponding to ACS and decreased activity for the polypeptides corresponding to YdiI and BioH. In other aspects, the recombinant cell producing fatty acid ester and genetically engineered for increased activity of the polypeptide corresponding to ACS and decreased activity for the polypeptides corresponding to YdiI and BioH, produces an increase in C8 fatty acid esters of 1%, 2%, 3%, 4%, 5%, 10%, 15%, 20%, 25%, 30%, 40%, 50%, 60%, 70%, 80%, 90% or more compared to a similar recombinant cell that has not been genetically engineered for increased activity of the polypeptide corresponding to ACS and decreased activity for the polypeptides corresponding to YdiI and BioH.

### EXAMPLES

The following examples are provided to illustrate the disclosure, but are not intended to limit the scope thereof. All parts and percentages are by weight unless otherwise indicated.

### Example 1

The following recombinant E. coli strains were used in the following examples as indicated.

Recombinant Strain 1 is a mutant of the E. coli strain designated BW25113, available from the E. coli Genetic Strain Center (CGSC #7636; Dept. of Molecular, Cellular, and Developmental Biology, Yale University, New Haven, Conn.), having the following additional genetic modifications:

| Designation | Description |
|---|---|
| ΔldhA::frt | Deletion of native lactate dehydrogenase |
| ΔpflB::frt | Deletion of native pyruvate formate lyase |
| ΔmgsA::frt | Deletion of native methylglyoxal synthase |
| ΔpoxB::frt | Deletion of native pyruvate oxidase |
| Δpta-ack::frt | Deletion of native phosphotransacetylase and acetate kinase |
| Δtig:frt | Deletion of native trigger factor protein |
| ΔatoDAEB::frt | Deletion to disrupt short-chain poly-(R)-3-hydroxybutyrate synthesis |
| ΔfadD::frt | Deletion of native acyl-CoA synthase |
| ΔtesB::frt | Deletion of native thioesterase |
| ΔyciA::frt | Deletion of native thioesterase |
| ΔadhE | Deletion of native aldehyde-alcohol dehydrogenase |
| P_{pstSIH}-nphT7-ter-TT-loxP | Insertion of a heterologous gene encoding for Streptomyces Sp. CL190 |
| | acetoacetyl-CoA synthase (NphT7, SEQ ID NO: 14) and a heterologous |
| | gene encoding for Treponema denticola enoyl-CoA reductase (ter, SEQ ID |
| | NO: 13) under control of E. coli pstSIH promoter and an E. coli terminator |
| | at locus of native adhE gene |

Recombinant E. coli strains were prepared by using standard electroporation methods to transform Recombinant Strain 1 with one of the following recombinant plasmids as indicated.

Type 1 plasmids are pACYC plasmids containing the p15a origin of replication and a chloramphenicol resistance marker. These plasmid includes an operon encoding for a mutated Streptomyces sp. nphT7 gene encoding for a 3-ketoacyl-CoA synthase having H100L, I147S, F217V and S323A mutations (the “LSVA” NphT7 mutant, SEQ ID NO: 16), fadB (SEQ ID NO:8), and ter (SEQ ID NO: 13) under a native E. coli pstSIH promoter (SEQ ID NO: 24). This plasmid also includes an operon encoding for an Asch variant with a His-tagged Hche (SEQ ID NO: 22) under an E. coli phoE promoter (SEQ ID NO: 25). Finally, this plasmid also includes a cassette encoding for ACC (acetyl-CoA carboxylase). This ACC cassette contains an ACC (acetyl-CoA carboxylase) cassette including fused E. coli accD and accA genes (SEQ ID NO: 23) under a E. coli tpiA promoter (SEQ ID NO: 26), a cassette including the E. coli accB gene (SEQ ID NO: 27), and the E. coli accC gene (SEQ ID NO: 28), both under an E. coli rpiA promoter (SEQ ID NO: 29).

Type 2 plasmids are pET plasmids containing a ColE1 origin of replication and a kanamycin resistance marker. These plasmids include the mouse ACSM1 gene (with the mitochondrial leader sequence removed)(SEQ ID NO:30) with various promoter sequences and an E. coli terminator. Some plasmids include a constitutive promoter. Other plasmids include a promoter that is induced in response to lowering phosphate concentrations in the growth media.

Type 3A plasmids are pACYC plasmids containing the p15a origin of replication and a chloramphenicol resistance marker. These plasmids include an operon containing an E. coli bifunctional 3-hydroxyacyl-CoA dehydrogenase/dehydratase (fadB) gene (SEQ ID NO: 8) and a T. denticola enoyl-CoA (ter) gene (SEQ ID NO: 13) cassette, all under a native E. coli pstSIH promoter (SEQ ID NO: 24) and a native E. coli terminator. These plasmids also contains a Hahella chejuensis ester synthase gene (SEQ ID NO: 22) fused to a DNA sequence encoding a protein fragment containing 6 histidine residues and a protease recognition site under an E. coli phoE promoter (SEQ ID NO: 25). These plasmids also contain an ACC (acetyl-CoA carboxylase) cassette including fused E. coli accD and accA genes (SEQ ID NO: 23) under a E. coli tpiA promoter (SEQ ID NO: 26), a cassette including the E. coli accB (SEQ ID NO: 27), and E. coli accC genes (SEQ ID NO: 28) under an E. coli rpiA promoter (SEQ ID NO: 29).

Type 3B plasmids are pACYC plasmids containing the p15a origin of replication and a chloramphenicol resistance marker. These plasmids include an operon containing a mutated Streptomyces sp. nphT7 gene encoding for a 3-ketoacyl-CoA synthase having H100L, I147S, F217V and S323A mutations (the “LSVA” NphT7 mutant, SEQ ID NO: 15), an E. coli bifunctional 3-hydroxyacyl-CoA dehydrogenase/dehydratase (fadB) gene (SEQ ID NO: 8), and a T. denticola enoyl-CoA (ter) gene (SEQ ID NO: 13) cassette, all under a native E. coli pstSIH promoter (SEQ ID NO: 24) and a native E. coli terminator. These plasmids also contains a Hahella chejuensis ester synthase gene (SEQ ID NO: 22) fused to a DNA sequence encoding a protein fragment containing 6 histidine residues and a protease recognition site under an E. coli phoE promoter (SEQ ID NO: 25). These plasmids also contains an ACC (acetyl-CoA carboxylase) cassette including fused E. coli accD and accA genes (SEQ ID NO: 23) under a E. coli tpiA promoter (SEQ ID NO: 26), a cassette including the E. coli accB (SEQ ID NO: 27), and E. coli accC genes (SEQ ID NO: 28) under an E. coli rpiA promoter (SEQ ID NO: 29).

Type 4 plasmids are pET plasmids containing a ColE1 origin of replication and a kanamycin resistance marker. These plasmids include a Clostridiales bacterium 1_7_47_FAA 3-ketoacyl-CoA synthase gene variant (V223A, I246L) (SEQ ID NO: 31) fused to a DNA sequence encoding a protein fragment containing 6 histidine residues and a protease recognition site under an E. coli pSTSIH promoter (SEQ ID NO: 24).

### Small Scale Fermentation Method—Shake Flask Protocol

A culture of synthetic medium containing salts, glucose, NH₄Cl, and supplemented with vitamins, yeast extract, kanamycin and/or chloramphenicol (antibiotic added based on plasmids present in strain) is inoculated with the strain to be tested and grown for 24 hours at 32° C. and 300 RPM (rotations per minute). An OD₆₀₀ of a 1:10 dilution of this culture is determined in order to inoculate a production flask to a final OD₆₀₀ of 0.025. The production flasks contain 25 ml of synthetic medium containing salts and magnesium sulfate, and supplemented with vitamins, kanamycin and/or chloramphenicol (based on plasmids present in host strain), 2.5 mM phosphate, 6 g/l glucose, and 6-15 g/l glycerol. This synthetic medium represents a limited phosphate medium that promotes the activity of a low phosphate inducible promoter such as the E. coli PphoE promoter upon depletion of the phosphate. Production flasks are incubated at 32° C. and 300 RPM. When phosphate in the synthetic medium has depleted, 2 ml methyl myristate and 1 ml 12.5% TWEEN® 80 are added. The production assay is initiated by adding methanol (2% final concentration) and carbon (25-30 g/l glycerol or 30-50 g/l glucose final concentrations). The production flasks are incubated at 35° C. for 24 hours. At the end of the 24 hours, samples are taken from each production flask and additional methanol (0.25 ml) and carbon (15 g/l glycerol or 30-50 g/l glucose final concentration) are added to each production flask. The production flasks are then incubated at 35° C. for another 24 hours. At the end of the 48 hours, samples are taken from each production flask. Samples from the 24 hour and 48 hour time points are extracted with 0.2% HCl in MTBE (Methyl tert butyl ether) and the extracts are analyzed for free fatty acids and fatty acid esters by gas chromatography.

### Fed Batch Fermentation Method

A Seed 1 flask is prepared with 50 ml of synthetic medium. The synthetic medium comprises salts, glucose, ammonium sulfate, vitamins, yeast extract, and appropriate antibiotic (e.g., 20 μg/ml chloramphenicol). The Seed 1 flask is inoculated with the strain to be tested and grown overnight at 32° C. with shaking. A Seed 2 flask is then prepared with 50 ml of synthetic medium supplemented with 2% (w/v) methanol. The OD₆₀₀ of a 1:10 dilution of the Seed 1 flask is then determined and the amount of Seed 1 flask needed to inoculate the Seed 2 flask to a final OD₆₀₀ of 0.3 is calculated. The Seed 2 flask is then inoculated to a final OD₆₀₀ of 0.3 using the calculated amount of Seed 1 flask. The Seed 2 flask is then incubated at 32° C. with shaking for 6-7 hours.

Fed batch fermentation is performed in a New Brunswick fermenter equipped with a 2 L capacity vessel using an initial culture volume of 1.0 L. Fed batch cultures are grown in a chemically defined medium containing, per liter: 4.0 g ammonium sulfate, 3.6 g magnesium sulfate heptahydrate, 3.15 g citric acid, 2.0 g monobasic potassium phosphate, 0.42 g choline chloride, 0.29 g calcium chloride dihydrate, 0.06 g ferric chloride hexahydrate, 0.02 g cupric sulfate pentahydrate, 0.002 g manganese chloride tetrahydrate, 0.001 g zinc chloride, 0.015 g thiamine hydrochloride, 0.005 g biotin, 0.02 chloramphenicol, 0.1 g Sigma 204 antifoam, 20 g methanol, and 30 g glucose.

A fed batch fermentation culture is inoculated to an initial OD₆₀₀ of 0.002 from a Seed 2 shake flask. The fed batch fermentation culture is grown at 32° C. with pH maintained at pH 6.8 using ammonium hydroxide as the base titrant, with 1200 RPM of agitation, and with 1.5 standard liters per minute (slpm) of aeration. Once the initial batch glucose has depleted, the fermentation continues and the fed batch fermentation culture is constantly fed a glucose/methanol feed solution at a rate of 2 grams of glucose per liter of initial culture volume per hour for a period of six hours. The glucose/methanol feed solution contains 600 g glucose and 65 g methanol per liter.

Three hours after the initial batch glucose has depleted, the temperature is increased to 35° C. over a 10 minute period. After the temperature has reached 35° C., the base titrant is changed to sodium carbonate, the acid titrant is changed to sulfuric acid, and the pH is maintained below pH 7.2. 80 g of methyl myristate is then added to the fed batch culture.

Six hours after the initial batch glucose has depleted, the aeration is decreased to 0.5 slpm over a one hour period and the agitation is decreased to 1000 RPM over a three hour period. The agitation is then maintained at 1000 RPM until 27 h after the initial batch glucose has depleted. At 27 h after the initial batch glucose has depleted, the agitation is decreased to 800 RPM over a period of 20 hours.

Six hours after the initial batch glucose has depleted, the constant feed of the glucose/methanol feed solution is ceased. A feed of the glucose/methanol feed solution controlled by a modified pH-stat control system is then initiated. Under the modified pH-stat control system, each time the pH rises above pH 7.0, the glucose/methanol feed solution is delivered at a rate of 15 g glucose per liter of initial volume per hour for a period of 15 min and then halted. If the pH rises above pH 7.15, delivery of the glucose/methanol feed solution is ceased until the pH falls below 7.15. If the pH rises above pH 7.15 and the specific oxygen uptake rate of the fed batch fermentation culture falls below 2.6 mmol of oxygen per gram of dry cell weight per hour, the glucose/methanol feed solution is delivered at a rate of 15 g glucose per liter of initial volume per hour until the pH falls below 7.15.

Samples of the fed batch fermentation culture are taken at regular intervals. The samples are extracted with 0.2% HCL in MTBE (Methyl tert butyl ether) and assayed for fatty acid esters and free fatty acids by gas chromatography. Tables A1 and A2 shows an outline of a fed batch fermentation.

TABLE A1

| | |
|---|---|
| Time | Event |

| — | | Grow Seed 1 flask. |
|---|---|---|
| — | | Grow Seed 2 flask. |
| — | | Inoculate fed batch culture. |
| Fermentation | | Growth under initial batch glucose at 32° C. with pH |
| begins | | maintained at pH 6.8 using ammonium hydroxide as |
| | | the base titrant, with 1200 RPM of agitation, and with |
| | | 1.5 standard liters per minute (slpm) of aeration. |
| Initial batch | | Feed glucose/methanol feed solution at 2 g of glucose |
| glucose depletion | | per liter of initial culture per hour. |
| 3 | h post depletion | Temperature is increased to 35° C. over a 10 minute |
| | | period. After the temperature has reached 35° C., the |
| | | base titrant is changed to sodium carbonate, the acid |
| | | titrant is changed to sulfuric acid, and the pH is |
| | | maintained greater than pH 6.8 and less than pH 7.2. 80 |
| | | g of methyl myristate added. |
| 6 | h post depletion | End constant feed of glucose/methanol feed solution. |
| 6 | h post depletion | Aeration decreased to 0.5 slpm over 1 hour. |
| 6 | h post depletion | Agitation decreased to 1000 RPM over 3 h. |
| 6 | h post depletion | Modified pH-stat control feed initiated: |
| | | If pH > 7.0 then glucose/methanol feed solution |
| | | is delivered at a rate of 15 g glucose per liter of |
| | | initial volume per hour for a period of 15 min |
| | | and then halted. |
| | | If pH > 7.15 then delivery of the |
| | | glucose/methanol feed solution is ceased until |
| | | the pH decreases below 7.15. |
| | | If pH > 7.15 and specific oxygen uptake rate < 2.6 |
| | | mmol of oxygen/g dry cell weight/h then |
| | | glucose/methanol feed solution is delivered at |
| | | 15 g glucose per liter of initial volume per hour |
| | | until the pH decreases below 7.15. |
| 7 | h post depletion | Aeration now at 0.5 slpm. |
| 9 | h post depletion | Agitation now at 1000 RPM. |
| 27 | h post depletion | Agitation decreased to 800 RPM over 20 h. |
| 47 | h post depletion | Agitation now at 800 RPM. |

TABLE A2

| | Time | | | | | | | |
|---|---|---|---|---|---|---|---|---|
| | | Initial | | | | | | |
| | | batch | | | | | | |
| | Fermentation | glucose | | | | | | |
| | begins | depletion | 3 h | 6 h | 7 h | 9 h | 27 h | 47 h |
| Temp | 32° C. | 32° C. | Increase to | 35° C. | 35° C. | 35° C. | 35° C. | 35° C. |
| | | | 35° C. | | | | | |
| Agitation | 1200 | 1200 | 1200 | Ramp | Ramp | 1000 | Ramp | 800 |
| | RPM | RPM | RPM | down | down | RPM | down | RPM |
| Aeration | 1.5 slpm | 1.5 slpm | 1.5 slpm | Ramp | 0.5 | 0.5 | 0.5 | 0.5 |
| | | | | down | slpm | slpm | slpm | slpm |
| pH | ~6.8 | ~6.8 | Titrants | Maintain | Maintain | Maintain | Maintain | Maintain |
| | | | changed, | between | between | between | between | between |
| | | | maintain | 6.8 and | 6.8 and | 6.8 and | 6.8 and | 6.8 and |
| | | | between | 7.2 | 7.2 | 7.2 | 7.2 | 7.2 |
| | | | 6.8 and 7.2 | | | | | |
| Feed | none | Constant | Constant | End | pH- | pH- | pH- | pH- |
| | | 2 g/(l*h) | 2 g/(l*h) | constant, | stat | stat | stat | stat |
| | | | | Begin | control feed | control feed | control feed | control feed |
| | | | | pH-stat control | | | | |
| Other | | | 80 g methyl | | | | | |
| | | | myristate | | | | | |
| | | | added | | | | | |

### Example A

Recombinant Strain 1 was used to prepare deletion strains with each deletion strain comprising a deletion of a single thioesterase gene. Each deletion strain was prepared by deleting the single thioesterase gene with CRISPR-assisted genome-editing technology. CRISPR was used to delete the single thioesterase gene from the 5′ end starting 36 base pairs downstream of the start codon to the 3′ end at the stop codon (leaving the stop codon intact). Each deletion strain was then transformed with a Type 1 plasmid comprising an inducible C-8 specific Asch variant, Asch(T184I, F236L, V268A, V296A, V317A, S328G). A control strain was also prepared with Recombinant Strain 1 and the Type 1 plasmid comprising an inducible C-8 specific Asch variant, Asch(T184I, F236L, V268A, V296A, V317A, S328G). The deletion strains are described below in Table B.

TABLE B

| Deleted thioesterase gene | SEQ ID NO: |
|---|---|
| cheB | 32 |
| yigL | 33 |
| ybgC | 34 |
| yjjU | 1 |
| yfcE | 35 |
| yeiG | 36 |
| ybaW | 2 |
| frmB | 3 |
| yiiD | 37 |
| yqiA | 38 |
| ybfF | 39 |

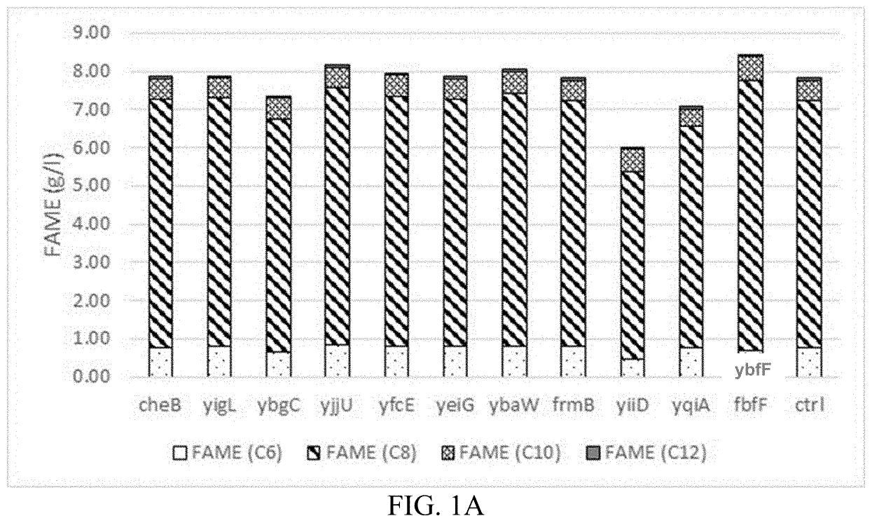

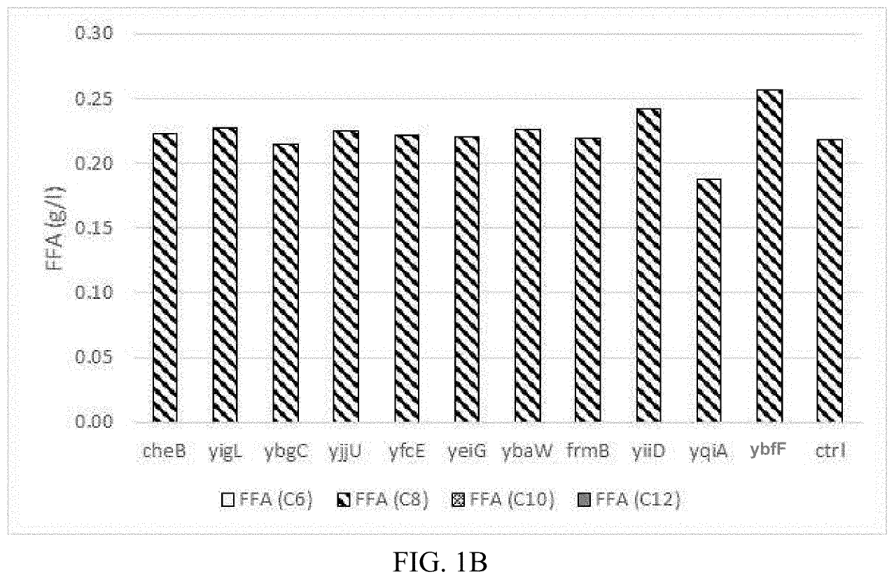

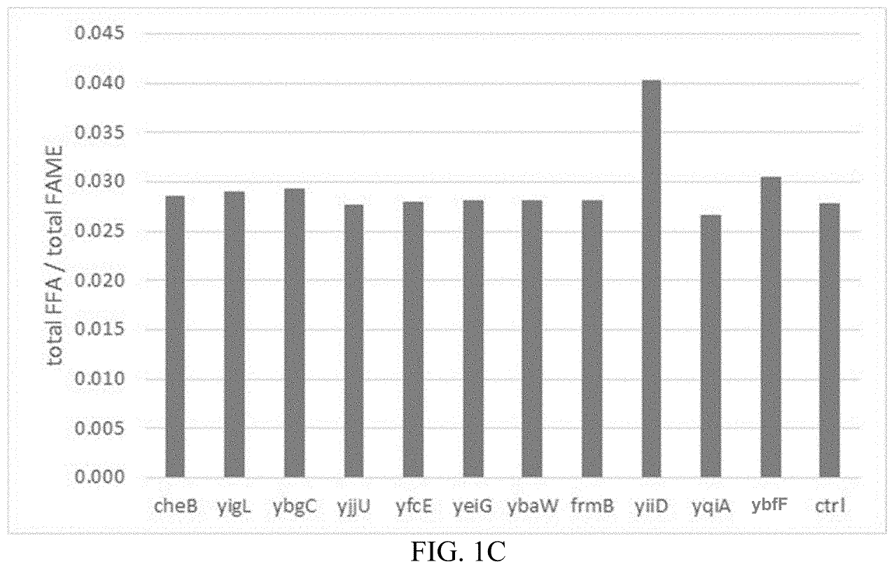

Each transformed deletion strain (including the control strain) was then grown as described above in the shake flask protocol and assayed for production of C6, C8, C10, C12 fatty acid methyl esters (FAMEs) and C6, C8, C10, and C12 free fatty acids (FFAs). The results for production of FAME is listed below in Table C and FIG. 1A. The results for production of FFA is listed below in Table D and FIG. 1B. The results for total FFA/total FAME is listed below in Table E and FIG. 1C.

TABLE C

| | Methyl | Methyl | Methyl | Methyl | total |
|---|---|---|---|---|---|
| | Hexanoate | Octanoate | Decanoate | Dodecanoate | FAME |
| deletion | (C6) g/L | (C8) g/L | (C10) g/L | (C12) g/L | g/l |
| cheB | 0.77 | 6.51 | 0.51 | 0.06 | 7.86 |
| yigL | 0.79 | 6.52 | 0.50 | 0.06 | 7.89 |
| ybgC | 0.67 | 6.09 | 0.54 | 0.06 | 7.37 |
| yjjU | 0.85 | 6.74 | 0.51 | 0.06 | 8.16 |
| yfcE | 0.81 | 6.55 | 0.53 | 0.06 | 7.97 |
| yeiG | 0.83 | 6.46 | 0.51 | 0.06 | 7.86 |
| ybaW | 0.82 | 6.62 | 0.54 | 0.06 | 8.04 |
| frmB | 0.79 | 6.45 | 0.53 | 0.06 | 7.83 |
| yiiD | 0.47 | 4.88 | 0.60 | 0.07 | 6.02 |
| yqiA | 0.79 | 5.77 | 0.45 | 0.07 | 7.07 |
| ybfF | 0.72 | 7.05 | 0.61 | 0.07 | 8.44 |
| control | 0.79 | 6.46 | 0.53 | 0.06 | 7.84 |

TABLE D

| | Hexanoic | Octanoic | Decanoic | Dodecanoic | total |
|---|---|---|---|---|---|
| | acid (C6) | acid (C8) | acid (C10) | acid (C12) | FFA |
| deletion | g/l | g/l | g/l | g/l | g/l |
| cheB | 0.00 | 0.22 | 0.00 | 0.00 | 0.22 |
| yigL | 0.00 | 0.23 | 0.00 | 0.00 | 0.23 |
| ybgC | 0.00 | 0.21 | 0.00 | 0.00 | 0.21 |
| yjjU | 0.00 | 0.23 | 0.00 | 0.00 | 0.23 |
| yfcE | 0.00 | 0.22 | 0.00 | 0.00 | 0.22 |
| yeiG | 0.00 | 0.22 | 0.00 | 0.00 | 0.22 |
| ybaW | 0.00 | 0.23 | 0.00 | 0.00 | 0.23 |
| frmB | 0.00 | 0.22 | 0.00 | 0.00 | 0.22 |
| yiiD | 0.00 | 0.24 | 0.00 | 0.00 | 0.24 |
| yqiA | 0.00 | 0.19 | 0.00 | 0.00 | 0.19 |
| ybfF | 0.00 | 0.26 | 0.00 | 0.00 | 0.26 |
| control | 0.00 | 0.22 | 0.00 | 0.00 | 0.22 |

TABLE E

| | | | total |
|---|---|---|---|
| | total | total | FFA/total |
| deletion | FAME g/l | FFA g/l | FAME |
| cheB | 7.86 | 0.22 | 0.028 |
| yigL | 7.89 | 0.23 | 0.029 |
| ybgC | 7.37 | 0.21 | 0.029 |
| yjjU | 8.16 | 0.23 | 0.028 |
| yfcE | 7.97 | 0.22 | 0.028 |
| yeiG | 7.86 | 0.22 | 0.028 |
| ybaW | 8.04 | 0.23 | 0.028 |
| frmB | 7.83 | 0.22 | 0.028 |
| yiiD | 6.02 | 0.24 | 0.040 |
| yqiA | 7.07 | 0.19 | 0.027 |
| ybfF | 8.44 | 0.26 | 0.030 |
| control | 7.84 | 0.22 | 0.028 |

As shown in Table C and FIG. 1A, the assayed strains with the C8-specific Asch variant and with corresponding deletions from Table B did not show substantially increased total FAME production compared to the control strain. As shown in Table D and FIG. 1B, the assayed strains with the C8-specific Asch variant and with corresponding deletions from Table B did not show substantially decreased total FFA production compared to the control strain. As shown in Table E and FIG. 1C, the assayed strains with the C8-specific Asch variant and with corresponding deletions from Table B did not show substantially improved total FFA/total FAME ratios.

### Example B

The deletion strains prepared in Example A and listed in Table B were then assayed with a C-10 specific Asch variant. Each deletion strain was transformed with a Type 1 plasmid comprising an inducible C-10 specific Asch variant, Asch(V296A). A control strain was also prepared with Recombinant Strain 1 and the Type 1 plasmid comprising an inducible C-10 specific Asch variant, Asch(V296A).

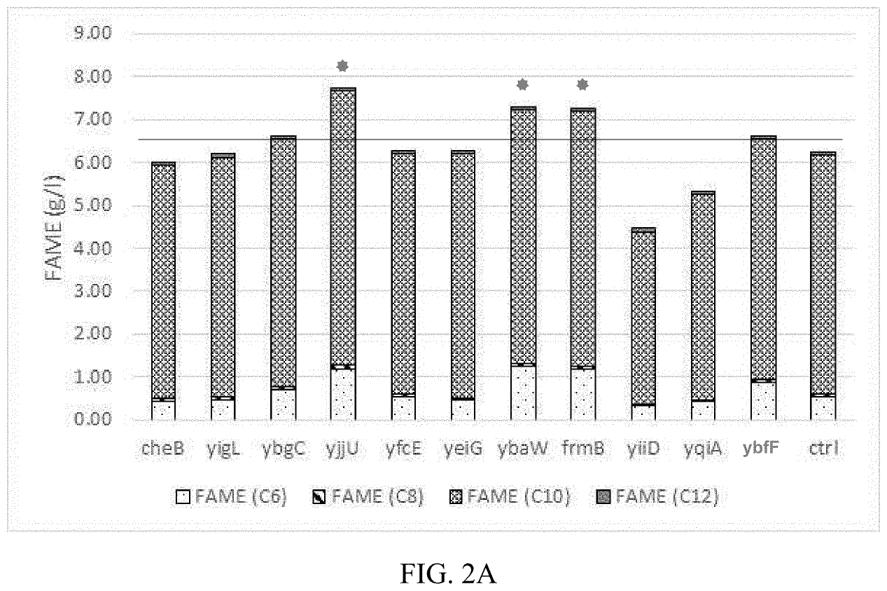

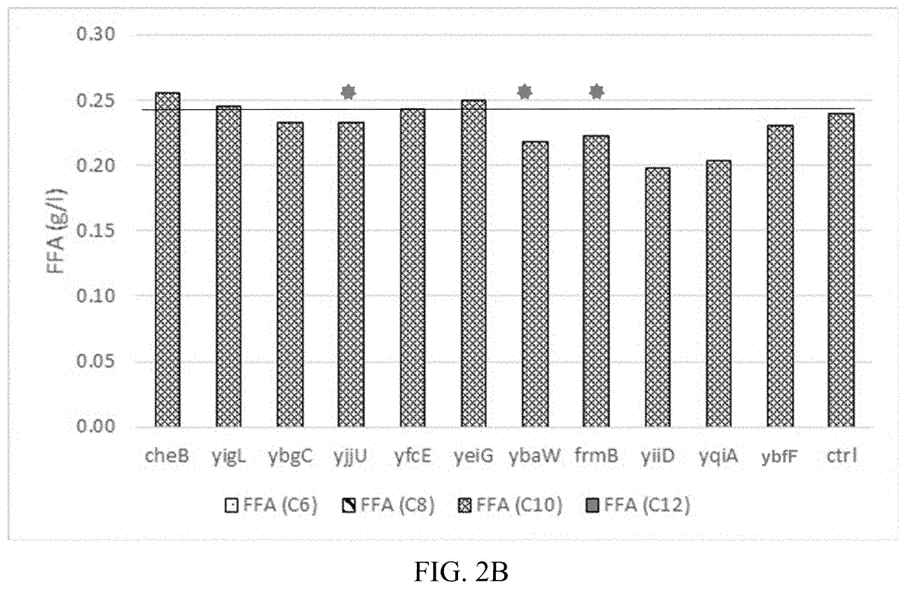

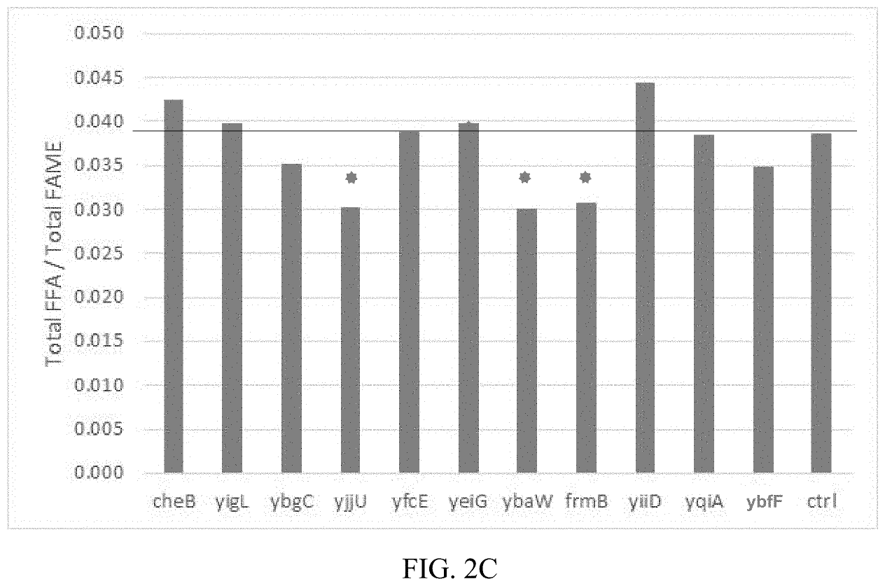

Each transformed deletion strain (including the control strain) was then grown as described above in the shake flask protocol and assayed for production of C6, C8, C10, C12 fatty acid methyl esters (FAMEs) and C6, C8, C10, and C12 free fatty acids (FFAs). The results for production of FAME is listed below in Table F and FIG. 2A. The results for production of FFA is listed below in Table G and FIG. 2B. The results for total FFA/total FAME is listed below in Table H and FIG. 2C.

TABLE F

| | Methyl | Methyl | Methyl | Methyl | total |
|---|---|---|---|---|---|
| | Hexanoate | Octanoate | Decanoate | Dodecanoate | FAME |
| deletion | (C6) g/L | (C8) g/L | (C10) g/L | (C12) g/L | g/l |
| cheB | 0.43 | 0.06 | 5.45 | 0.08 | 6.01 |
| yigL | 0.48 | 0.06 | 5.58 | 0.07 | 6.19 |
| ybgC | 0.72 | 0.05 | 5.78 | 0.07 | 6.63 |
| yjjU | 1.19 | 0.09 | 6.38 | 0.08 | 7.74 |
| yfcE | 0.55 | 0.06 | 5.59 | 0.08 | 6.27 |
| yeiG | 0.45 | 0.06 | 5.69 | 0.08 | 6.27 |
| ybaW | 1.24 | 0.07 | 5.90 | 0.08 | 7.29 |
| frmB | 1.19 | 0.07 | 5.94 | 0.08 | 7.27 |
| yiiD | 0.33 | 0.03 | 4.02 | 0.07 | 4.46 |
| yqiA | 0.42 | 0.05 | 4.77 | 0.07 | 5.32 |
| ybfF | 0.86 | 0.06 | 5.61 | 0.08 | 6.62 |
| ctrl | 0.54 | 0.06 | 5.56 | 0.08 | 6.24 |

TABLE G

| | Hexanoic | Octanoic | Decanoic | Dodecanoic | total |
|---|---|---|---|---|---|
| | acid (C6) | acid (C8) | acid (C10) | acid (C12) | FFA |
| deletion | g/l | g/l | g/l | g/l | g/l |
| cheB | 0.00 | 0.00 | 0.26 | 0.00 | 0.26 |
| yigL | 0.00 | 0.00 | 0.25 | 0.00 | 0.25 |
| ybgC | 0.00 | 0.00 | 0.23 | 0.00 | 0.23 |
| yjjU | 0.00 | 0.00 | 0.23 | 0.00 | 0.23 |
| yfcE | 0.00 | 0.00 | 0.24 | 0.00 | 0.24 |
| yeiG | 0.00 | 0.00 | 0.25 | 0.00 | 0.25 |
| ybaW | 0.00 | 0.00 | 0.22 | 0.00 | 0.22 |
| frmB | 0.00 | 0.00 | 0.22 | 0.00 | 0.22 |
| yiiD | 0.00 | 0.00 | 0.20 | 0.00 | 0.20 |
| yqiA | 0.00 | 0.00 | 0.20 | 0.00 | 0.20 |
| ybfF | 0.00 | 0.00 | 0.23 | 0.00 | 0.23 |
| ctrl | 0.00 | 0.00 | 0.24 | 0.00 | 0.24 |

TABLE H

| | | | total |
|---|---|---|---|
| | total | total | FFA/total |
| deletion | FAME g/l | FFA g/l | FAME |
| cheB | 6.01 | 0.26 | 0.042 |
| yigL | 6.19 | 0.25 | 0.040 |
| ybgC | 6.63 | 0.23 | 0.035 |
| yjjU | 7.74 | 0.23 | 0.030 |
| yfcE | 6.27 | 0.24 | 0.039 |
| yeiG | 6.27 | 0.25 | 0.040 |
| ybaW | 7.29 | 0.22 | 0.030 |
| frmB | 7.27 | 0.22 | 0.031 |
| yiiD | 4.46 | 0.20 | 0.044 |
| yqiA | 5.32 | 0.20 | 0.038 |
| ybfF | 6.62 | 0.23 | 0.035 |
| ctrl | 6.24 | 0.24 | 0.039 |

As shown in Table F and FIG. 2A, three of the assayed strains with the C10-specific Asch variant and with corresponding deletions of yjjU, ybaW, and frmB showed increased total FAME production compared to the control strain. As shown in Table G and FIG. 2B, the assayed strains with the C10-specific Asch variant and with corresponding deletions of yjjU, ybaW, and frmB showed decreased total FFA production compared to the control strain. As shown in Table H and FIG. 2C, three of the assayed strains with the C10-specific Asch variant and with corresponding deletions of yjjU, ybaW, and frmB showed improved total FFA/total FAME ratios. This example shows that the assayed strains corresponding to the yjjU, ybaW, and frmB deletions with the C10-specific Asch variant (V296A) showed increased FAME production and reduced FFA production in a C10 production background.

### Example C

Recombinant Strain 1 was used to prepare a ydiI deletion strain comprising a deletion of the ydiI gene (SEQ ID NO: 40). The ydiI deletion strain was prepared by deleting the ydiI gene with CRISPR-assisted genome-editing technology from the Recombinant Strain 1. CRISPR was used to delete the ydiI gene from the 5′ end starting 36 base pairs downstream of the start codon to the 3′ end at the stop codon (leaving the stop codon intact). The ydiI deletion strain was then transformed with a Type 1 plasmid comprising an inducible C8/C10 specific Asch variant, Asch(T184I, V268A, V296A) to make a ΔydiI-C8/C10 strain. The ydiI deletion strain was also transformed with a Type 1 plasmid comprising an inducible C10 specific Asch variant, Asch(V296A) to make a ΔydiI-C10 strain. Control strains were also prepared with Recombinant Strain 1 and the Type 1 plasmid comprising an inducible C8/C10 specific Asch variant, Asch(T184I, V268A, V296A) and Recombinant Strain 1 and the Type 1 plasmid comprising an inducible C10 specific Asch variant, Asch(V296A). The deletion strains are described below in Table I.

TABLE I

| | Additional | |
|---|---|---|
| Strain | deletion | Asch variant |
| ΔydiI-C8/C10 | ΔydiI | Asch(T184I, V268A, V296A) |
| ΔydiI-C10 | ΔydiI | Asch(V296A) |
| Control C8/C10 | — | Asch(T184I, V268A, V296A) |
| Control C10 | — | Asch(V296A) |

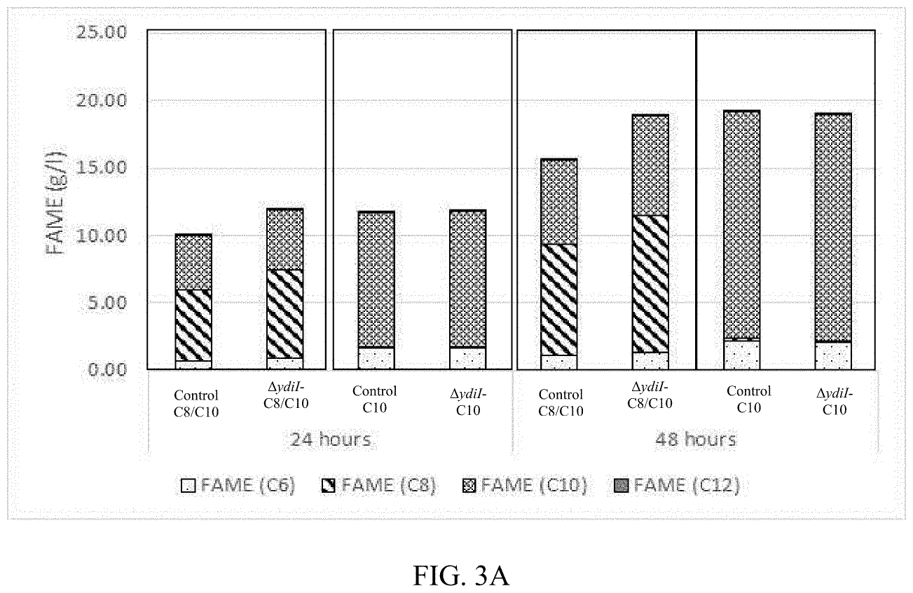

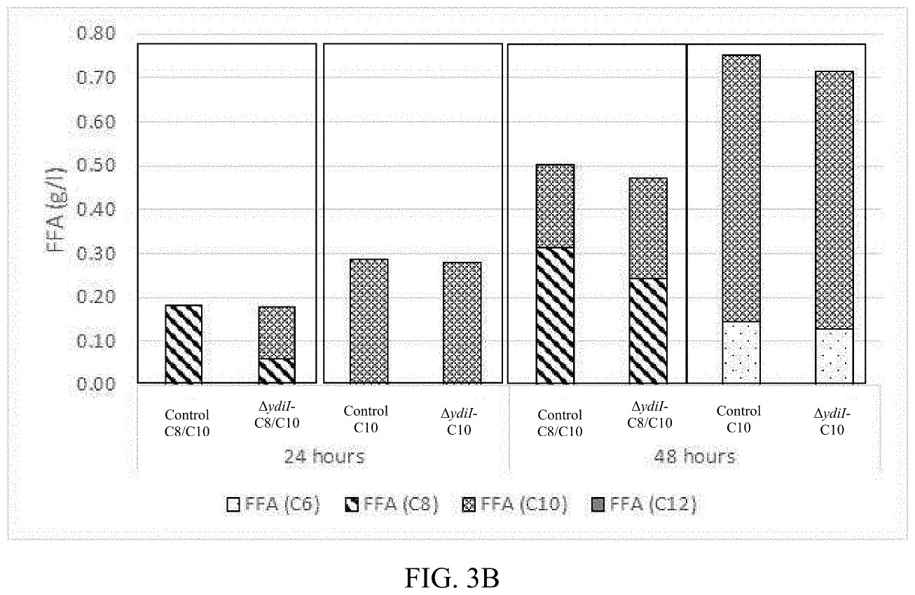

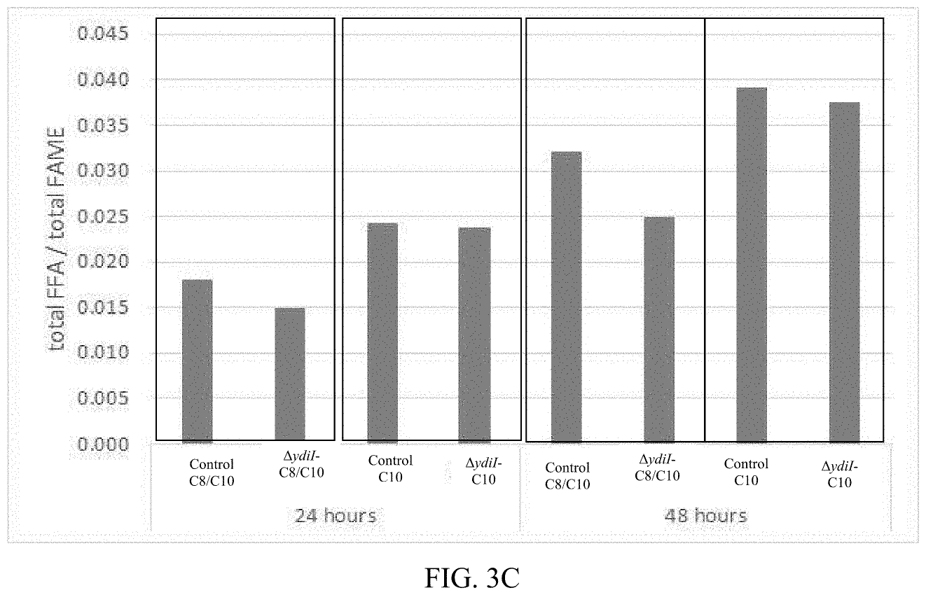

Each transformed deletion strain (including the control strains) was then grown as described above in the shake flask protocol and assayed at 24 h and 48 h for production of C6, C8, C10, C12 fatty acid methyl esters (FAMEs) and C6, C8, C10, and C12 free fatty acids (FFAs). The results for production of FAME is listed below in Table J and FIG. 3A. The results for production of FFA is listed below in Table K and FIG. 3B. The results for total FFA/total FAME is listed below in Table L and FIG. 3C.

TABLE J

| | | Methyl | Methyl | Methyl | Methyl | |
|---|---|---|---|---|---|---|
| Fermentation | | Hexanoate | Octanoate | Decanoate | Dodecanoate | total FAME |
| time | Strain | (C6) g/L | (C8) g/L | (C10) g/L | (C12) g/L | g/l |
| 24 h | Control | 0.75 | 5.28 | 3.94 | 0.09 | 10.06 |
| | C8/C10 | | | | | |
| | ΔydiI-C8/C10 | 0.98 | 6.45 | 4.43 | 0.09 | 11.95 |
| | Control C10 | 1.68 | 0.11 | 9.91 | 0.09 | 11.79 |
| | ΔydiI-C10 | 1.64 | 0.11 | 10.01 | 0.09 | 11.85 |
| 48 h | Control | 1.10 | 8.30 | 6.16 | 0.11 | 15.67 |
| | C8/C10 | | | | | |
| | ΔydiI-C8/C10 | 1.36 | 10.11 | 7.42 | 0.11 | 19.00 |
| | Control C10 | 2.23 | 0.14 | 16.79 | 0.11 | 19.27 |
| | ΔydiI-C10 | 2.08 | 0.14 | 16.75 | 0.11 | 19.07 |

TABLE K

| | | | | | Dodecanoic | |
|---|---|---|---|---|---|---|
| Fermentation | | Hexanoic acid | Octanoic acid | Decanoic acid | acid | |
| time | Strain | (C6) g/L | (C8) g/L | (C10) g/L | (C12) g/L | total FFA g/l |
| 24 h | Control | 0.00 | 0.18 | 0.00 | 0.00 | 0.18 |
| | C8/C10 | | | | | |
| | ΔydiI-C8/C10 | 0.00 | 0.06 | 0.12 | 0.00 | 0.18 |
| | Control C10 | 0.00 | 0.00 | 0.29 | 0.00 | 0.29 |
| | ΔydiI-C10 | 0.00 | 0.00 | 0.28 | 0.00 | 0.28 |
| 48 h | Control | 0.00 | 0.31 | 0.19 | 0.00 | 0.50 |
| | C8/C10 | | | | | |
| | ΔydiI-C8/C10 | 0.00 | 0.24 | 0.23 | 0.00 | 0.47 |
| | Control C10 | 0.15 | 0.00 | 0.61 | 0.00 | 0.75 |
| | ΔydiI-C10 | 0.13 | 0.00 | 0.59 | 0.00 | 0.71 |

TABLE L

| | | | | total |
|---|---|---|---|---|
| Fermentation | | Total | total | FFA/total |
| time | Strain | FAME g/l | FFA g/l | FAME |
| 24 hrs | Control C8/C10 | 10.06 | 0.18 | 0.018 |
| | ΔydiI-C8/C10 | 11.95 | 0.18 | 0.015 |
| | Control C10 | 11.79 | 0.29 | 0.024 |
| | ΔydiI-C10 | 11.85 | 0.28 | 0.024 |
| 48 hrs | Control C8/C10 | 15.67 | 0.50 | 0.032 |
| | ΔydiI-C8/C10 | 19.00 | 0.47 | 0.025 |
| | Control C10 | 19.27 | 0.75 | 0.039 |
| | Δydil-C10 | 19.07 | 0.71 | 0.037 |

As shown in Table J and FIG. 3A, the ΔydiI-C8/C10 strain showed increased total FAME production compared to the C8/C10 control strain at 24 h and 48 h. The ΔydiI-C10 strain showed similar total FAME production compared to the C10 control strain at 24 h and 48 h.

As shown in Table K and FIG. 3B, the ΔydiI-C8/C10 strain showed slightly reduced total FFA production compared to the C8/C10 control strain at 24 h and 48 h. The ΔydiI-C10 strain showed slightly reduced total FFA production compared to the C10 control strain at 24 h and 48 h. The ΔydiI-C8/C10 strain showed reduced C8 FFA production compared to the C8/C10 control strain at 24 h and 48 h.

As shown in Table L and FIG. 3C, the ΔydiI-C8/C10 strain showed improved total FFA/total FAME ratios at 24 h and 48 h. The ΔydiI-C10 strain showed similar total FFA/total FAME ratios at 24 h and slightly improved total FFA/total FAME ratios at 48 h.

This example shows that the ΔydiI-C8/C10 strain showed increased total FAME production, reduced FFA production, and improved total FFA/total FAME ratios. This example shows that the ΔydiI-C8/C10 strain showed reduced formation of C8 FFA.

### Example D

Recombinant Strain 1 was used to prepare a ydiI deletion strain comprising a deletion of the ydiI gene (SEQ ID NO: 40). The ydiI deletion strain was prepared by deleting the ydiI gene with CRISPR-assisted genome-editing technology from the Recombinant Strain 1. CRISPR was used to delete the ydiI gene from the 5′ end starting 36 base pairs downstream of the start codon to the 3′ end at the stop codon (leaving the stop codon intact). The ydiI deletion strain was then transformed with a Type 1 plasmid comprising an inducible C10 specific Asch variant, Asch(V296A) to make a ΔydiI-C10 strain.

Recombinant Strain 1 was used to prepare a bioH deletion strain comprising a deletion of the bioH gene (SEQ ID NO: 41). The bioH deletion strain was prepared by deleting the bioH gene with CRISPR-assisted genome-editing technology from the Recombinant Strain 1. CRISPR was used to delete the bioH gene from the 5′ end starting 36 base pairs downstream of the start codon to the 3′ end at the stop codon (leaving the stop codon intact). The bioH deletion strain was then transformed with a Type 1 plasmid comprising an inducible C10 specific Asch variant, Asch(V296A) to make a ΔbioH-C10 strain. Recombinant Strain 1 was used to prepare a ydiI-bioH deletion strain comprising a deletion of the ydiI gene (SEQ ID NO: 40) and the bioH gene (SEQ ID NO: 41) as described above. The ydiI-bioH deletion strain was then transformed with a Type 1 plasmid comprising an inducible C10 specific Asch variant, Asch(V296A) to make a ΔydiI-ΔbioH-C10 strain. A control strain was also prepared with Recombinant Strain 1 and the Type 1 plasmid comprising an inducible C10 specific Asch variant, Asch(V296A). The deletion strains are described below in Table M.

TABLE M

| | Additional | |
|---|---|---|
| Strain | deletion | Asch variant |
| ΔydiI-C10 | ΔydiI | Asch(V296A) |
| ΔbioH-C10 | ΔbioH | Asch(V296A) |
| ΔydiI-ΔbioH-C10 | ΔydiI, ΔbioH | Asch(V296A) |
| Control C10 | — | Asch(V296A) |

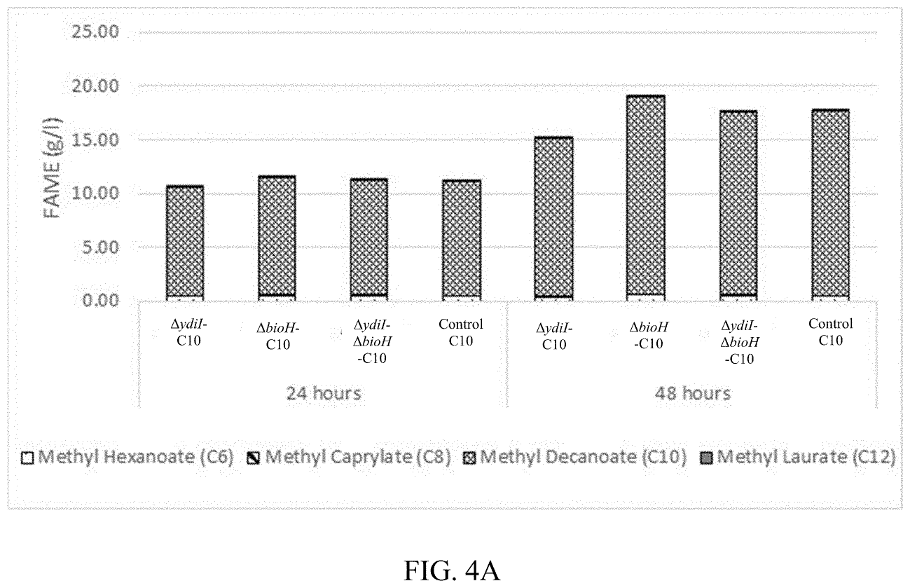

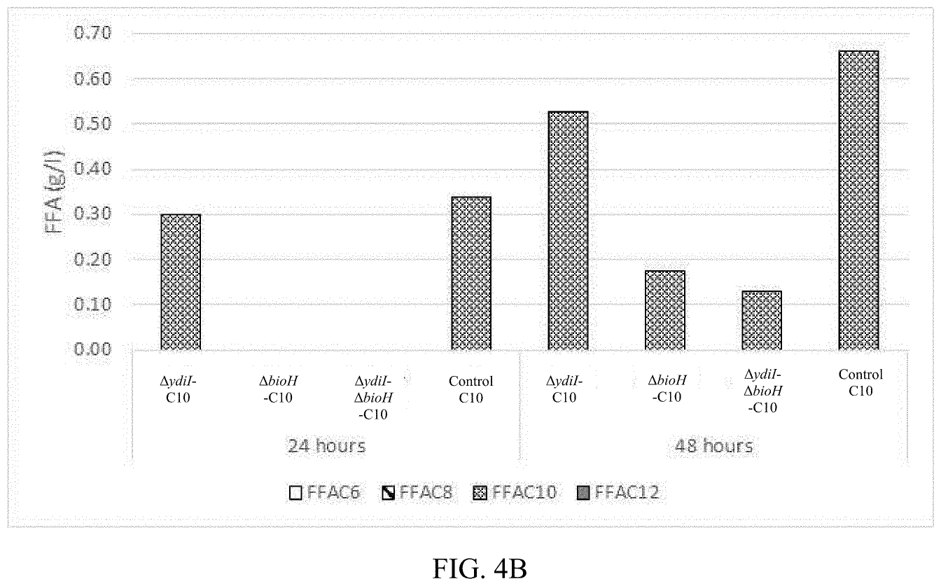

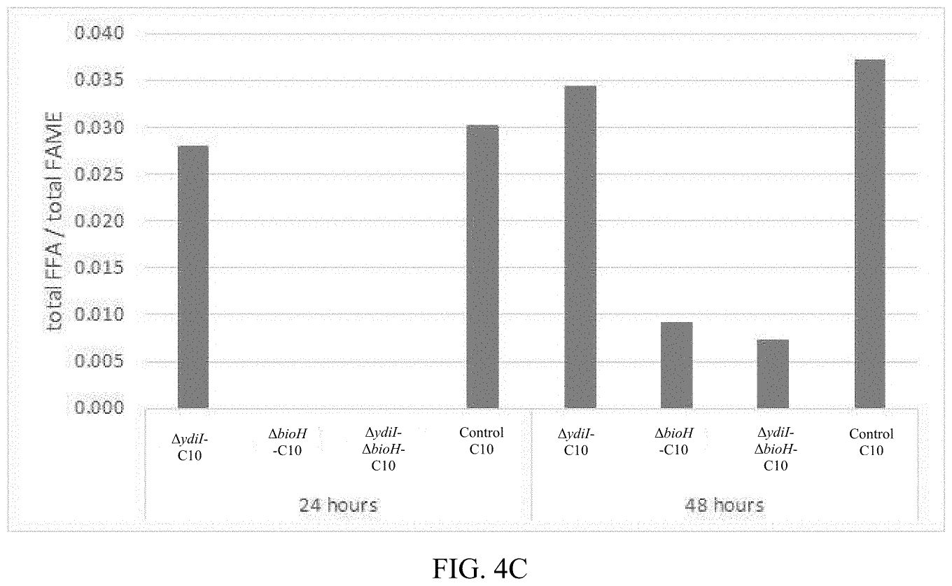

Each transformed deletion strain (including the control strains) was then grown as described above in the shake flask protocol and assayed at 24 h and 48 h for production of C6, C8, C10, C12 fatty acid methyl esters (FAMEs) and C6, C8, C10, and C12 free fatty acids (FFAs). The results for production of FAME is listed below in Table N and FIG. 4A. The results for production of FFA is listed below in Table O and FIG. 4B. The results for total FFA/total FAME is listed below in Table P and FIG. 4C.

TABLE N

| | | Methyl | Methyl | Methyl | Methyl | |
|---|---|---|---|---|---|---|
| Fermentation | | Hexanoate | Octanoate | Decanoate | Dodecanoate | total FAME |
| time | Strain | (C6) g/L | (C8) g/L | (C10) g/L | (C12) g/L | g/l |
| 24 h | ΔydiI-C10 | 0.45 | 0.04 | 10.14 | 0.08 | 10.72 |
| | ΔbioH-C10 | 0.56 | 0.05 | 10.94 | 0.08 | 11.63 |
| | ΔydiI-ΔbioH- | 0.54 | 0.05 | 10.71 | 0.08 | 11.38 |
| | C10 | | | | | |
| | Control C10 | 0.48 | 0.04 | 10.58 | 0.09 | 11.19 |
| 48 h | ΔydiI-C10 | 0.44 | 0.05 | 14.70 | 0.10 | 15.28 |
| | ΔbioH-C10 | 0.58 | 0.05 | 18.38 | 0.09 | 19.11 |
| | ΔydiI-ΔbioH- | 0.54 | 0.05 | 17.06 | 0.10 | 17.75 |
| | C10 | | | | | |
| | Control C10 | 0.47 | 0.05 | 17.21 | 0.10 | 17.83 |

TABLE O

| | | | | | Dodecanoic | |
|---|---|---|---|---|---|---|
| Fermentation | | Hexanoic acid | Octanoic acid | Decanoic acid | acid | |
| time | Strain | (C6) g/L | (C8) g/L | (C10) g/L | (C12) g/L | total FFA g/l |
| 24 h | ΔydiI-C10 | 0.00 | 0.00 | 0.30 | 0.00 | 0.30 |
| | ΔbioH-C10 | 0.00 | 0.00 | 0.00 | 0.00 | 0.00 |
| | ΔydiI-ΔbioH- | 0.00 | 0.00 | 0.00 | 0.00 | 0.00 |
| | C10 | | | | | |
| | Control C10 | 0.00 | 0.00 | 0.34 | 0.00 | 0.34 |
| 48 h | ΔydiI-C10 | 0.00 | 0.00 | 0.53 | 0.00 | 0.53 |
| | ΔbioH-C10 | 0.00 | 0.00 | 0.18 | 0.00 | 0.18 |
| | ΔydiI-ΔbioH- | | | | | |
| | C10 | 0.00 | 0.00 | 0.13 | 0.00 | 0.13 |
| | Control C10 | 0.00 | 0.00 | 0.66 | 0.00 | 0.66 |

TABLE P

| | | total | total | total |
|---|---|---|---|---|
| Fermentation | | FAME | FFA | FFA/total |
| time | Strain | g/l | g/l | FAME |
| 24 h | ΔydiI-C10 | 10.72 | 0.30 | 0.028 |
| | ΔbioH-C10 | 11.63 | 0.00 | 0.000 |
| | ΔydiI-ΔbioH-C10 | 11.38 | 0.00 | 0.000 |
| | Control C10 | 11.19 | 0.34 | 0.030 |
| 48 h | ΔydiI-C01 | 15.28 | 0.53 | 0.034 |
| | ΔbioH-C10 | 19.11 | 0.18 | 0.009 |
| | ΔydiI-ΔbioH-C10 | 17.75 | 0.13 | 0.007 |
| | Control C10 | 17.83 | 0.66 | 0.037 |

As shown in Table N and FIG. 4A, the ΔydiI-C10 strain, the ΔbioH-C10 strain, and the ΔydiI-ΔbioH-C10 strain showed similar total FAME production compared to the C10 control strain at 24 h. The ΔydiI-C10 strain showed slightly decreased total FAME production compared to the C10 control strain at 48 h. The ΔbioH-C10 strain showed slightly increased total FAME production compared to the C10 control strain at 48 h. The ΔydiI-ΔbioH-C10 strain showed similar total FAME production compared to the C10 control strain at 48 h.

As shown in Table O and FIG. 4B, the ΔydiI-C10 strain showed slightly decreased total FFA production compared to the C10 control strain at 24 h and 48 h. The ΔbioH-C10 strain showed substantially decreased total FFA production compared to the C10 control strain at 24 h and 48 h. The ΔydiI-ΔbioH-C10 strain showed substantially decreased total FFA production compared to the C10 control strain at 24 h and 48 h.

As shown in Table P and FIG. 4C, the ΔydiI-C10 strain showed slightly improved total FFA/total FAME ratios compared to the C10 control strain at 24 h and 48 h. The ΔbioH-C10 strain showed substantially improved total FFA/total FAME ratios compared to the C10 control strain at 24 h and 48 h. The ΔydiI-ΔbioH-C10 strain showed substantially improved total FFA/total FAME ratios compared to the C10 control strain at 24 h and 48 h.

This example shows that the ΔbioH-C10 and ΔydiI-ΔbioH-C10 strains showed substantially reduced FFA production and substantially improved total FFA/total FAME ratios.

### Example E

Recombinant Strain 1 was used to prepare a ydiI deletion strain comprising a deletion of the ydiI gene (SEQ ID NO: 40). The ydiI deletion strain was prepared by deleting the ydiI gene with CRISPR-assisted genome-editing technology from the Recombinant Strain 1. CRISPR was used to delete the ydiI gene from the 5′ end starting 36 base pairs downstream of the start codon to the 3′ end at the stop codon (leaving the stop codon intact). The ydiI deletion strain was then transformed with a Type 1 plasmid comprising an inducible C8/C10 specific Asch variant, Asch(T184I, V268A, V296A) to make a ΔydiI-C8/C10 strain.

Recombinant Strain 1 was used to prepare a bioH deletion strain comprising a deletion of the bioH gene (SEQ ID NO: 41). The bioH deletion strain was prepared by deleting the bioH gene with CRISPR-assisted genome-editing technology from the Recombinant Strain 1. CRISPR was used to delete the bioH gene from the 5′ end starting 36 base pairs downstream of the start codon to the 3′ end at the stop codon (leaving the stop codon intact). The bioH deletion strain was then transformed with a Type 1 plasmid comprising an inducible C8/C10 specific Asch variant, Asch(T184I, V268A, V296A) to make a ΔbioH-C8/C10 strain. Recombinant Strain 1 was used to prepare a ydiI-bioH deletion strain comprising a deletion of the ydiI gene (SEQ ID NO: 40) and the bioH gene (SEQ ID NO: 41) as described above. The ydiI-bioH deletion strain was then transformed with a Type 1 plasmid comprising an inducible C8/C10 specific Asch variant, Asch(T184I, V268A, V296A) to make a ΔydiI-ΔbioH-C8/C10 strain. A control strain was also prepared with Recombinant Strain 1 and the Type 1 plasmid comprising an inducible C8/C10 specific Asch variant, Asch(T184I, V268A, V296A). The deletion strains are described below in Table Q.

TABLE Q

| Strain | Additional deletion | Asch variant |
|---|---|---|
| ΔydiI- C8/C10 | ΔydiI | Asch(T184I, V268A, V296A) |
| ΔbioH- C8/C10 | ΔbioH | Asch(T184I, V268A, V296A) |
| ΔydiI-ΔbioH- | ΔydiI, ΔbioH | Asch(T184I, V268A, V296A) |
| C8/C10 | | |
| Control C8/C10 | — | Asch(T184I, V268A, V296A) |

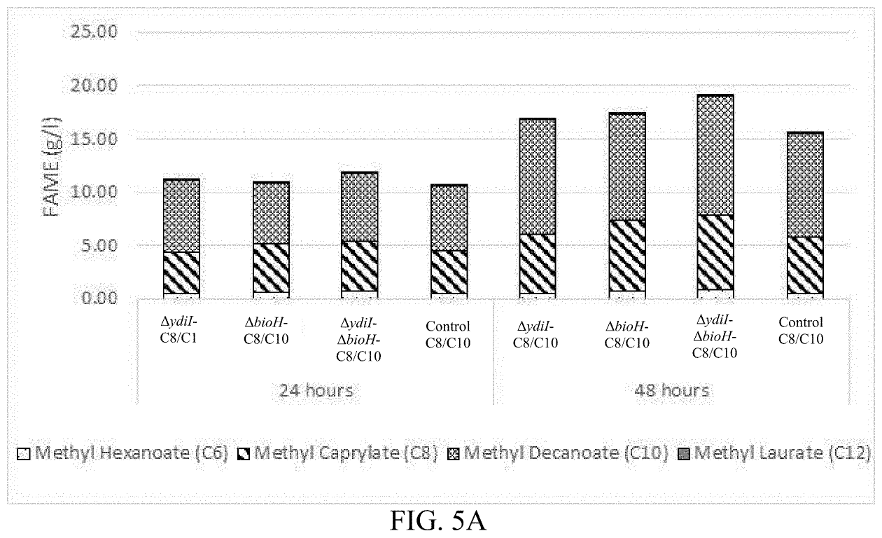

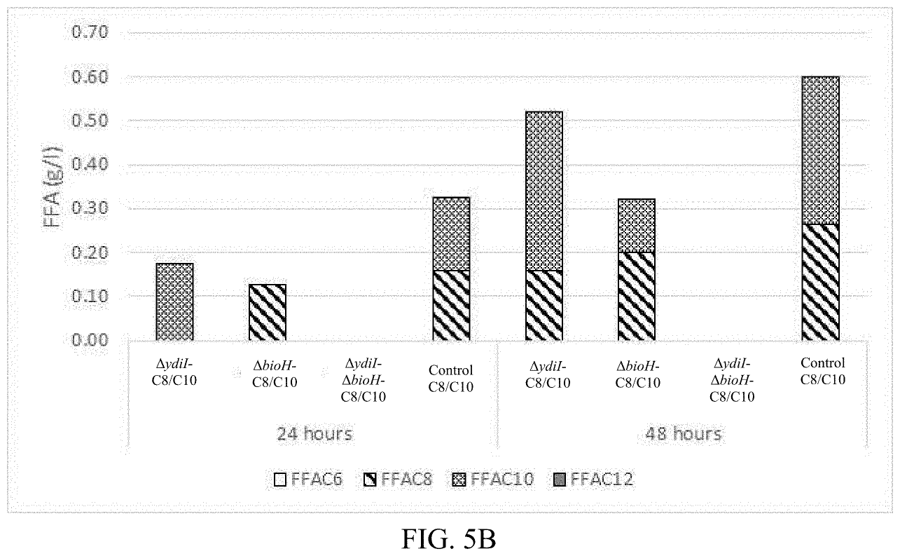

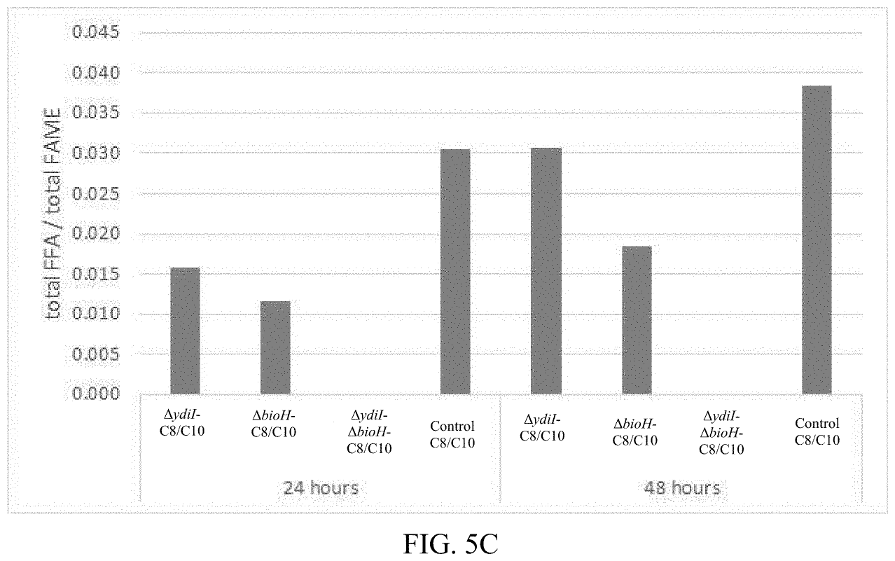

Each transformed deletion strain (including the control strains) was then grown as described above in the shake flask protocol and assayed at 24 h and 48 h for production of C6, C8, C10, C12 fatty acid methyl esters (FAMEs) and C6, C8, C10, and C12 free fatty acids (FFAs). The results for production of FAME is listed below in Table R and FIG. 5A. The results for production of FFA is listed below in Table S and FIG. 5B. The results for total FFA/total FAME is listed below in Table T and FIG. 5C.

TABLE R

| | | Methyl | Methyl | Methyl | Methyl | |
|---|---|---|---|---|---|---|
| Fermentation | | Hexanoate | Octanoate | Decanoate | Dodecanoate | total FAME |
| time | Strain | (C6) g/L | (C8) g/L | (C10) g/L | (C12) g/L | g/l |
| 24 h | ΔydiI-C8/C10 | 0.45 | 3.96 | 6.71 | 0.10 | 11.23 |
| | ΔbioH-C8/C10 | 0.59 | 4.58 | 5.75 | 0.09 | 11.01 |
| | ΔydiI-ΔbioH- | 0.71 | 4.67 | 6.36 | 0.10 | 11.84 |
| | C8/C10 | | | | | |
| | Control C8/C10 | 0.48 | 3.99 | 6.16 | 0.10 | 10.72 |
| 48 h | ΔydiI-C8/C10 | 0.54 | 5.53 | 10.80 | 0.13 | 17.00 |
| | ΔbioH-C8/C10 | 0.74 | 6.60 | 10.00 | 0.12 | 17.46 |
| | ΔydiI-ΔbioH- | 0.86 | 6.98 | 11.21 | 0.13 | 19.19 |
| | C8/C10 | | | | | |
| | Control C8/C10 | 0.54 | 5.31 | 9.70 | 0.12 | 15.68 |

TABLE S

| | | | | | Dodecanoic | |
|---|---|---|---|---|---|---|
| Fermentation | | Hexanoic acid | Octanoic acid | Decanoic acid | acid | |
| time | Strain | (C6) g/L | (C8) g/L | (C10) g/L | (C12) g/L | total FFA g/l |
| 24 h | ΔydiI-C8/C10 | 0.00 | 0.00 | 0.18 | 0.00 | 0.18 |
| | ΔbioH-C8/C10 | 0.00 | 0.13 | 0.00 | 0.00 | 0.13 |
| | ΔydiI-ΔbioH- | 0.00 | 0.00 | 0.00 | 0.00 | 0.00 |
| | C8/C10 | | | | | |
| | Control C8/C10 | 0.00 | 0.16 | 0.17 | 0.00 | 0.33 |
| 48 h | ΔydiI-C8/C10 | 0.00 | 0.16 | 0.36 | 0.00 | 0.52 |
| | ΔbioH-C8/C10 | 0.00 | 0.20 | 0.12 | 0.00 | 0.32 |
| | ΔydiI-ΔbioH- | 0.00 | 0.00 | 0.00 | 0.00 | 0.00 |
| | C8/C10 | | | | | |
| | Control C8/C10 | 0.00 | 0.26 | 0.34 | 0.00 | 0.60 |

TABLE T

| | | total | total | total |
|---|---|---|---|---|
| Fermentation | | FAME | FFA | FFA/total |
| time | Strain | g/l | g/l | FAME |
| 24 h | ΔydiI- C8/C10 | 11.23 | 0.18 | 0.016 |
| | ΔbioH- C8/C10 | 11.01 | 0.13 | 0.012 |
| | ΔydiI-ΔbioH-C8/C10 | 11.84 | 0.00 | 0.000 |
| | Control C8/C10 | 10.72 | 0.33 | 0.030 |
| 48 h | ΔydiI-C8/C10 | 17.00 | 0.52 | 0.031 |
| | ΔbioH-C8/C10 | 17.46 | 0.32 | 0.018 |
| | ΔydiI-ΔbioH-C8/C10 | 19.19 | 0.00 | 0.000 |
| | Control C8/C10 | 15.68 | 0.60 | 0.038 |

As shown in Table R and FIG. 5A, the ΔydiI-C8/C10 strain, the ΔbioH-C8/C10 strain, and the ΔydiI-ΔbioH-C8/C10 strain showed slightly increased total FAME production compared to the C8/C10 control strain at 24 h. The ΔydiI-C8/C10 strain, the ΔbioH-C8/C10 strain, and the ΔydiI-ΔbioH-C8/C10 strain showed increased total FAME production compared to the C8/C10 control strain at 48 h.

As shown in Table S and FIG. 5B, the ΔydiI-C8/C10 strain showed substantially decreased total FFA production compared to the C8/C10 control strain at 24 h. In particular, the ΔydiI-C8/C10 strain showed substantially decreased C8 FFA production compared to the C8/C10 control strain at 24 h. The ΔbioH-C8/10 strain showed substantially decreased total FFA production compared to the C8/10 control strain at 24 h. In particular, the ΔbioH-C8/C10 strain showed substantially decreased C10 FFA production compared to the C8/10 control strain at 24 h. The ΔydiI-ΔbioH-C8/C10 strain showed substantially decreased total FFA production compared to the C8/C10 control strain at 24 h, with the total FFA undetectable at the scale of the experiment. The ΔydiI-C8/C10 strain and the ΔbioH-C8/C10 strain both showed decreased total FFA production compared to the C8/C10 control strain at 48 h. The ΔydiI-ΔbioH-C8/C10 strain showed substantially decreased total FFA production compared to the C8/C10 control strain, decreased total FFA production more than the combination of each of that observed in the ΔydiI-C8/C10 strain and the ΔbioH-C8/C10 strain. This example also shows that the ΔydiI-C8/C10 strain reduces C8 FFA and that the ΔbioH-C8/C10 strain reduces C10 FFA.

As shown in Table T and FIG. 5C, the ΔydiI-C8/C10 strain showed substantially improved total FFA/total FAME ratios compared to the C8/C10 control strain at 24 h and 48 h. The ΔbioH-C8/C10 strain showed substantially improved total FFA/total FAME ratios compared to the C8/C10 control strain at 24 h and 48 h. The ΔydiI-ΔbioH-C8/C10 strain showed substantially improved total FFA/total FAME ratios compared to the C8/C10 control strain at 24 h and 48 h, with the total FFA undetectable at the scale of the experiment.

### Example F

Recombinant Strain 1 was used to prepare a ydiI-bioH deletion strain comprising a deletion of the ydiI gene (SEQ ID NO: 40) and the bioH gene (SEQ ID NO: 41) as described above. The ydiI-bioH deletion strain was then transformed with a Type 1 plasmid comprising an inducible C8/C10 specific Asch variant, Asch(T184I, V268A, V296A) to make a ΔydiI-ΔbioH-C8/C10 strain. A control strain was also prepared with Recombinant Strain 1 and the Type 1 plasmid comprising an inducible C8/C10 specific Asch variant, Asch(T184I, V268A, V296A).

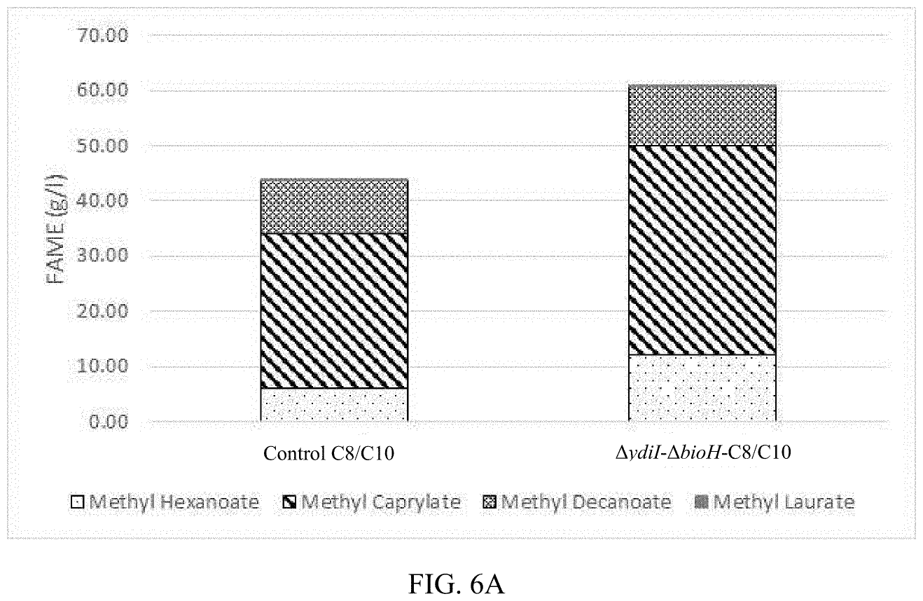

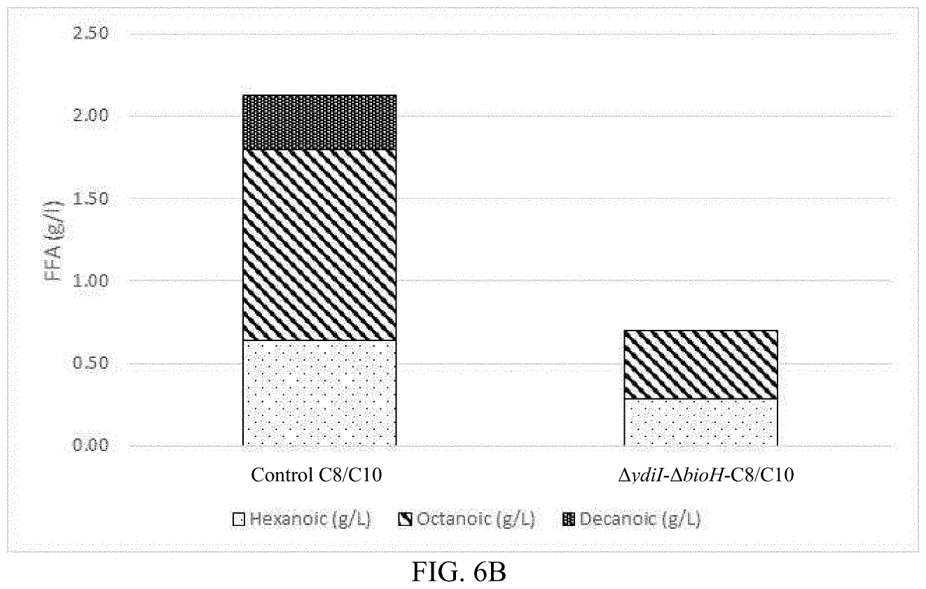

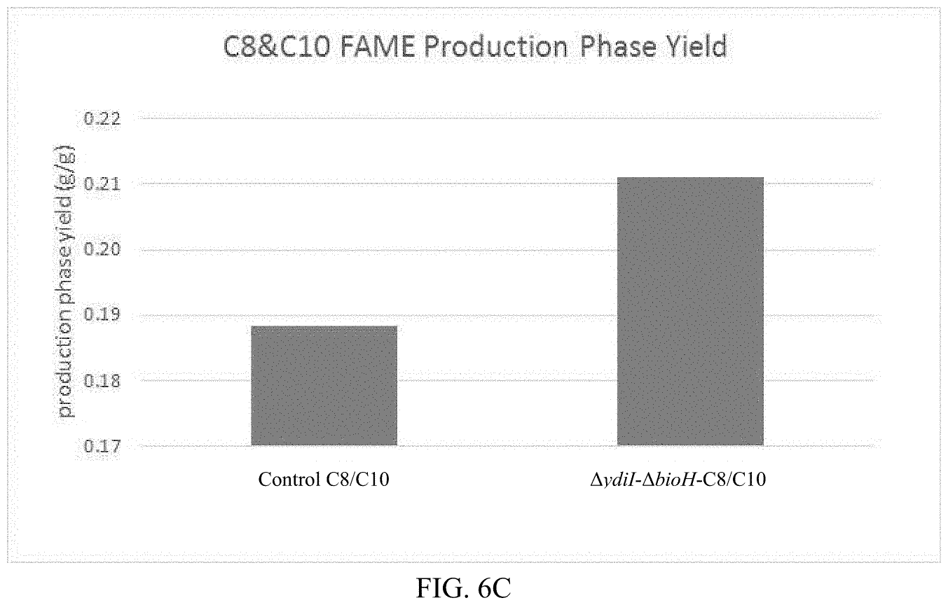

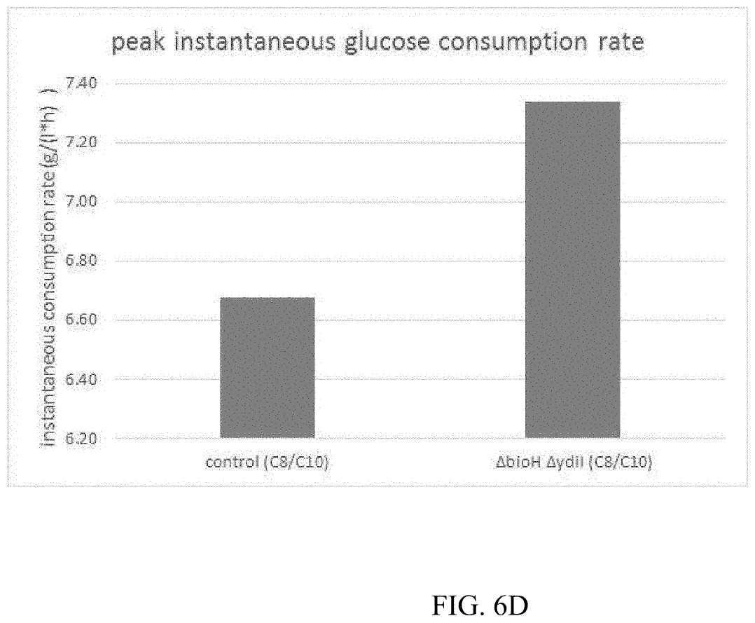

The ΔydiI-ΔbioH-C8/C10 and C8/C10 control strains were grown as described above in the fermenter protocol and assayed for production of C6, C8, C10, C12 fatty acid methyl esters (FAMEs) and C6, C8, C10, and C12 free fatty acids (FFAs). The results for production of FAME are listed below in Table U and FIG. 6A. The results for production of FFA are listed below in Table V and FIG. 6B. The C8 & C10 FAME production phase yield and peak instantaneous glucose consumption rate were also measured. The results for C8 & C10 FAME production phase yield and peak instantaneous glucose consumption rate are listed below in Table W. The results for C8 & C10 FAME production phase yield are shown in FIG. 6C. The results for peak instantaneous glucose consumption rate are shown in FIG. 6D.

TABLE U

| | Methyl | Methyl | Methyl | total |
|---|---|---|---|---|
| | Hexanoate | Octanoate | Decanoate | FAME |
| Strain | (C6) g/L | (C8) g/L | (C10) g/L | g/l |
| Control | 6.14 | 27.93 | 9.54 | 43.61 |
| C8/C10 | | | | |
| ΔydiI-ΔbioH-C8/C10 | 12.01 | 38.08 | 10.64 | 60.73 |

TABLE V

| | Hexanoic | Octanoic | Decanoic | |
|---|---|---|---|---|
| | acid (C6) | acid (C8) | acid (C10) | total |
| Strain | g/l | g/l | g/l | FFA g/l |
| Control | 0.64 | 1.16 | 0.32 | 2.12 |
| C8/C10 | | | | |
| ΔydiI-ΔbioH-C8/C10 | 0.29 | 0.41 | 0.00 | 0.70 |

TABLE W

| | C8 & C10 FAME | Peak instantaneous |
|---|---|---|
| | production | glucose consumption |
| Strain | phase yield (g/g) | rate (g/(l*h)) |
| Control C8/C10 | 0.19 | 6.68 |
| ΔydiI-ΔbioH-C8/C10 | 0.21 | 7.34 |

As shown in Table U and FIG. 6A, the ΔydiI-ΔbioH-C8/C10 strain showed substantially increased total FAME production compared to the C8/C10 control strain when grown according to the fermenter protocol.

As shown in Table V and FIG. 6B, the ΔydiI-ΔbioH-C8/C10 strain showed substantially decreased total FFA production compared to the C8/C10 control strain when grown according to the fermenter protocol. In particular, the ΔydiI-ΔbioH-C8/C10 strain showed no production of C10 FFA that was detectable at the scale of the experiment. The ΔydiI-ΔbioH-C8/C10 strain also showed substantially decreased production of C6 FFA and C8 FFA.

As shown in Table W and FIG. 6C, the ΔydiI-ΔbioH-C8/C10 strain showed improved C8 & C10 FAME production phase yield compared to the C8/C10 control strain. As shown in Table W and FIG. 6D, the ΔydiI-ΔbioH-C8/C10 strain showed improved peak instantaneous glucose consumption rate compared to the C8/C10 control strain. This example shows that the ΔydiI-ΔbioH-C8/C10 strain shows improved total FAME production, reduced production of FFA, improved C8 & C10 FAME production phase yield, and improved peak instantaneous glucose consumption rate.

### Example G

Recombinant Strain 1 was used to prepare a ydiI-bioH deletion strain as described above. The ydiI-bioH deletion strain was then further modified by deleting a tesA gene (SEQ ID NO: 42) to prepare a ydiI-bioH-tesA deletion strain. The ydiI-bioH deletion strain was then transformed with one of three Type 1 plasmids. The Type 1 plasmids comprised either an inducible C8 specific Asch, Asch(T184I, F236L, V268A, V296A, V317A, S328G), an inducible C8/C10 specific Asch variant, Asch(T184I, V268A, V296A), or an inducible C10 specific Asch variant, Asch(V296A). The ydiI-bioH-tesA deletion strain was then also transformed with one of the C8 specific Asch, C8/C10 specific Asch, or C10 specific Asch. The deletion strains are described below in Table X.

TABLE X

| Strain | Additional deletion | Asch variant |
|---|---|---|
| ΔydiI-ΔbioH-C8 | ΔydiI, ΔbioH | Asch(T184I, F236L, V268A, V296A, |
| | | V317A, S328G) |
| ΔydiI-ΔbioH- C8/C10 | ΔydiI, ΔbioH | Asch(T184I, V268A, V296A) |
| ΔydiI-ΔbioH-C10 | ΔydiI, ΔbioH | Asch(V296A) |
| ΔydiI-ΔbioH-ΔtesA-C8 | ΔydiI, ΔbioH, ΔtesA | Asch(T184I, F236L, V268A, V296A, |
| | | V317A, S328G) |
| ΔydiI-ΔbioH-ΔtesA C8/C10 | ΔydiI, ΔbioH, ΔtesA | Asch(T184I, V268A, V296A) |
| ΔydiI-ΔbioH-ΔtesA C10 | ΔydiI, ΔbioH, ΔtesA | Asch(V296A) |

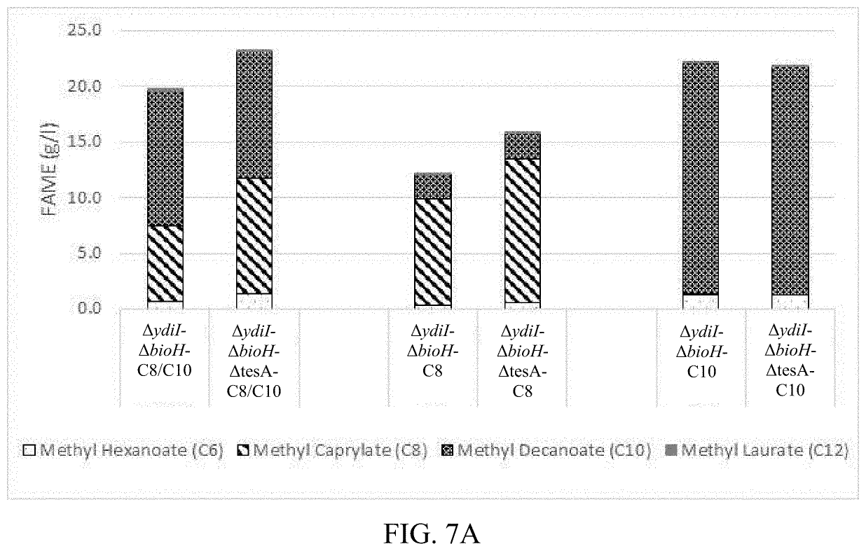

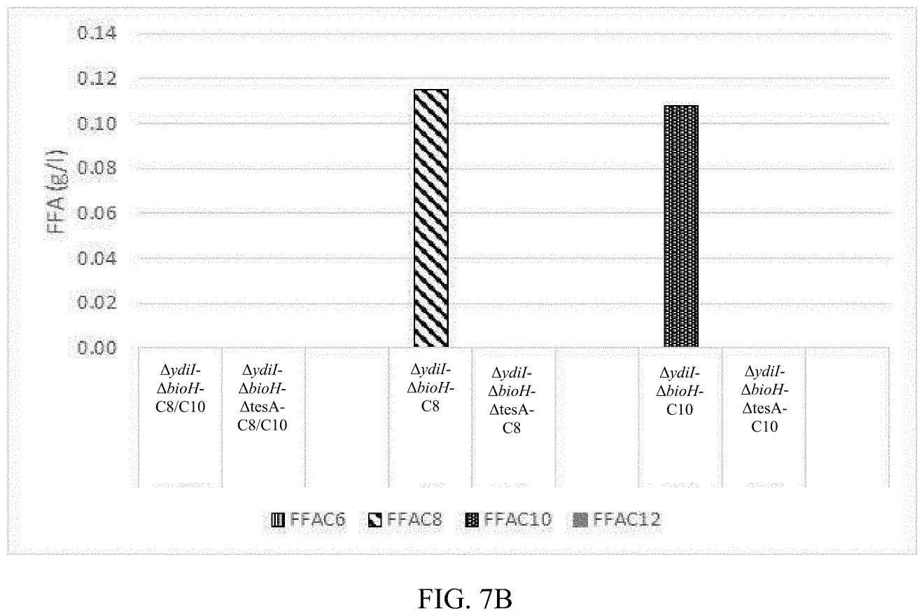

Each transformed deletion strain (including the control strains) was then grown as described above in the shake flask protocol and assayed for production of C6, C8, C10, C12 fatty acid methyl esters (FAMEs) and C6, C8, C10, and C12 free fatty acids (FFAs). The results for production of FAME are listed below in Table Y and FIG. 7A. The results for production of FFA are listed below in Table Z and FIG. 7B.

TABLE Y

| | Methyl | Methyl | Methyl | Methyl | total |
|---|---|---|---|---|---|
| | Hexanoate | Octanoate | Decanoate | Dodecanoate | FAME |
| Strain | (C6) g/L | (C8) g/L | (C10) g/L | (C12) g/L | g/l |
| ΔydiI-ΔbioH-C8/C10 | 0.7 | 6.8 | 12.1 | 0.1 | 19.75 |
| ΔydiI-ΔbioH-ΔtesA C8/C10 | 1.4 | 10.4 | 11.4 | 0.1 | 23.30 |
| ΔydiI-ΔbioH-C8 | 0.3 | 9.5 | 2.2 | 0.1 | 12.20 |
| ΔydiI-ΔbioH-ΔtesA-C8 | 0.6 | 12.9 | 2.2 | 0.1 | 15.80 |
| ΔydiI-ΔbioH-C10 | 1.3 | 0.1 | 20.8 | 0.1 | 22.23 |
| ΔydiI-ΔbioH-ΔtesA C10 | 1.2 | 0.1 | 20.4 | 0.1 | 21.82 |

TABLE Z

| | Butyric | Hexanoic | Octanoic | Decanoic | Dodecanoic | total FFA |
|---|---|---|---|---|---|---|
| Strain | acid (C4) g/l | acid (C6) g/l | acid (C8) g/l | acid (C10) g/l | acid (C12) g/l | g/l |
| ΔydiI-ΔbioH- | 0.0 | 0.0 | 0.0 | 0.0 | 0.0 | 0.000 |
| C8/C10 | | | | | | |
| ΔydiI-ΔbioH- | 0.0 | 0.0 | 0.0 | 0.0 | 0.0 | 0.000 |
| ΔtesA C8/C10 | | | | | | |
| ΔydiI-ΔbioH- | 0.0 | 0.0 | 0.1 | 0.0 | 0.0 | 0.115 |
| C8 | | | | | | |
| ΔydiI-ΔbioH- | 0.0 | 0.0 | 0.0 | 0.0 | 0.0 | 0.000 |
| ΔtesA C8 | | | | | | |
| ΔydiI-ΔbioH- | 0.1 | 0.0 | 0.0 | 0.1 | 0.0 | 0.254 |
| C10 | | | | | | |
| ΔydiI-ΔbioH- | 0.2 | 0.0 | 0.0 | 0.0 | 0.0 | 0.153 |
| ΔtesA C10 | | | | | | |

As shown in Table Y and FIG. 7A, the ΔydiI-ΔbioH-ΔtesA-C8 strain showed increased total FAME production compared to the ΔydiI-ΔbioH-C8 strain. The ΔydiI-ΔbioH-ΔtesA-C8/C10 strain showed increased total FAME production compared to the ΔydiI-ΔbioH-C8/C10 strain. The ΔydiI-ΔbioH-ΔtesA-C10 strain showed similar total FAME production compared to the ΔydiI-ΔbioH-C10 strain.

As shown in Table Z and FIG. 7B, the ΔydiI-ΔbioH-ΔtesA-C8 strain showed decreased total FFA production compared to the ΔydiI-ΔbioH-C8 strain. The ΔydiI-ΔbioH-C8 strain produced primarily C8 FFA and the ΔydiI-ΔbioH-ΔtesA-C8 strain produced no total FFA that was detectable at the scale of the experiment. The ΔydiI-ΔbioH-ΔtesA-C10 strain showed decreased total FFA production compared to the ΔydiI-ΔbioH-C10 strain. The ΔydiI-ΔbioH-C10 strain produced primarily C10 FFA and the ΔydiI-ΔbioH-ΔtesA-C10 strain produced no total FFA that was detectable at the scale of the experiment. This example shows that the ΔydiI-ΔbioH-ΔtesA-C8 strain and the ΔydiI-ΔbioH-ΔtesA-C8/C10 strain increased FAME production when compared to similar strains without the tesA deletion. This example also shows that the ΔydiI-ΔbioH-ΔtesA-C8 strain and the ΔydiI-ΔbioH-ΔtesA-C8/C10 strain decreased FFA production when compared to similar strains without the tesA deletion.

### Example H

An ACS enzyme was overexpressed in Recombinant Strain 1 and the influence of the overexpression of the ACS enzyme on FAME production and FFA production was measured. Type 2 plasmids were genetically engineered to express ACS enzyme (SEQ ID NO: 30) using different promoters as listed below in Table AA. The constitutive promoters, Pscp3, Pscp4, PtpiA, and PrpiA were used. The low phosphate inducible promoters PpstSIH and PphoE were also used. Type 1 plasmid was then genetically engineered to express C8-specific Asch, Asch(T184I, F236L, V268A, V296A, V317A, S328G). ACS expression strains were then prepared by transforming Recombinant Strain 1 with one of the Type 2 plasmids and the C8-specific Asch Type 1 plasmid. The ACS expression strains are listed below in Table AA. As a control plasmid, a Type 2 plasmid was genetically engineered to express rudolph (SEQ ID NO: 43) and used to prepare a control strain.

TABLE AA

| | Promoter used to | |
|---|---|---|
| | express ACS from | Asch variant expressed by |
| Strain | Type 2 plasmid | Type 1 plasmid |
| Pscp3-ACSM1 | Pscp3 | Asch(T184I, F236L, V268A, V296A, |
| | | V317A, S328G) |
| Pscp4-ACSM1 | Pscp4 | Asch(T184I, F236L, V268A, V296A, |
| | | V317A, S328G) |
| PtpiA-ACSM1 | PtpiA | Asch(T184I, F236L, V268A, V296A, |
| | | V317A, S328G) |
| PrpiA-ACSM1 | PrpiA | Asch(T184I, F236L, V268A, V296A, |
| | | V317A, S328G) |
| PpstSIH-ACSM1 | PpstSIH | Asch(T184I, F236L, V268A, V296A, |
| | | V317A, S328G) |
| PphoE-ACSM1 | PphoE | Asch(T184I, F236L, V268A, V296A, |
| | | V317A, S328G) |
| Control, PphoE-Rudolph | PphoE | Asch(T184I, F236L, V268A, V296A, |
| | | V317A, S328G) |
| Control (no Type 2 plasmid) | none | Asch(T184I, F236L, V268A, V296A, |
| | | V317A, S328G) |

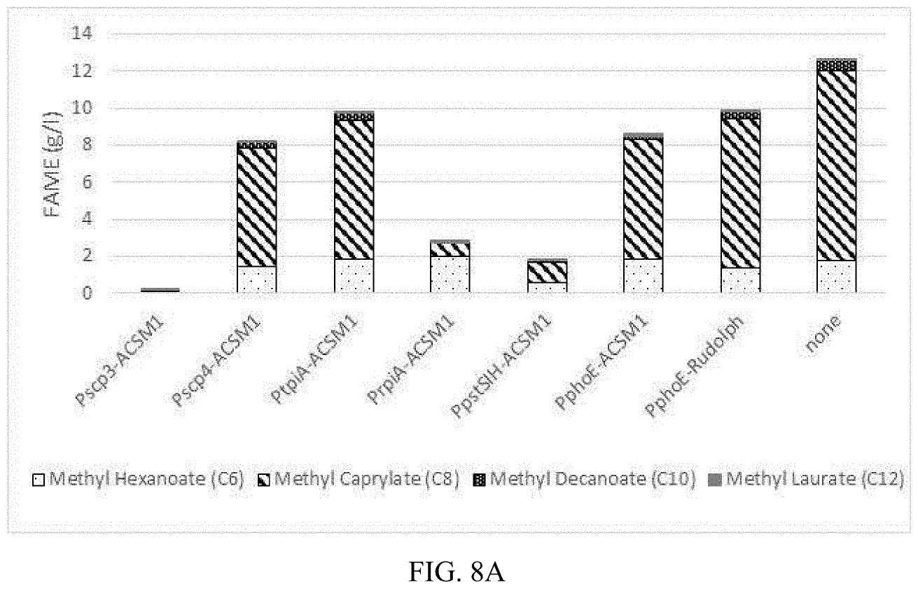

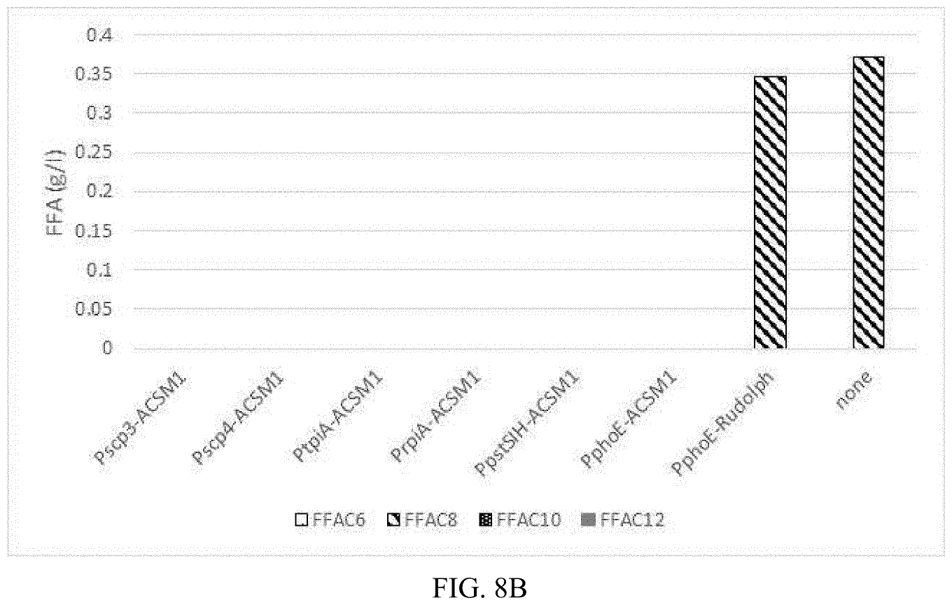

Each transformed ACS strain (including the control strains) were then grown as described above in the shake flask protocol and assayed for production of C6, C8, C10, C12 fatty acid methyl esters (FAMEs) and C4, C6, C8, C10, and C12 free fatty acids (FFAs). The results for production of FAME are listed below in Table BB and FIG. 8A. The results for production of FFA are listed below in Table CC and FIG. 8B.

TABLE BB

| | Methyl | Methyl | Methyl | Methyl | total |
|---|---|---|---|---|---|
| | Hexanoate | Octanoate | Decanoate | Dodecanoate | FAME |
| ACS Strain | (C6) g/L | (C8) g/L | (C10) g/L | (C12) g/L | g/l |
| Pscp3-ACSM1 | 0.1 | 0.1 | 0.0 | 0.1 | 0.2 |
| Pscp4-ACSM1 | 1.5 | 6.4 | 0.3 | 0.1 | 8.2 |
| PtpiA-ACSM1 | 1.8 | 7.6 | 0.4 | 0.1 | 9.8 |
| PrpiA-ACSM1 | 2.0 | 0.7 | 0.1 | 0.1 | 2.8 |
| PpstSIH-ACSM1 | 0.6 | 1.1 | 0.1 | 0.1 | 1.9 |
| PphoE-ACSM1 | 1.8 | 6.5 | 0.2 | 0.1 | 8.6 |
| Control, PphoE-Rudolph | 1.4 | 8.1 | 0.4 | 0.1 | 9.9 |
| Control, none | 1.8 | 10.3 | 0.5 | 0.1 | 12.6 |

TABLE CC

| | Butyric | Hexanoic | Octanoic | Decanoic | Dodecanoic | total FFA |
|---|---|---|---|---|---|---|
| Strain | acid (C4) g/l | acid (C6) g/l | acid (C8) g/l | acid (C10) g/l | acid (C12) g/l | g/l |
| Pscp3-ACSM1 | 0.0 | 0.0 | 0.0 | 0.0 | 0.0 | 0.0 |
| Pscp4-ACSM1 | 0.0 | 0.0 | 0.0 | 0.0 | 0.0 | 0.0 |
| PtpiA-ACSM1 | 0.0 | 0.0 | 0.0 | 0.0 | 0.0 | 0.0 |
| PrpiA-ACSM1 | 0.0 | 0.0 | 0.0 | 0.0 | 0.0 | 0.0 |
| PpstSIH-ACSM1 | 0.0 | 0.0 | 0.0 | 0.0 | 0.0 | 0.0 |
| PphoE-ACSM1 | 0.0 | 0.0 | 0.0 | 0.0 | 0.0 | 0.0 |
| Control, PphoE- | 0.0 | 0.0 | 0.3 | 0.0 | 0.0 | 0.3 |
| Rudolph | | | | | | |
| Control, none | 0.0 | 0.0 | 0.4 | 0.0 | 0.0 | 0.4 |

As shown in Table BB and FIG. 8A, under the conditions of the experiment, none of the ACS strains showed increased total FAME production compared to the control strains. As shown in Table CC and FIG. 8B, each of the ACS strains showed decreased total FFA production compared to the control strains. Each of the ACS strains produced no FFA that was detectable at the scale of the experiment. This example shows that overexpression of ACS can decrease total FFA production.

### Example I

An ACS enzyme was overexpressed in Recombinant Strain 1 with ydiI and bioH deletions and the influence of the overexpression of the ACS enzyme on FAME production and FFA production was measured. A Recombinant strain 1 was prepared with ydiI and bioH deletions as described above and then genetically engineered by chromosomally integrating ACS (SEQ ID NO: 44) under the control of the constitutive Pscp4 promoter. A Recombinant strain 1 was prepared with ydiI and bioH deletions as described above and then genetically engineered by chromosomally integrating ACS (SEQ ID NO: 44) under the control of the constitutive PtpiA promoter. A Recombinant strain 1 was prepared with ydiI and bioH deletions as described above as a control. Each of the strains with chromosomally integrated ACS, along with the control strain, were transformed with a Type 1 plasmid with either an inducible C8-specific Asch, Asch(T184I, F236L, V268A, V296A, V317A, S328G) or an inducible C10-specific Asch, Asch(V296A). The strains are listed below in Table DD.

TABLE DD

| | Constitutive | |
|---|---|---|
| | promoter used | Asch variant expressed by |
| Strain | to express ACS | Type 1 plasmid |
| ΔydiI-ΔbioH-C8 | — | Asch(T184I, F236L, |
| | | V268A, V296A, V317A, S328G) |
| ΔydiI-ΔbioH-C8 Pscp4-ACS | Pscp4 | Asch(T184I, F236L, |
| | | V268A, V296A, V317A, S328G) |
| ΔydiI-ΔbioH-C8 PtpiA-ACS | PtpiA | Asch(T184I, F236L, |
| | | V268A, V296A, V317A, S328G) |
| ΔydiI-ΔbioH-C10 | — | Asch(V296A) |
| ΔydiI-ΔbioH-C10 Pscp4-ACS | Pscp4 | Asch(V296A) |
| ΔydiI-ΔbioH-C10 PtpiA-ACS | PtpiA | Asch(V296A) |

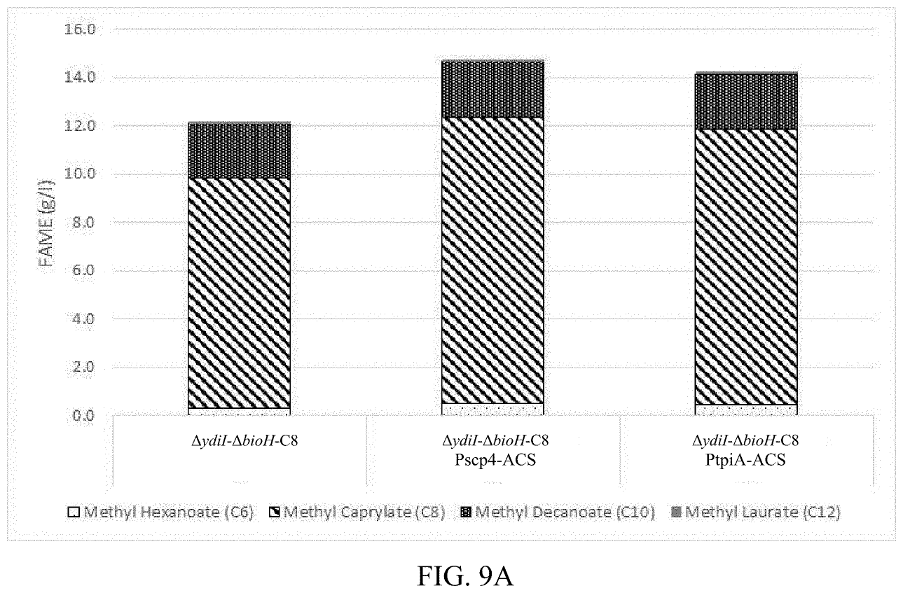

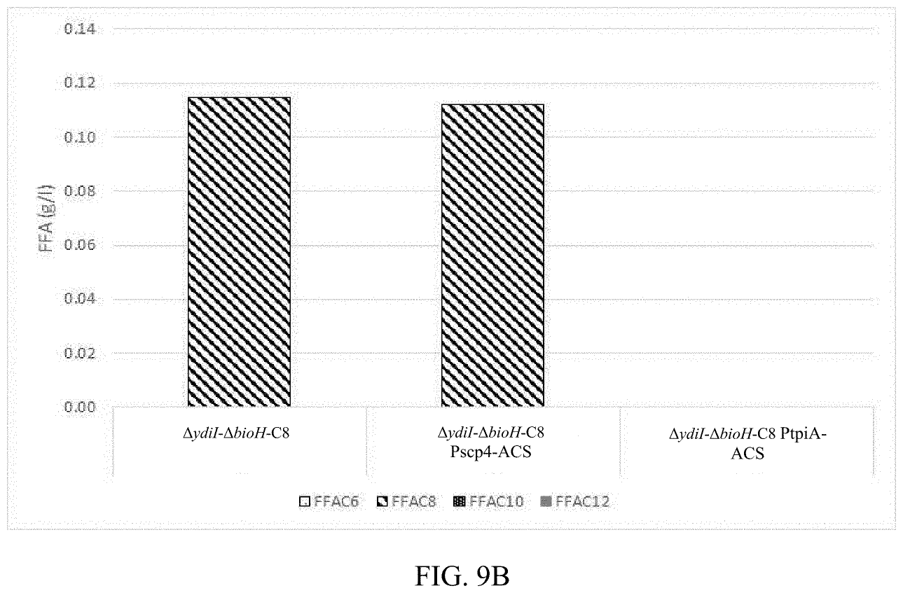

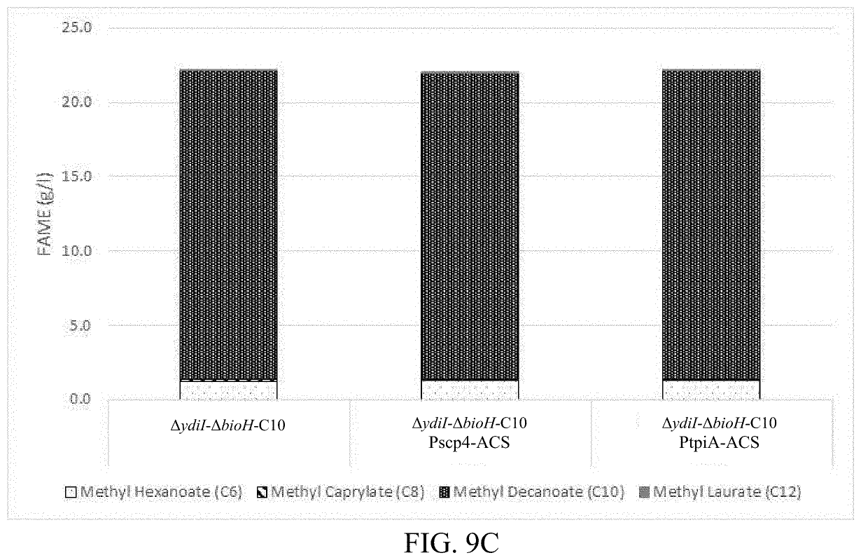

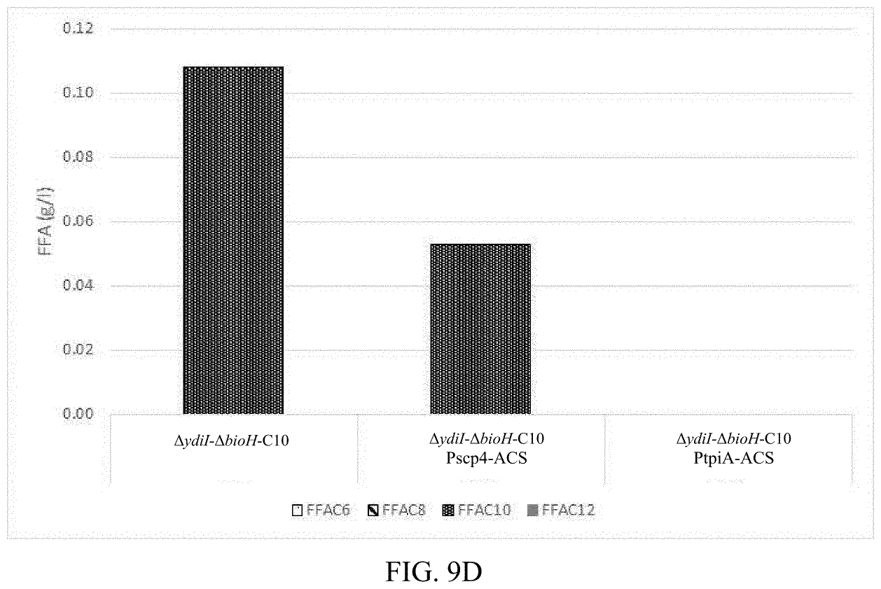

Each transformed ACS strain (and the control strains) was then grown as described above in the shake flask protocol and assayed for production of C6, C8, C10, C12 fatty acid methyl esters (FAMEs) and C4, C6, C8, C10, and C12 free fatty acids (FFAs). The results for production of FAME are listed below in Table EE and FIGS. 9A and 9C. The results for production of FFA are listed below in Table FF and FIGS. 9B and 9D.

TABLE EE

| | Methyl | Methyl | Methyl | Methyl | total |
|---|---|---|---|---|---|
| | Hexanoate | Octanoate | Decanoate | Dodecanoate | FAME |
| ACS Strain | (C6) g/L | (C8) g/L | (C10) g/L | (C12)g/L | g/l |
| ΔydiI-ΔbioH-C8 | 0.3 | 9.5 | 2.2 | 0.1 | 12.2 |
| ΔydiI-ΔbioH-C8 | 0.5 | 11.9 | 2.3 | 0.1 | 14.8 |
| Pscp4-ACS | | | | | |
| ΔydiI-ΔbioH-C8 | 0.5 | 11.4 | 2.3 | 0.1 | 14.3 |
| PtpiA-ACS | | | | | |
| ΔydiI-ΔbioH-C10 | 1.3 | 0.1 | 20.8 | 0.1 | 22.2 |
| ΔydiI-ΔbioH-C10 | 1.3 | 0.1 | 20.6 | 0.1 | 22.1 |
| Pscp4-ACS | | | | | |
| ΔydiI-ΔbioH-C10 | 1.3 | 0.1 | 20.7 | 0.1 | 22.2 |
| PtpiA-ACS | | | | | |

TABLE FF

| | Butyric | Hexanoic | Octanoic | Decanoic | Dodecanoic | total FFA |
|---|---|---|---|---|---|---|
| Strain | acid (C4) g/l | acid (C6) g/l | acid (C8) g/l | acid (C10) g/l | acid (C12) g/l | g/l |
| Δydil-ΔbioH-C8 | 0.0 | 0.0 | 0.1 | 0.0 | 0.0 | 0.1 |
| Δydil-ΔbioH-C8 | 0.0 | 0.0 | 0.1 | 0.0 | 0.0 | 0.1 |
| Pscp4-ACS | | | | | | |
| Δydil-ΔbioH-C8 | 0.0 | 0.0 | 0.0 | 0.0 | 0.0 | 0.0 |
| PtpiA-ACS | | | | | | |
| Δydil-ΔbioH-C10 | 0.1 | 0.0 | 0.0 | 0.1 | 0.0 | 0.3 |
| Δydil-ΔbioH-C10 | 0.1 | 0.0 | 0.0 | 0.1 | 0.0 | 0.2 |
| Pscp4-ACS | | | | | | |
| Δydil-ΔbioH-C10 | 0.1 | 0.0 | 0.0 | 0.0 | 0.0 | 0.1 |
| PtpiA-ACS | | | | | | |

As shown in Table EE and FIG. 9A, the ΔydiI-ΔbioH-C8 Pscp4-ACS and ΔydiI-ΔbioH-C8 PtpiA-ACS showed increased total FAME production compared to the ΔydiI-ΔbioH-C8 control strain. As shown in Table FF and FIG. 9B, the ΔydiI-ΔbioH-C8 Pscp4-ACS strain showed similar total FFA production compared to the ΔydiI-ΔbioH-C8 strain. The ΔydiI-ΔbioH-C8 PtpiA-ACS strain showed substantially decreased total FFA production compared to the ΔydiI-ΔbioH-C8 control strain. The ΔydiI-ΔbioH-C8 PtpiA-ACS strain showed no FFA that was detectable at the scale of the experiment. As shown in Table EE and FIG. 9C, the ΔydiI-ΔbioH-C10 Pscp4-ACS strain and the ΔydiI-ΔbioH-C10 PtpiA-ACS strain showed similar total FAME production compared to the ΔydiI-ΔbioH-C10 control strain. As shown in Table FF and FIG. 9D, the ΔydiI-ΔbioH-C10 Pscp4-ACS strain and the ΔydiI-ΔbioH-C10 PtpiA-ACS strain showed decreased total FFA production compared to the ΔydiI-ΔbioH-C10 control strain. The ΔydiI-ΔbioH-C10 PtpiA-ACS strain showed no FFA that was detectable at the scale of the experiment.

This example shows that overexpression of ACS can increase total FAME production in a ΔydiI and ΔbioH background with a C8-specific Asch. Overexpression of ACS can reduce total FFA production in a ΔydiI and ΔbioH background with a C8-specific Asch depending on the expression of ACS. Overexpression of ACS can reduce total FFA production in a ΔydiI and ΔbioH background with a C10-specific Asch.

### Example J

The Cbac enzyme was overexpressed in a ΔydiI-ΔbioH background and the influence of the ΔydiI and ΔbioH background on total FAME production and FFA production was measured. Strains were prepared with the ΔydiI-ΔbioH background and with inducible Cbac. Recombinant Strain 1 with deleted ydiI and bioH was used. Recombinant Strain 1 with deleted ydiI and bioH and without the PpstSIH-npht7-ter-TT-loxP cassette was also used (and without deletion of the native aldehyde-alcohol dehydrogenase adhE). Type 3A Plasmid (without NphT7(LSVA) expression) and Type 3B Plasmid (with NphT7(LSVA) expression) were used as described above. Type 4 Plasmid (with Cbac(V223A, I246L)) was also used. The strains are listed below in Table GG.

TABLE GG

| | | nphT7-ter | nphT7 | Cbac |
|---|---|---|---|---|
| | Host strain | expression | (LSVA) | (V223A, I246L) |
| Strain | deletion | cassette | expression | expression |
| ΔydiI-ΔbioH | ΔydiI-ΔbioH | Yes | No | Yes |
| nphT7-ter | | | | |
| Cbac | | | | |
| ΔydiI-ΔbioH | ΔydiI-ΔbioH | Yes | Yes | Yes |
| nphT7-ter | | | | |
| nphT7(LSVA) | | | | |
| Cbac | | | | |
| ΔydiI-ΔbioH | ΔydiI-ΔbioH | No | No | Yes |
| Cbac | | | | |
| ΔydiI-ΔbioH | ΔydiI-ΔbioH | No | Yes | Yes |
| nphT7(LSVA) | | | | |
| Cbac | | | | |
| nphT7-ter | — | Yes | No | Yes |
| Cbac | | | | |
| nphT7-ter | — | Yes | Yes | Yes |
| nphT7(LSVA) | | | | |
| Cbac | | | | |
| Cbac | — | No | No | Yes |
| nphT7(LSVA) | — | No | Yes | Yes |
| Cbac | | | | |

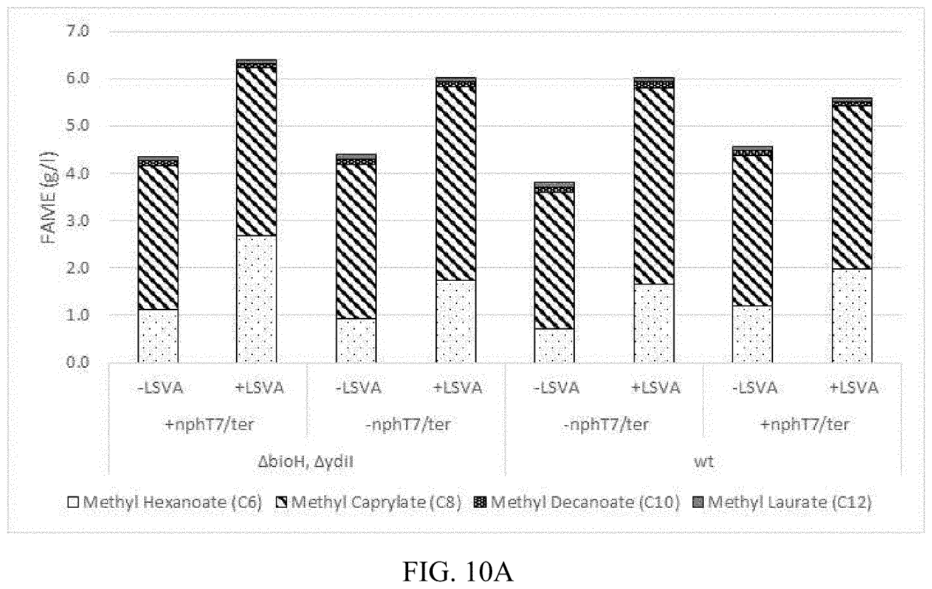

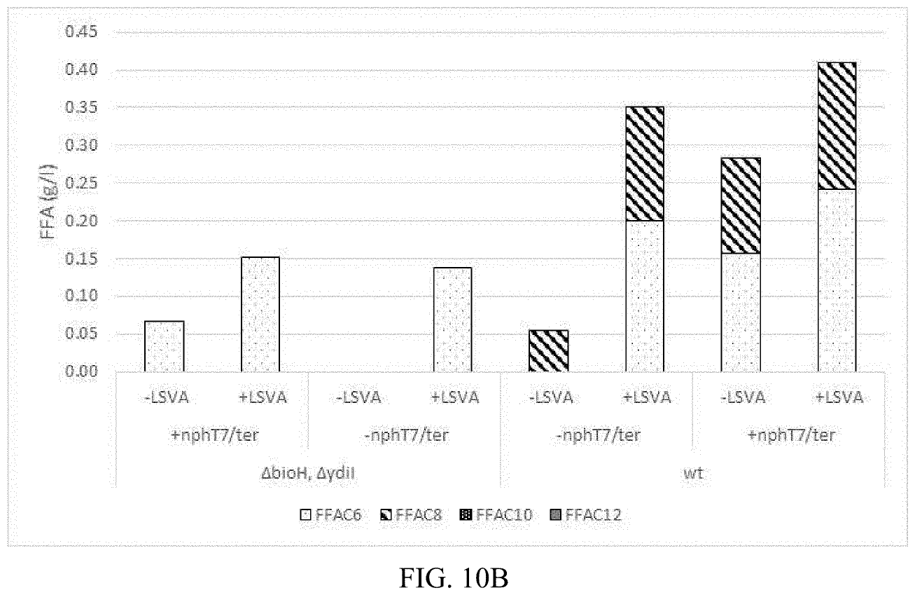

Each prepared strain (and the control strains) were then grown as described above in the shake flask protocol and assayed for production of C6, C8, C10, C12 fatty acid methyl esters (FAME) and C6, C8, C10, and C12 free fatty acids (FFA) at 48 h. The results for production of FAME are listed below in Table HH and FIG. 10A. The results for production of FFA are listed below in Table II and FIG. 10B.

TABLE HH

| | Methyl | Methyl | Methyl | Methyl | total |
|---|---|---|---|---|---|
| | Hexanoate | Octanoate | Decanoate | Dodecanoate | FAME |
| Strain | (C6) g/L | (C8)g/L | (C10) g/L | (C12)g/L | g/l |
| ΔydiI-ΔbioH; | 1.1 | 3.1 | 0.1 | 0.1 | 4.4 |
| nphT7-ter; Cbac | | | | | |
| ΔydiI-ΔbioH; | 2.7 | 3.6 | 0.1 | 0.1 | 6.4 |
| nphT7-ter; | | | | | |
| nphT7(LSVA); Cbac | | | | | |
| ΔydiI-ΔbioH; Cbac | 0.9 | 3.3 | 0.1 | 0.1 | 4.4 |
| ΔydiI-ΔbioH; | 1.7 | 4.1 | 0.1 | 0.1 | 6.0 |
| nphT7(LSVA); Cbac | | | | | |
| nphT7-ter; Cbac | 0.7 | 2.9 | 0.1 | 0.1 | 3.8 |
| nphT7-ter; nphT7(LSVA); Cbac | 1.7 | 4.1 | 0.1 | 0.1 | 6.0 |
| Cbac | 1.2 | 3.2 | 0.1 | 0.1 | 4.6 |
| nphT7(LSVA); Cbac | 2.0 | 3.4 | 0.1 | 0.1 | 5.6 |

TABLE II

| | Hexanoic | Octanoic | Decanoic | Dodecanoic | total |
|---|---|---|---|---|---|
| | acid (C6) | acid (C8) | acid (C10) | acid (C12) | FFA |
| Strain | g/l | g/l | g/l | g/l | g/l |
| ΔydiI-ΔbioH; | 0.1 | 0.0 | 0.0 | 0.0 | 0.1 |
| nphT7-ter; | | | | | |
| Cbac | | | | | |
| ΔydiI-ΔbioH; | 0.2 | 0.0 | 0.0 | 0.0 | 0.2 |
| nphT7-ter; | | | | | |
| nphT7(LSVA); | | | | | |
| Cbac | | | | | |
| ΔydiI-ΔbioH; | 0.0 | 0.0 | 0.0 | 0.0 | 0.0 |
| Cbac | | | | | |
| ΔydiI-ΔbioH; | 0.1 | 0.0 | 0.0 | 0.0 | 0.1 |
| nphT7(LSVA); | | | | | |
| Cbac | | | | | |
| nphT7-ter; | 0.0 | 0.1 | 0.0 | 0.0 | 0.1 |
| Cbac | | | | | |
| nphT7-ter; | 0.2 | 0.1 | 0.0 | 0.0 | 0.4 |
| nphT7(LSVA); | | | | | |
| Cbac | | | | | |
| Cbac | 0.2 | 0.1 | 0.0 | 0.0 | 0.3 |
| nphT7(LSVA); | 0.2 | 0.2 | 0.0 | 0.0 | 0.4 |
| Cbac | | | | | |

As shown in Table HH and FIG. 10A, under the conditions of the experiment, the levels of FAME produced in the strains with the ΔydiI-ΔbioH background appear to be slightly increased compared to the levels of FAME produced in the strains without the ydiI and bioH deletions. As shown in Table II and FIG. 10B, the levels of FFA production in the strains with the ΔydiI-ΔbioH background are substantially decreased compared to the strains without the ydiI and bioH deletions. The strains with the ydiI and bioH deletions produced only C6 FFA compared to the strains without the ydiI and bioH deletions that produced a mix of C6 FFA and C8 FFA. The ΔydiI-ΔbioH Cbac strain (without the nphT7-ter cassette and without nphT7(LSVA)) produced no FFA that was detectable at the scale of the experiment. This experiment shows that the ydiI and bioH deletions can decrease and/or eliminate FFA production in FAME producing strains.

SEQUENCES

| SEQ | Sequence | |
|---|---|---|
| ID NO: | Name | Sequence |
| 1 | yjjU | MRAQFNPFDLYLGTSAGAQNLSAFICNQPGYARKVIMRYTTKR |
| | | EFFDPLRFVRGGNLIDLDWLVEATASQMPLQMDTAARLFDSGK |
| | | SFYMCACRQDDYAPNYFLPTKQNWLDVIRASSAIPGFYRSGVS |
| | | LEGINYLDGGISDAIPVKEAARQGAKTLVVIRTVPSQMYYTPQ |
| | | WFKRMERWLGDSSLQPLVNLVQHHETSYRDIQQFIEKPPGKLR |
| | | IFEIYPPKPLHSIALGSRIPALREDYKLGRLCGRYFLATVGKL |
| | | LTEKAPLTRHLVPVVTPESIVIPPAPVANDTLVAEVSDAPQAN |
| | | DPTFNNEDLA |
| 2 | ybaW | MQTQIKVRGYHLDVYQHVNNARYLEFLEEARWDGLENSDSFQW |
| | | MTAHNIAFVVVNININYRRPAVLSDLLTITSQLQQLNGKSGIL |
| | | SQVITLEPEGQVVADALITFVCIDLKTQKALALEGELREKLEQ |
| | | MVK |
| 3 | frmB | MELIEKHVSF GGWQNMYRHY SQSLKCEMNV GVYLPPKAAN |
| | | EKLPVLYWLS GLTCNEQNFI TKSGMQRYAA EHNIIVVAPD |
| | | TSPRGSHVAD ADRYDLGQGA GFYLNATQAP WNEHYKMYDY |
| | | IRNELPDLVM HHFPATAKKS ISGHSMGGLG ALVLALRNPD |
| | | EYVSVSAFSP IVSPSQVPWG QQAFAAYLAE NKDAWLDYDP |
| | | VSLISQGQRV AEIMVDQGLS DDFYAEQLRT PNLEKICQEM |
| | | NIKTLIRYHE GYDHSYYFVS SFIGEHIAYH ANKLNMR |
| 4 | ydiI | MIWKRKITLEALNAMGEGNMVGFLDIRFEHIGDDTLEATMPVD |
| | | SRTKQPFGLLHGGASVVLAESIGSVAGYLCTEGEQKVVGLEIN |
| | | ANHVRSAREGRVRGVCKPLHLGSRHQVWQIEIFDEKGRLCCSS |
| | | RLTTAIL |
| 5 | bioH | MNNIWWQTKGQGNVHLVLLHGWGLNAEVWRCIDEELSSHFTLH |
| | | LVDLPGFGRSRGFGALSLADMAEAVLQQAPDKAIWLGWSLGGL |
| | | VASQIALTHPERVQALVTVASSPCFSARDEWPGIKPDVLAGFQ |
| | | QQLSDDFQRTVERFLALQTMGTETARQDARALKKTVLALPMPE |
| | | VDVLNGGLEILKTVDLRQPLQNVSMPFLRLYGYLDGLVPRKVV |
| | | PMLDKLWPHSESYIFAKAAHAPFISHPAEFCHLLVALKQRV |
| 6 | tesA | MMNFNNVFRWHLPFLFLVLLTFRAAAADTLLILGDSLSAGYRM |
| | | SASAAWPALLNDKWQSKTSVVNASISGDTSQQGLARLPALLKQ |
| | | HQPRWVLVELGGNDGLRGFQPQQTEQTLRQILQDVKAANAEPL |
| | | LMQIRLPANYGRRYNEAFSAIYPKLAKEFDVPLLPFFMEEVYL |
| | | KPQWMQDDGIHPNRDAQPFIADWMAKQLQPLVNHDS |
| 7 | ACS | MWNDHDSPEEFNFASDVLDYWAQMEEEGKRGPSPAFWWVNGQG |
| | | DEIKWSFRKLRDLTCRTANVFEQICGLQQGDHLALILPRVPEW |
| | | WLVTVGCMRTGIIFMPGTTQLKAKDILYRIQISRAKAIVTTAS |
| | | LVPEVESVASECPDLKTKLVVSDHSHEGWLDFCSLIKSASPDH |
| | | TCIKSKMKDPMAIFFTSGTTGYPKMAKHNQGLAFRSYIPSCRK |
| | | LLKLKTSDILWCMSDPGWILATVGCLIEPWTSGCTVFIHHLPQ |
| | | FDPKVIVEVLFKYPITQCLAAPGVYRMVLQQKTSNLRFPTLEH |
| | | CTTGGESLLPEEYEQWKQRTGLSIHEVYGQSETGISSATLREM |
| | | KIKRGSIGKAILPFDLQIIDEKGNILPPNTEGYIGIRIKPTRP |
| | | LGLFMEYENSPESTSEVECGDFYNSGDRATIDEEGYIWFLGRG |
| | | DDVINASGYRIGPVEVENALAEHPAVAESAVVSSPDKDRGEVV |
| | | KAFIVLNPEFLSHDQEQLIKELQHHVKSVTAPYKYPRKVEFVS |
| | | ELPKTVTGKIKRKELRNKEFGQL |
| 8 | fadB, | MSLQGKVALVTGASRGIGQAIALELGRLGAVVIGTATSASGAE |
| | Pseudomonas | KIAETLKANGVEGAGLVLDVSSDESVAATLEHIQQHLGQPLIV |
| | aeruginosa | VNNAGITRDNLLVRMKDDEWFDVVNTNLNSLYRLSKAVLRGMT |
| | | KARWGRIINIGSVVGAMGNAGQTNYAAAKAGLEGFTRALAREV |
| | | GSRAITVNAVAPGFIDTDMTRELPEAQREALLGQIPLGRLGQA |
| | | EEIAKVVGFLASDGAAYVTGATVPVNGGMYMS |
| 9 | fadG, | MSLQGKVALVTGASRGIGQAIALELGRLGAVVIGTATSASGAE |
| | Pseudomonas | KIAETLKANGVEGAGLVLDVSSDESVAATLEHIQQHLGQPLIV |
| | aeruginosa | VNNAGITRDNLLVRMKDDEWFDVVNTNLNSLYRLSKAVLRGMT |
| | | KARWGRIINIGSVVGAMGNAGQTNYAAAKAGLEGFTRALAREV |
| | | GSRAITVNAVAPGFIDTDMTRELPEAQREALLAQIPLGRLGQA |
| | | EEIAKVVGFLASDGAAYVTGATVPVNGGMYMS |
| 10 | Hbd, | MKKIFVLGAGTMGAGIVQAFAQKGCEVIVRDIKEEFVDRGIAG |
| | Clostridium | ITKGLEKQVAKGKMSEEDKEAILSRISGTTDMKLAADCDLVVE |
| | beijerinckii | AAIENMKIKKEIFAELDGICKPEAILASNTSSLSITEVASATK |
| | | RPDKVIGMHFFNPAPVMKLVEIIKGIATSQETFDAVKELSVAI |
| | | GKEPVEVAEAPGFVVNGILIPMINEASFILQEGIASVEDIDTA |
| | | MKYGANHPMGPLALGDLIGLDVCLAIMDVLFTETGDNKYRASS |
| | | ILRKYVRAGWLGRKSGKGFYDYSK |
| 11 | Crt, | MELNNVILEKEGKVAVVTINRPKALNALNSDTLKEMDYVIGEI |
| | Clostridium | ENDSEVLAVILTGAGEKSFVAGADISEMKEMNTIEGRKFGILG |
| | acetobutylicum | NKVFRRLELLEKPVIAAVNGFALGGGCEIAMSCDIRIASSNAR |
| | | FGQPEVGLGITPGFGGTQRLSRLVGMGMAKQLIFTAQNIKADE |
| | | ALRIGLVNKVVEPSELMNTAKEIANKIVSNAPVAVKLSKQAIN |
| | | RGMQCDIDTALAFESEAFGECFSTEDQKDAMTAFIEKRKIEGF |
| | | KNR |
| 12 | Ech, | MSDTEVPVLAEVRNRVGHLALNRPVGLNALTLQMIRITWRQLH |
| | Pseudomonas | AWESDPEIVAVVLRANGEKAFCAGGDIRSLYDSYQAGDDLHHV |
| | putida | FLEEKYSLDQYIHGYPKPIVALMDGFVLGGGMGLVQGTALRVV |
| | | TERVKMGMPETSIGYFPDVGGSYFLPRLPGELGLYLGITGIQI |
| | | RAADALYARLADWCLPSERISEFDRRLDQISWGYAPREILAGL |
| | | LSSLASNRLLGAELKSLHPAIDEHFTQPDLSAIRASLQAERRP |
| | | EYQDWAEQTVELLNNRSPLAMSATLKLLRLGRTLSLANCFELE |
| | | LHLERQWFAKGDLIEGVRALLIDKDKTPRWNPPTLEQLDTNRV |
| | | NEFFDGFQPAT |
| 13 | Ter, | MIVKPMVRNNICLNAHPQGCKKGVEDQIEYTKKRITAEVKAGA |
| | Treponema | KAPKNVLVLGCSNGYGLASRITAAFGYGAATIGVSFEKAGSET |
| | denticola | KYGTPGWYNNLAFDEAAKREGLYSVTIDGDAFSDEIKAQVIEE |
| | | AKKKGIKFDLIVYSLASPVRTDPDTGIMHKSVLKPFGKTFTGK |
| | | TVDPFTGELKEISAEPANDEEAAATVKVMGGEDWEVGSNS |
| 14 | Npht7 | MTDVRFRIIGTGAYVPERIVSNDEVGAPAGVDDDWITRKTGIR |
| | | QRRWAADDQATSDLATAAGRAALKAAGITPEQLTVIAVATSTP |
| | | DRPQPPTAAYVQHHLGATGTAAFDVNAVCSGTVFALSSVAGTL |
| | | VYRGGYALVIGADLYSRILNPADRKTVVLFGDGAGAMVLGPTS |
| | | TGTGPIVRRVALHTFGGLTDLIRVPAGGSRQPLDTDGLDAGLQ |
| | | YFAMDGREVRRFVTEHLPQLIKGFLHEAGVDAADISHFVPHQA |
| | | NGVMLDEVFGELHLPRATMHRTVETYGNTGAASIPITMDAAVR |
| | | AGSFRPGELVLLAGFGGGMAASFALIEW |
| 15 | Npht7 100 | MTDVRFRIIG TGAYVPERIV SNDEVGAPAG VDDDWITRKT |
| | is H, amino | GIRQRRWAAD DQATSDLATA AGRAALKAAG ITPEQLTVIA |
| | acid 147 is | VATSTPDRPQ PPTAAYVQHH LGATGTAAFD VNAVCSGTVF |
| | V, S or T, | ALSSVAGTLV YRGGYALVIG ADLYSRXLNP ADRKTVVLFG |
| | amino acid | DGAGAMVLGP TSTGTGPIVR RVALHTFGGL TDLIRVPAGG |
| | 217 is F | SRQPLDTDGL DAGLQYFAMD GREVRRFVTE HLPQLIKGFL |
| | and amino | HEAGVDAADI SHFVPHQANG VMLDEVFGEL HLPRATMHRT |
| | acid 323 is | VETYGNTGAA SIPITMDAAV RAGSFRPGEL VLLAGFGGGM |
| | A | AAAFALIEW |
| 16 | Npht7 H100L | MTDVRFRIIG TGAYVPERIV SNDEVGAPAG VDDDWITRKT |
| | substitution, | GIRQRRWAAD DQATSDLATA AGRAALKAAG ITPEQLTVIA |
| | an | VATSTPDRPQ PPTAAYVQHL LGATGTAAFD VNAVCSGTVF |
| | I147V, | ALSSVAGTLV YRGGYALVIG ADLYSRXLNP ADRKTVVLFG |
| | I147S or | DGAGAMVLGP TSTGTGPIVR RVALHTFGGL TDLIRVPAGG |
| | 11471 amino | SRQPLDTDGL DAGLQYVAMD GREVRRFVTE HLPQLIKGFL |
| | acid | HEAGVDAADI SHFVPHQANG VMLDEVFGEL HLPRATMHRT |
| | substitution, | VETYGNTGAA SIPITMDAAV RAGSFRPGEL VLLAGFGGGM |
| | an F217V | AAAFALIEW |
| | substitution | |
| | and an | |
| | S323A | |
| 17 | Npht7 100 | MTDVRFRIIG TGAYVPERIV SNDEVGAPAG VDDDWITRKT |
| | is H or L, | GIRQRRWAAD DQATSDLATA AGRAALKAAG ITPEQLTVIA |
| | amino acid | VATSTPDRPQ PPTAAYVQHX LGATGTAAFD VNAVCSGTVF |
| | residue 147 | ALSSVAGTLV YRGGYALVIG ADLYSRXLNP ADRKTVVLFG |
| | is S, T or | DGAGAMVLGP TSTGTGPIVR RVALHTFGGL TDLIRVPAGG |
| | V, amino | SRQPLDTDGL DAGLQYFAMD GREVRRFVTE HLPQLIKGFL |
| | acid | HEAGVDAADI SHFVPHQANG VMLDEVFGEL XLPRATMHRT |
| | residue 271 | VETYGNTGAA SIPITMDAAV RAGSFRPGEL VLLAGFGGGM |
| | is F or V | AAAFALIEW |
| | and amino | |
| | acid | |
| | residue 323 | |
| | is A | |
| 18 | Asch, wt, | MGIRITGTGLFHPTDFITNEELVESLNAYVEQYNLENADKIAA |
| | Acinetobacter | GEIEELRGSSAEFIEKASGIKRRYVAEKTGILDPKRLRPLLHE |
| | schindleri | RSNDELSIQAEWGVAAAKQAMENAGVTAEDIDVVILSCSNIQR |
| | CIP 107287 | AYPALAIEIQTALGIQGYAYDMNVACSAATFGIKQAADAIKSG |
| | | ARRVLMVNVEITSGHTDFRSRDCHFIFGDVATASIIEETETKT |
| | | GFEIEDIELFTQFSNNIRNNFGYLNLSEVDADIDNNRFRQDGR |
| | | KVFKEVCPLVAKMITKQLEKNQIEPTNVKRFWLHQANVNMNEL |
| | | ILKLIVGKEHAKSELVPLILDEFANTSSAGVIIALHRTANEVN |
| | | DGEYGVLCSFGAGYSVGSILVKKHVA |
| 19 | Asch T184I, | MGIRITGTGLFHPTDFITNEELVESLNAYVEQYNLENADKIAA |
| | F236L, | GEIEELRGSSAEFIEKASGIKRRYVAEKTGILDPKRLRPLLHE |
| | V268A, | RSNDELSIQAEWGVAAAKQAMENAGVTAEDIDVVILSCSNIQR |
| | V296A, | AYPALAIEIQTALGIQGYAYDMNVACSAATFGIKQAADAIKSG |
| | V317A, and | ARRVLMVNVEIISGHTDFRSRDCHFIFGDVATASIIEETETKT |
| | S328G | GFEIEDIELFTQFSNNIRNNLGYLNLSEVDADIDNNRFRQDGR |
| | | KVFKEVCPLAAKMITKQLEKNQIEPTNVKRFWLHQANANMNEL |
| | | ILKLIVGKEHAKSELAPLILDEFANTGSAGVIIALHRTANEVN |
| | | DGEYGVLCSFGAGYSVGSILVKKHVA |
| 20 | Asch T184I, | MGIRITGTGLFHPTDFITNEELVESLNAYVEQYNLENADKIAA |
| | V296A, and | GEIEELRGSSAEFIEKASGIKRRYVAEKTGILDPKRLRPLLHE |
| | V268A | RSNDELSIQAEWGVAAAKQAMENAGVTAEDIDVVILSCSNIQR |
| | | AYPALAIEIQTALGIQGYAYDMNVACSAATFGIKQAADAIKSG |
| | | ARRVLMVNVEIISGHTDFRSRDCHFIFGDVATASIIEETETKT |
| | | GFEIEDIELFTQFSNNIRNNFGYLNLSEVDADIDNNRFRQDGR |
| | | KVFKEVCPLAAKMITKQLEKNQIEPTNVKRFWLHQANANMNEL |
| | | ILKLIVGKEHAKSELVPLILDEFANTSSAGVIIALHRTANEVN |
| | | DGEYGVLCSFGAGYSVGSILVKKHVA |
| 21 | Asch V296A | MGIRITGTGLFHPTDFITNEELVESLNAYVEQYNLENADKIAA |
| | | GEIEELRGSSAEFIEKASGIKRRYVAEKTGILDPKRLRPLLHE |
| | | RSNDELSIQAEWGVAAAKQAMENAGVTAEDIDVVILSCSNIQR |
| | | AYPALAIEIQTALGIQGYAYDMNVACSAATFGIKQAADAIKSG |
| | | ARRVLMVNVEITSGHTDFRSRDCHFIFGDVATASIIEETETKT |
| | | GFEIEDIELFTQFSNNIRNNFGYLNLSEVDADIDNNRFRQDGR |
| | | KVFKEVCPLVAKMITKQLEKNQIEPTNVKRFWLHQANANMNEL |
| | | ILKLIVGKEHAKSELVPLILDEFANTSSAGVIIALHRTANEVN |
| | | DGEYGVLCSFGAGYSVGSILVKKHVA |
| 22 | Hche, | MTPLSPVDQIFLWLEKRQQPMHVGGLHIFSFPDDADAKYMTEL |
| | Hahella | AQQLRAYATPQAPFNRRLRQRWGRYYWDTDAQFDLEHHFRHEA |
| | chejuensis | LPKPGRIRELLAHVSAEHSNLMDRERPMWECHLIEGIRGRRFA |
| | | VYYKAHHCMLDGVAAMRMCVKSYSFDPTATEMPPIWAISKDVT |
| | | PARETQAPAAGDLVHSLSQLVEGAGRQLATVPTLIRELGKNLL |
| | | KARDDSDAGLIFRAPPSILNQRITGSRRFAAQSYALERFKAIG |
| | | KAFQATVNDVVLAVCGSALRNYLLSRQALPDQPLIAMAPMSIR |
| | | QDDSDSGNQIAMILANLGTHIADPVRRLELTQASARESKERFR |
| | | QMTPEEAVNYTALTLAPSGLNLLTGLAPKWQAFNVVISNVPGP |
| | | NKPLYWNGARLEGMYPVSIPVDYAALNITLVSYRDQLEFGFTA |
| | | CRRTLPSMQRLLDYIEQGIAELEKAAGV |
| 23 | fused E. | MSWIERIKSNITPTRKASIPEGVWTKCDSCGQVLYRAELERNL |
| | coli accD | EVCPKCDHHMRMTARNRLHSLLDEGSLVELGSELEPKDVLKFR |
| | subunit and | DSKKYKDRLASAQKETGEKDALVVMKGTLYGMPVVAAAFEFAF |
| | accA | MGGSMGSVVGARFVRAVEQALEDNCPLICFSASGGARMQEALM |
| | subunit | SLMQMAKTSAALAKMQERGLPYISVLTDPTMGGVSASFAMLGD |
| | enzyme | LNIAEPKALIGFAGPRVIEQTVREKLPPGFQRSEFLIEKGAID |
| | | MIVRRPEMRLKLASILAKLMNLPAPNPEAPREGVVVPPVPDQE |
| | | PEALSGGGGSGGGGSGGGGSGGGGSAAASLNFLDFEQPIAELE |
| | | AKIDSLTAVSRQDEKLDINIDEEVHRLREKSVELTRKIFADLG |
| | | AWQIAQLARHPQRPYTLDYVRLAFDEFDELAGDRAYADDKAIV |
| | | GGIARLDGRPVMIIGHQKGRETKEKIRRNFGMPAPEGYRKALR |
| | | LMQMAERFKMPIITFIDTPGAYPGVGAEERGQSEAIARNLREM |
| | | SRLGVPVVCTVIGEGGSGGALAIGVGDKVNMLQYSTYSVISPE |
| | | GCASILWKSADKAPLAAEAMGIIAPRLKELKLIDSIIPEPLGG |
| | | AHRNPEAMAASLKAQLLADLADLDVLSTEDLKNRRYQRLMSYG |
| | | YA |
| 24 | pstSIH | aaatcagact gaagacttta tctctctgtc ataaaactgt |
| | promoter | catattcctt acatataact gtcacctgtt |
| | | tgtcctattt tgcttctcgt agccaacaaa caatgcttta |
| | | tgaatcctcc c |
| 25 | phoE | gatcttgata tcaaacgaac gttttagcag gactgtcgtc |
| | promoter | ggttgccaac catctgcgag caaagcatgg cgttttgttg |
| | | cgcgggatca gcaagcctag cggcagttgt ttacgctttt |
| | | attacagatt taataaatta ccacatttta agaatattat |
| | | taatctgtaa tatatcttta acaatctcag gttaaaaact |
| | | ttcctgtttt caacg |
| 26 | tpiA | ggtttgaata aatgacaaaa agcaaagcct ttgtgccgat |
| | promoter | gaatctctat actgtttcac a |
| 27 | E. coli | MDIRKIKKLIELVEESGISELEISEGEESVRISRAAPAASFPV |
| | accB | MQQAYAAPMMQQPAQSNAAAPATVPSMEAPAAAEISGHIVRSP |
| | | MVGTFYRTPSPDAKAFIEVGQKVNVGDTLCIVEAMKMMNQIEA |
| | | DKSGTVKAILVESGQPVEFDEPLVVIE |
| 28 | E. coli | MLDKIVIANRGEIALRILRACKELGIKTVAVHSSADRDLKHVL |
| | accC | LADETVCIGPAPSVKSYLNIPAIISAAEITGAVAIHPGYGFLS |
| | | ENANFAEQVERSGFIFIGPKAETIRLMGDKVSAIAAMKKAGVP |
| | | CVPGSDGPLGDDMDKNRAIAKRIGYPVIIKASGGGGGRGMRVV |
| | | RGDAELAQSISMTRAEAKAAFSNDMVYMEKYLENPRHVEIQVL |
| | | ADGQGNAIYLAERDCSMQRRHQKVVEEAPAPGITPELRRYIGE |
| | | RCAKACVDIGYRGAGTFEFLFENGEFYFIEMNTRIQVEHPVTE |
| | | MITGVDLIKEQLRIAAGQPLSIKQEEVHVRGHAVECRINAEDP |
| | | NTFLPSPGKITRFHAPGGFGVRWESHIYAGYTVPPYYDSMIGK |
| | | LICYGENRDVAIARMKNALQELIIDGIKTNVDLQIRIMNDENF |
| | | QHGGTNIHYLEKKLGLQEK |
| 29 | E. coli | gagttaacca cgcggcttgc caacggggtc tgaatcgctt |
| | rpiA | tttttgtata taatgcgtgt |
| | promoter | |
| 30 | mouse ACSM1 | MWNDHDSPEEFNFASDVLDYWAQMEEEGKRGPSPAFWWVNGQG |
| | | DEIKWSFRKLRDLTCRTANVFEQICGLQQGDHLALILPRVPEW |
| | | WLVTVGCMRTGIIFMPGTTQLKAKDILYRIQISRAKAIVTTAS |
| | | LVPEVESVASECPDLKTKLVVSDHSHEGWLDFCSLIKSASPDH |
| | | TCIKSKMKDPMAIFFTSGTTGYPKMAKHNQGLAFRSYIPSCRK |
| | | LLKLKTSDILWCMSDPGWILATVGCLIEPWTSGCTVFIHHLPQ |
| | | FDPKVIVEVLFKYPITQCLAAPGVYRMVLQQKTSNLRFPTLEH |
| | | CTTGGESLLPEEYEQWKQRTGLSIHEVYGQSETGISSATLREM |
| | | KIKRGSIGKAILPFDLQIIDEKGNILPPNTEGYIGIRIKPTRP |
| | | LGLFMEYENSPESTSEVECGDFYNSGDRATIDEEGYIWFLGRG |
| | | DDVINASGYRIGPVEVENALAEHPAVAESAVVSSPDKDRGEVV |
| | | KAFIVLNPEFLSHDQEQLIKELQHHVKSVTAPYKYPRKVEFVS |
| | | ELPKTVTGKIKRKELRNKEFGQL |
| 31 | Clostridiales | MTTRIIGTGSYVPEQIVTNNDLAQIVETNDEWIRSRTGIGERR |
| | bacterium | IATTESTSYMAANAAMRALEQSGVKPEEIDLILLGTSSPDYCF |
| | 1_7_47_FAA | PNGACEVQGMIGAVNAACYDISAACTGFVYALNTAHAFISSGI |
| | 3-ketoacyl- | YKTALVIGSDVLSKLIDWTDRGTCVLFGDGAGAVVVKADETGI |
| | CoA | LGINMHSDGTKGNVLTCGSRTNGNFLLGKKPELGYMTMDGQEV |
| | synthase | FKFAVRKAPECIKQVLDDAGVAAAEVRYFVLHQANYRIIESIA |
| | variant | KRLKVSVDCFPVNMEHYGNTSGASVPLLLDEINRKGMLESGDK |
| | (V223A, | IVFSGFGAGLTWGATLLEW |
| | I246L) | |
| 32 | cheB | MSKIRVLSVDDSALMRQIMTEIINSHSDMEMVATAPDPLVARD |
| | | LIKKFNPDVLTLDVEMPRMDGLDFLEKLMRLRPMPVVMVSSLT |
| | | GKGSEVTLRALELGAIDFVTKPQLGIREGMLAYNEMIAEKVRT |
| | | AAKASLAAHKPLSAPTTLKAGPLLSSEKLIAIGASTGGTEAIR |
| | | HVLQPLPLSSPALLITQHMPPGFTRSFADRLNKLCQIGVKEAE |
| | | DGERVLPGHAYIAPGDRHMELSRSGANYQIKIHDGPAVNRHRP |
| | | SVDVLFHSVAKQAGRNAVGVILTGMGNDGAAGMLAMRQAGAWT |
| | | LAQNEASCVVFGMPREAINMGGVCEVVDLSQVSQQMLAKISAG |
| | | QAIRI |
| 33 | yigL | MYQVVASDLDGTLLSPDHTLSPYAKETLKLLTARGINFVFATG |
| | | RHHVDVGQIRDNLEIKSYMITSNGARVHDLDGNLIFAHNLDRD |
| | | IASDLFGVVNDNPDIITNVYRDDEWFMNRHRPEEMRFFKEAVF |
| | | QYALYEPGLLEPEGVSKVFFTCDSHEQLLPLEQAINARWGDRV |
| | | NVSFSTLTCLEVMAGGVSKGHALEAVAKKLGYSLKDCIAFGDG |
| | | MNDAEMLSMAGKGCIMGSAHQRLKDLHPELEVIGTNADDAVPH |
| | | YLRKLYLS |
| 34 | ybgC | VNTTLFRWPVRVYYEDTDAGGVVYHASYVAFYERARTEMLRHH |
| | | HFSQQALMAERVAFVVRKMTVEYYAPARLDDMLEIQTEITSMR |
| | | GTSLVFTQRIVNAENTLLNEAEVLVVCVDPLKMKPRALPKSIV |
| | | AEFKQ |
| 35 | yfcE | MKLMFASDIHGSLPATERVLELFAQSGAQWLVILGDVLNHGPR |
| | | NALPEGYAPAKVAERLNEVAHKVIAVRGNCDSEVDQMLLHFPI |
| | | TAPWQQVLLEKQRLFLTHGHLFGPENLPALNQNDVLVYGHTHL |
| | | PVAEQRGEIFHFNPGSVSIPKGGNPASYGMLDNDVLSVIALND |
| | | QSIIAQVAINP |
| 36 | yeiG | MEMLEEHRCFEGWQQRWRHDSSTLNCPMTFSIFLPPPRDHTPP |
| | | PVLYWLSGLTCNDENFTTKAGAQRVAAELGIVLVMPDTSPRGE |
| | | KVANDDGYDLGQGAGFYLNATQPPWATHYRMYDYLRDELPALV |
| | | QSQFNVSDRCAISGHSMGGHGALIMALKNPGKYTSVSAFAPIV |
| | | NPCSVPWGIKAFSSYLGEDKNAWLEWDSCALMYASNAQDAIPT |
| | | LIDQGDNDQFLADQLQPAVLAEAARQKAWPMTLRIQPGYDHSY |
| | | YFIASFIEDHLRFHAQYLLK |
| 37 | yiiD | MSQLPGLSRETRESIAMYHLRVPQTEEELERYYQFRWEMLRKP |
| | | LHQPKGSERDAWDAMAHHQMVVDEQGNLVAVGRLYINADNEAS |
| | | IRFMAVHPDVQDKGLGTLMAMTLESVARQEGVKRVTCSAREDA |
| | | VEFFAKLGFVNQGEITTPTTTPIRHFLMIKPVATLDDILHRGD |
| | | WCAQLQQAWYEHIPLSEKMGVRIQQYTGQKFITTMPETGNQNP |
| | | HHTLFAGSLFSLATLTGWGLIWLMLRERHLGGTIILADAHIRY |
| | | SKPISGKPHAVADLGALSGDLDRLARGRKARVQMQVEIFGDET |
| | | PGAVFEGTYIVLPAKPFGPYEEGGNEEE |
| 38 | yqiA | MSTLLYLHGFNSSPRSAKASLLKNWLAEHHPDVEMIIPQLPPY |
| | | PSDAAELLESIVLEHGGDSLGIVGSSLGGYYATWLSQCFMLPA |
| | | VVVNPAVRPFELLTDYLGQNENPYTGQQYVLESRHIYDLKVMQ |
| | | IDPLEAPDLIWLLQQTGDEVLDYRQAVAYYASCRQTVIEGGNH |
| | | AFTGFEDYFNPIVDFLGLHHL |
| 39 | ybfF | MKLNIRAQTA QNQHNNSPIV LVHGLFGSLD NLGVLARDLV |
| | | NDHNIIQVDM RNHGLSPRDP VMNYPAMAQD LVDTLDAQQI |
| | | DKATFIGHSM GGKAVMALTA LASDRIDKLV AIDIAPVDYH |
| | | VRRHDEIFAA INAVSESDAQ TRQQAAAIMR QHLNEEGVIQ |
| | | FLLKSFVDGE WRFNVPVLWD QYPHIVGWEK IPAWDHPALF |
| | | IPGGNSPYVS EQYRDDLLAQ FPQARAHVIA GAGHWVHAEK |
| | | PDAVLRAIRR YLND |
| 40 | ydiI | atgATATGGA AACGGAAAAT CACCCTGGAA GCACTGAATG |
| | | CTATGGGTGA AGGAAACATG GTGGGGTTCC TGGATATTCG |
| | | CTTTGAACAT ATTGGTGATG ACACCCTTGA AGCGACAATG |
| | | CCAGTAGACT CGCGGACAAA GCAGCCTTTC GGGTTGCTGC |
| | | ATGGAGGAGC ATCCGTGGTA CTGGCCGAAA GTATCGGTTC |
| | | CGTTGCCGGT TATTTATGTA CCGAAGGTGA GCAAAAAGTG |
| | | GTTGGTCTGG AAATCAATGC TAACCACGTC CGCTCGGCAC |
| | | GAGAAGGGCG GGTGCGCGGC GTATGCAAAC CGTTGCATCT |
| | | CGGTTCGCGT CACCAGGTCT GGCAGATTGA AATCTTCGAT |
| | | GAGAAAGGGC GTTTGTGCTG TTCGTCACGA TTGACGACCG |
| | | CCATTTTGtg a |
| 41 | bioH | atgAATAACA TCTGGTGGCA GACCAAAGGT CAGGGGAATG |
| | | TTCATCTTGT GCTGCTGCAC GGATGGGGAC TGAATGCCGA |
| | | AGTGTGGCGT TGCATTGACG AGGAACTTAG CTCGCATTTT |
| | | ACGCTGCACC TTGTTGACCT GCCCGGCTTC GGGCGTAGCC |
| | | GGGGATTTGG TGCGCTGTCA CTTGCTGATA TGGCCGAAGC |
| | | CGTGCTGCAA CAGGCACCTG ATAAAGCCAT TTGGTTAGGC |
| | | TGGAGTCTGG GCGGGCTGGT GGCAAGCCAG ATTGCGTTAA |
| | | CCCATCCCGA GCGTGTTCAG GCGCTGGTCA CCGTGGCGTC |
| | | GTCACCTTGT TTTAGTGCTC GTGACGAGTG GCCGGGGATA |
| | | AAACCGGACG TGCTGGCGGG ATTTCAGCAG CAACTCAGTG |
| | | ATGATTTTCA GCGTACAGTG GAGCGGTTCC TGGCGTTACA |
| | | AACCATGGGG ACTGAAACGG CGCGCCAGGA TGCGCGGGCG |
| | | TTGAAGAAAA CCGTTCTGGC GTTACCGATG CCGGAGGTTG |
| | | ACGTGCTTAA TGGCGGGCTG GAAATCCTGA AAACGGTCGA |
| | | TCTCCGTCAG CCGCTGCAAA ACGTGTCCAT GCCGTTTTTG |
| | | CGATTGTATG GCTATCTCGA CGGTCTGGTG CCGCGCAAAG |
| | | TGGTGCCGAT GCTGGATAAA CTTTGGCCTC ACAGCGAATC |
| | | ATATATCTTC GCCAAAGCGG CCCATGCGCC ATTTATTTCG |
| | | CATCCGGCCG AGTTTTGTCA CCTGCTGGTG GCGTTGAAGC |
| | | AGAGGGTGta g |
| 42 | tesA | atgATGAACT TCAACAATGT TTTCCGCTGG CATTTGCCCT |
| | | TCCTGTTCCT GGTCCTGTTA ACCTTCCGTG CCGCCGCAGC |
| | | GGACACGTTA TTGATTCTGG GTGATAGCCT GAGCGCCGGG |
| | | TATCGAATGT CTGCCAGCGC GGCCTGGCCT GCCTTGTTGA |
| | | ATGATAAGTG GCAGAGTAAA ACGTCGGTAG TTAATGCCAG |
| | | CATCAGCGGC GACACCTCGC AACAAGGACT GGCGCGCCTT |
| | | CCGGCTCTGC TGAAACAGCA TCAGCCGCGT TGGGTGCTGG |
| | | TTGAACTGGG CGGCAATGAC GGTTTGCGTG GTTTTCAGCC |
| | | ACAGCAAACC GAGCAAACGC TGCGCCAGAT TTTGCAGGAT |
| | | GTCAAAGCCG CCAACGCTGA ACCATTGTTA ATGCAAATAC |
| | | GTCTGCCTGC AAACTATGGT CGCCGTTATA ATGAAGCCTT |
| | | TAGCGCCATT TACCCCAAAC TCGCCAAAGA GTTTGATGTT |
| | | CCGCTGCTGC CCTTTTTTAT GGAAGAGGTC TACCTCAAGC |
| | | CACAATGGAT GCAGGATGAC GGTATTCATC CCAACCGCGA |
| | | CGCCCAGCCG TTTATTGCCG ACTGGATGGC GAAGCAGTTG |
| | | CAGCCTTTAG TAAATCATGA CTCAtaa |
| 43 | rudolph | MSLSKQVLPHDVKMRYHMDGCVNGHSFTIEGEGAGKPYEGKKI |
| | | LELRVTKGGPLPFAFDILSSVFTYGNRCFCEYPEDMPDYFKQS |
| | | LPEGHSWERTLMFEDGGCGTASAHISLDKNCFVHKSTFHGVNF |
| | | PANGPVMQKKAMNWEPSSELITACDGILKGDVTMFLLLEGGHR |
| | | LKCQFTTSYKAHKAVKMPPNHIIEHVLVKKEVADGFQIQEHAV |
| | | AKHFTVDVKET |
| 44 | ACS | ATGTGGAATGACCATGATTCACCTGAGGAGTTTAACTTTGCAA |
| | | GTGATGTCCTGGACTACTGGGCTCAAATGGAGGAGGAGGGCAA |
| | | GAGAGGACCAAGTCCAGCCTTTTGGTGGGTGAATGGCCAAGGA |
| | | GATGAAATAAAGTGGAGCTTCAGGAAGCTGAGGGACCTCACCT |
| | | GTCGCACTGCCAACGTCTTTGAGCAGATTTGTGGCCTGCAGCA |
| | | AGGAGATCACCTGGCCTTGATTCTGCCCCGAGTGCCCGAGTGG |
| | | TGGTTGGTGACAGTGGGCTGCATGCGAACAGGGATCATCTTCA |
| | | TGCCTGGGACTACCCAACTGAAAGCCAAGGACATTCTCTACCG |
| | | AATACAAATATCTCGAGCCAAAGCCATTGTGACCACAGCTAGC |
| | | CTTGTCCCAGAGGTGGAATCTGTGGCTTCTGAGTGTCCTGATC |
| | | TGAAAACCAAGCTGGTGGTGTCTGATCACAGCCATGAAGGGTG |
| | | GCTTGATTTCTGTTCACTGATTAAATCAGCATCCCCAGACCAT |
| | | ACTTGTATTAAGTCAAAGATGAAGGATCCCATGGCCATCTTCT |
| | | TCACCAGTGGGACCACAGGCTACCCCAAGATGGCAAAGCACAA |
| | | CCAGGGACTTGCCTTCCGGTCATATATCCCTTCATGCAGAAAA |
| | | TTATTGAAGCTGAAGACATCTGACATCTTGTGGTGCATGTCAG |
| | | ACCCAGGATGGATTCTGGCTACCGTGGGGTGCCTGATCGAGCC |
| | | ATGGACATCAGGATGTACAGTCTTCATCCACCACCTCCCTCAA |
| | | TTCGACCCCAAAGTCATTGTAGAGGTACTGTTCAAATACCCCA |
| | | TCACTCAGTGCCTTGCTGCCCCAGGCGTGTATCGAATGGTTCT |
| | | TCAGCAGAAAACCTCCAACCTCAGGTTCCCCACCCTTGAGCAT |
| | | TGCACTACTGGTGGGGAGAGCCTGCTGCCTGAGGAGTATGAGC |
| | | AGTGGAAGCAAAGGACAGGTCTTTCCATCCACGAGGTCTATGG |
| | | ACAGTCAGAAACGGGGATCAGCAGTGCCACCCTCCGGGAAATG |
| | | AAGATCAAGCGAGGCTCCATAGGGAAGGCCATCTTACCCTTTG |
| | | ACTTGCAGATCATCGATGAAAAGGGCAACATCCTCCCACCCAA |
| | | CACTGAAGGATACATTGGCATCAGGATCAAGCCCACCAGGCCT |
| | | CTAGGCCTCTTCATGGAATATGAGAATAGCCCAGAGAGCACAT |
| | | CTGAAGTGGAGTGTGGGGACTTTTACAATAGTGGGGATAGAGC |
| | | GACCATTGATGAAGAGGGCTACATCTGGTTCTTGGGAAGGGGC |
| | | GATGATGTCATCAATGCTTCCGGGTATCGCATCGGGCCTGTAG |
| | | AGGTGGAGAACGCCTTGGCGGAGCACCCAGCAGTGGCAGAGTC |
| | | TGCGGTGGTGAGCAGCCCGGACAAGGATCGAGGAGAGGTGGTG |
| | | AAGGCGTTTATTGTCCTCAACCCAGAGTTCCTGTCACACGATC |
| | | AGGAACAGCTTATCAAAGAGCTACAGCATCATGTGAAGTCAGT |
| | | GACGGCACCATACAAGTACCCCAGGAAGGTGGAGTTTGTTTCA |
| | | GAATTGCCCAAAACTGTCACAGGCAAAATCAAAAGGAAGGAAC |
| | | TTCGAAACAAGGAGTTTGGTCAGCTATAG |

1. A recombinant cell for producing a fatty acid ester, wherein the recombinant cell is genetically engineered for reduced activity of a polypeptide with at least 80%, 85%, 90%, 95%, 98%, or 100% sequence identity to an amino acid sequence set forth in SEQ ID NO: 1-3,
wherein the recombinant cell is capable of producing at least 5 g/L total mono fatty acid ester.

2. The recombinant cell of claim 1, wherein the recombinant cell produces a reduction in total free fatty acids compared to a recombinant cell without reduced activity of the polypeptide with at least 80%, 85%, 90%, 95%, 98%, or 100% sequence identity to the amino acid sequence set forth in SEQ ID NO: 1-3.

3. The recombinant cell of claim 2, wherein the reduction in free fatty acids comprises a reduction in C10 free fatty acids.

4. The recombinant cell of claim 1, wherein the recombinant cell produces a decreased ratio of total free fatty acids to total fatty acid esters compared to a recombinant cell without reduced activity of the polypeptide with at least 80%, 85%, 90%, 95%, 98%, or 100% sequence identity to the amino acid sequence set forth in SEQ ID NO: 1-3.

5. The recombinant cell of claim 1, wherein the recombinant cell produces an increase in total fatty acid ester compared to a recombinant cell without reduced activity of the polypeptide with at least 80%, 85%, 90%, 95%, 98%, or 100% sequence identity to the amino acid sequence set forth in SEQ ID NO: 1-3.

6. The recombinant cell of claim 5, wherein the increase in fatty acid ester production comprises an increase in fatty acid ester of C10 chain length.

7. The recombinant cell of claim 1, wherein the recombinant cell produces a fatty acid ester of C4, C6, C8, and/or C10 chain length.

8. The recombinant cell of claim 1, wherein the recombinant cell produces a fatty acid ester of C8 and/or C10 chain length.

9. The recombinant cell of any one of claims 1-8, wherein the alkoxy group of the fatty acid ester is derived from a C1, C2, C3, or C4 monoalcohol.

10. The recombinant cell of claim 9, wherein the alkoxy group of the fatty acid ester is derived from a C1 or C2 monoalcohol.

11. The recombinant cell of claim 10, wherein the monoalcohol is methanol

12. The recombinant cell of any one of claims 1-11, further comprising one or more nucleic acids selected from the group consisting of:
a heterologous nucleic acid segment encoding a polypeptide with enoyl-CoA reductase activity;
a heterologous nucleic acid segment encoding a polypeptide with bifunctional 3-hydroxyacyl-CoA dehydrogenase/dehydratase activity;
a heterologous nucleic acid segment encoding a polypeptide with ester synthase activity;
a heterologous nucleic acid segment encoding an enzyme with acetyl-CoA carboxylase activity; and
a heterologous nucleic acid segment encoding a polypeptide with ketoacyl-CoA synthase activity.

13. The recombinant cell of claim 12, wherein the polypeptide with ketoacyl-CoA synthase activity is an NphT7 polypeptide.

14. The recombinant cell of claim 12, wherein the polypeptide with ketoacyl-CoA synthase activity is an NphT7 variant selected from the group consisting of NphT7(I147S, F217V) and NphT7(H100L, I147S, F217V S323A).

15. The recombinant cell of claim 12, wherein the polypeptide with ketoacyl-CoA synthase activity is a wild type Asch.

16. The recombinant cell of claim 12, wherein the polypeptide with ketoacyl-CoA synthase activity is an Asch variant selected from the group consisting of Asch (T184I, F236L, V268A, V296A, V317A, S328G); Asch (T184I, V296A, V268A); and Asch (V296A).

17. The recombinant cell of any one of the preceding claims, wherein the recombinant cell is a fungal cell, or a bacterial cell.

18. The recombinant cell of any one of the preceding claims, wherein the recombinant cell is Escherichia coli.

19. The recombinant cell of any one of the preceding claims, wherein the recombinant cell is yeast.

20. A method for decreasing free fatty acid production, the method comprising culturing a recombinant cell, wherein:
the recombinant cell is genetically engineered for reduced activity of a polypeptide with at least 80%, 85%, 90%, 95%, 98%, or 100% sequence identity to an amino acid sequence set forth in SEQ ID NO: 1-3; and
the recombinant cell is capable of producing at least 5 g/L fatty acid ester.

21. The method of claim 20, wherein production of C10 free fatty acids is reduced.

22. The method of claim 20, wherein the recombinant cell produces a decreased ratio of total free fatty acids to total fatty acid esters compared to a recombinant cell without reduced activity of the polypeptide with at least 80%, 85%, 90%, 95%, 98%, or 100% sequence identity to the amino acid sequence set forth in SEQ ID NO: 1-3.

23. The method of claim 20, wherein the recombinant cell produces a fatty acid ester of C4, C6, C8, and/or C10 chain length.

24. The method of claim 20, wherein the recombinant cell produces a fatty acid ester of C8 and/or C10 chain length.

25. The method of any one of claims 20-24, wherein an alkoxy group of the fatty acid ester is derived from a C1, C2, C3, or C4 monoalcohol.

26. The method of claim 25, wherein the alkoxy group of the fatty acid ester is derived from a C1 or C2 monoalcohol.

27. The method of claim 26, wherein the monoalcohol is methanol.

28. The method of any one of claim 20-27, wherein the recombinant cell comprises one or more nucleic acids selected from the group consisting of:
a heterologous nucleic acid segment encoding a polypeptide with enoyl-CoA reductase activity;
a heterologous nucleic acid segment encoding a polypeptide with bifunctional 3-hydroxyacyl-CoA dehydrogenase/dehydratase activity;
a heterologous nucleic acid segment encoding a polypeptide with ester synthase activity;
a heterologous nucleic acid segment encoding an enzyme with acetyl-CoA carboxylase activity; and
a heterologous nucleic acid segment encoding a polypeptide with ketoacyl-CoA synthase activity.

29. The method of claim 28, wherein the polypeptide with ketoacyl-CoA synthase activity is an NphT7 polypeptide.

30. The method of claim 28, wherein the polypeptide with ketoacyl-CoA synthase activity is an NphT7 variant selected from the group consisting of NphT7(I147S, F217V) and NphT7(H100L, I147S, F217V S323A).

31. The method of claim 28, wherein the polypeptide with ketoacyl-CoA synthase activity is a wild type Asch.

32. The method of claim 28, wherein the polypeptide with ketoacyl-CoA synthase activity is an Asch variant selected from the group consisting of Asch (T184I, F236L, V268A, V296A, V317A, S328G); Asch (T184I, V296A, V268A); and Asch (V296A).

33. The method of any one of claims 20-32, wherein the recombinant cell is a fungal cell, or a bacterial cell.

34. The method of any one of claims 20-32, wherein the recombinant cell comprises an Escherichia coli.

35. The method of any one of claims 20-32, wherein the recombinant cell comprises a yeast cell.

36. A recombinant cell for producing a fatty acid ester, wherein the recombinant cell is genetically engineered for reduced activity of a polypeptide with at least 80%, 85%, 90%, 95%, 98%, or 100% sequence identity to an amino acid sequence set forth in SEQ ID NO: 4,
wherein the recombinant cell is capable of producing at least 5 g/L total fatty acid ester.

37. The recombinant cell of claim 36, wherein the recombinant cell produces a reduction in total free fatty acids compared to a recombinant cell without reduced activity of a polypeptide with at least 80%, 85%, 90%, 95%, 98%, or 100% sequence identity to an amino acid sequence set forth in SEQ ID NO: 4.

38. The recombinant cell of claim 37, wherein the reduction in free fatty acids comprises a reduction in C8 and/or C10 free fatty acids.

39. The recombinant cell of claim 36, wherein the recombinant cell produces a decreased ratio of total free fatty acids to total fatty acid esters compared to a recombinant cell without reduced activity of a polypeptide with at least 80%, 85%, 90%, 95%, 98%, or 100% sequence identity to an amino acid sequence set forth in SEQ ID NO: 4.

40. The recombinant cell of claim 36, wherein the recombinant cell produces an increase in fatty acid ester of C8 chain length compared to a recombinant cell without reduced activity of a polypeptide with at least 80%, 85%, 90%, 95%, 98%, or 100% sequence identity to an amino acid sequence set forth in SEQ ID NO: 4.

41. The recombinant cell of claim 36, wherein the recombinant cell produces a fatty acid ester of C4, C6, C8, and/or C10 chain length.

42. The recombinant cell of claim 36, wherein the recombinant cell produces a fatty acid ester of C8 and/or C10 chain length.

43. The recombinant cell of any one of claims 36-42, wherein the alkoxy group of the fatty acid ester is derived from a C1, C2, C3, or C4 monoalcohol.

44. The recombinant cell of claim 43, wherein the alkoxy group of the fatty acid ester is derived from a C1 or C2 monoalcohol.

45. The recombinant cell of claim 44, wherein the monoalcohol is methanol.

46. The recombinant cell of any one of claims 36-45, further comprising one or more nucleic acids selected from the group consisting of:
a heterologous nucleic acid segment encoding a polypeptide with enoyl-CoA reductase activity;
a heterologous nucleic acid segment encoding a polypeptide with bifunctional 3-hydroxyacyl-CoA dehydrogenase/dehydratase activity;
a heterologous nucleic acid segment encoding a polypeptide with ester synthase activity;
a heterologous nucleic acid segment encoding an enzyme with acetyl-CoA carboxylase activity; and
a heterologous nucleic acid segment encoding a polypeptide with ketoacyl-CoA synthase activity.

47. The recombinant cell of claim 46, wherein the polypeptide with ketoacyl-CoA synthase activity is an NphT7 polypeptide.

48. The recombinant cell of claim 46, wherein the polypeptide with ketoacyl-CoA synthase activity is an NphT7 variant selected from the group consisting of NphT7(I147S, F217V) and NphT7(H100L, I147S, F217V S323A).

49. The recombinant cell of claim 46, wherein the polypeptide with ketoacyl-CoA synthase activity is a wild type Asch.

50. The recombinant cell of claim 46, wherein the polypeptide with ketoacyl-CoA synthase activity is an Asch variant selected from the group consisting of Asch (T184I, F236L, V268A, V296A, V317A, S328G); Asch (T184I, V296A, V268A); and Asch (V296A).

51. The recombinant cell of any one of claims 36-50, wherein the recombinant cell is a fungal cell or a bacterial cell.

52. The recombinant cell of any one of claims 36-50, wherein the recombinant cell is an Escherichia coli.

53. The recombinant cell of any one of claims 36-50, wherein the recombinant cell is a yeast cell.

54. A method for decreasing free fatty acid production, the method comprising culturing a recombinant cell, wherein:
the recombinant cell is genetically engineered for reduced activity of a polypeptide with at least 80%, 85%, 90%, 95%, 98%, or 100% sequence identity to an amino acid sequence set forth in SEQ ID NO: 4; and
the recombinant cell is capable of producing at least 5 g/L total fatty acid ester.

55. The method of claim 54, wherein the reduction in free fatty acids comprises a reduction in C8 and/or C10 free fatty acids.

56. The method of claim 54, wherein the recombinant cell produces a decreased ratio of total free fatty acids to total fatty acid esters compared to a recombinant cell without reduced activity of a polypeptide with at least 80%, 85%, 90%, 95%, 98%, or 100% sequence identity to an amino acid sequence set forth in SEQ ID NO: 4.

57. The method of claim 54, wherein the recombinant cell produces an increase in fatty acid ester of C8 chain length compared to a recombinant cell without reduced activity of a polypeptide with at least 80%, 85%, 90%, 95%, 98%, or 100% sequence identity to an amino acid sequence set forth in SEQ ID NO: 4.

58. The method of claim 54, wherein the recombinant cell produces a fatty acid ester of C4, C6, C8, and/or C10 chain length.

59. The method of claim 54, wherein the recombinant cell produces a fatty acid ester of C8 and/or C10 chain length.

60. The method of any one of claims 54-59, wherein the alkoxy group of the fatty acid ester is derived from a C1, C2, C3, or C4 monoalcohol.

61. The method of claim 60, wherein the alkoxy group of the fatty acid ester is derived from a C1 or C2 monoalcohol.

62. The method of claim 61, wherein the monoalcohol is methanol.

63. The method of any one of claims 54-62, further comprising one or more nucleic acids selected from the group consisting of:
a heterologous nucleic acid segment encoding a polypeptide with enoyl-CoA reductase activity;
a heterologous nucleic acid segment encoding a polypeptide with bifunctional 3-hydroxyacyl-CoA dehydrogenase/dehydratase activity;
a heterologous nucleic acid segment encoding a polypeptide with ester synthase activity;
a heterologous nucleic acid segment encoding an enzyme with acetyl-CoA carboxylase activity; and
a heterologous nucleic acid segment encoding a polypeptide with ketoacyl-CoA synthase activity.

64. The method of claim 63, wherein the polypeptide with ketoacyl-CoA synthase activity is an NphT7 polypeptide.

65. The method of claim 63, wherein the polypeptide with ketoacyl-CoA synthase activity is an NphT7 variant selected from the group consisting of NphT7(I147S, F217V) and NphT7(H100L, I147S, F217V S323A).

66. The method of claim 63, wherein the polypeptide with ketoacyl-CoA synthase activity is a wild type Asch.

67. The method of claim 63, wherein the polypeptide with ketoacyl-CoA synthase activity is an Asch variant selected from the group consisting of Asch (T184I, F236L, V268A, V296A, V317A, S328G); Asch (T184I, V296A, V268A); and Asch (V296A).

68. The method of any one of claims 54-67, wherein the recombinant cell is a fungal cell or a bacterial cell.

69. The method of any one of claims 54-67, wherein the recombinant cell comprises an Escherichia coli.

70. The method of any one of claims 54-67, wherein the recombinant cell comprises a yeast cell.

71. A recombinant cell for producing a fatty acid ester, wherein the recombinant cell is genetically engineered for reduced activity of a polypeptide with at least 80%, 85%, 90%, 95%, 98%, or 100% sequence identity to an amino acid sequence set forth in SEQ ID NO: 4 and for reduced activity of a polypeptide with at least 80%, 85%, 90%, 95%, 98%, or 100% sequence identity to an amino acid sequence set forth in SEQ ID NO: 5,
wherein the recombinant cell is capable of producing at least 5 g/L total fatty acid ester.

72. The recombinant cell of claim 71, wherein the recombinant cell produces a reduction in total free fatty acids compared to a recombinant cell without reduced activity of a polypeptide with at least 80%, 85%, 90%, 95%, 98%, or 100% sequence identity to an amino acid sequence set forth in SEQ ID NO: 4 and for reduced activity of a polypeptide with at least 80%, 85%, 90%, 95%, 98%, or 100% sequence identity to an amino acid sequence set forth in SEQ ID NO: 5. [shake flask protocol]

73. The recombinant cell of claim 72, wherein the recombinant cell is genetically engineered for reduced activity of a polypeptide with at least 80%, 85%, 90%, 95%, 98%, or 100% sequence identity to an amino acid sequence set forth in SEQ ID NO: 6, and wherein the recombinant cell produces a reduction in total free fatty acids compared to a recombinant cell without reduced activity of a polypeptide with at least 80%, 85%, 90%, 95%, 98%, or 100% sequence identity to an amino acid sequence set forth in SEQ ID NO: 6.

74. The recombinant cell of any one of claims 72-73, wherein the reduction in free fatty acids comprises a reduction in C8 and/or C10 free fatty acids.

75. The recombinant cell of claim 71, wherein the recombinant cell produces a decreased ratio of total free fatty acids to total fatty acid esters compared to a recombinant cell without reduced activity of a polypeptide with at least 80%, 85%, 90%, 95%, 98%, or 100% sequence identity to an amino acid sequence set forth in SEQ ID NO: 4, without reduced activity of a polypeptide with at least 80%, 85%, 90%, 95%, 98%, or 100% sequence identity to an amino acid sequence set forth in SEQ ID NO: 5.

76. The recombinant cell of any one of claims 71-75, wherein the recombinant cell produces an increase in fatty acid ester of C8 chain length compared to a recombinant cell without reduced activity of a polypeptide with at least 80%, 85%, 90%, 95%, 98%, or 100% sequence identity to an amino acid sequence set forth in SEQ ID NO: 4, without reduced activity of a polypeptide with at least 80%, 85%, 90%, 95%, 98%, or 100% sequence identity to an amino acid sequence set forth in SEQ ID NO: 5, and/or without reduced activity of a polypeptide with at least 80%, 85%, 90%, 95%, 98%, or 100% sequence identity to an amino acid sequence set forth in SEQ ID NO: 6.

77. The recombinant cell of any one of claims 71-75, wherein the recombinant cell produces a fatty acid ester of C4, C6, C8, and/or C10 chain length.

78. The recombinant cell of any one of claims 71-75, wherein the recombinant cell produces a fatty acid ester of C8 and/or C10 chain length.

79. The recombinant cell of any one of claims 71-78, wherein the alkoxy group of the fatty acid ester is derived from a C1, C2, C3, or C4 monoalcohol.

80. The recombinant cell of claim 79, wherein the alkoxy group of the fatty acid ester is derived from a C1 or C2 monoalcohol.

81. The recombinant cell of claim 80, wherein the monoalcohol is methanol.

82. The recombinant cell of any one of claims 71-81, further comprising one or more nucleic acids selected from the group consisting of:
a heterologous nucleic acid segment encoding a polypeptide with enoyl-CoA reductase activity;
a heterologous nucleic acid segment encoding a polypeptide with bifunctional 3-hydroxyacyl-CoA dehydrogenase/dehydratase activity;
a heterologous nucleic acid segment encoding a polypeptide with ester synthase activity;
a heterologous nucleic acid segment encoding an enzyme with acetyl-CoA carboxylase activity; and
a heterologous nucleic acid segment encoding a polypeptide with ketoacyl-CoA synthase activity.

83. The recombinant cell of claim 82, wherein the polypeptide with ketoacyl-CoA synthase activity is an NphT7 polypeptide.

84. The recombinant cell of claim 82, wherein the polypeptide with ketoacyl-CoA synthase activity is an NphT7 variant selected from the group consisting of NphT7(I147S, F217V) and NphT7(H100L, I147S, F217V S323A).

85. The recombinant cell of claim 82, wherein the polypeptide with ketoacyl-CoA synthase activity is a wild type Asch.

86. The recombinant cell of claim 82, wherein the polypeptide with ketoacyl-CoA synthase activity is an Asch variant selected from the group consisting of Asch (T184I, F236L, V268A, V296A, V317A, S328G); Asch (T184I, V296A, V268A); and Asch (V296A).

87. The recombinant cell of any one of claims 71-86, wherein the recombinant cell is a fungal cell or a bacterial cell.

88. The recombinant cell of any one of claims 71-86, wherein the recombinant cell is an Escherichia coli.

89. The recombinant cell of any one of claims 71-86, wherein the recombinant cell is a yeast cell.

90. A method for decreasing free fatty acid production in a recombinant cell, the method comprising culturing a recombinant cell, wherein:
the recombinant cell is genetically engineered for reduced activity of a polypeptide with at least 80%, 85%, 90%, 95%, 98%, or 100% sequence identity to an amino acid sequence set forth in SEQ ID NO: 4 and for reduced activity of a polypeptide with at least 80%, 85%, 90%, 95%, 98%, or 100% sequence identity to an amino acid sequence set forth in SEQ ID NO: 5; and
the recombinant cell is capable of producing at least 5 g/L total fatty acid ester.

91. The method of claim 90, wherein the recombinant cell is genetically engineered for reduced activity of a polypeptide with at least 80%, 85%, 90%, 95%, 98%, or 100% sequence identity to an amino acid sequence set forth in SEQ ID NO: 6, and wherein the recombinant cell produces a reduction in total free fatty acids compared to a recombinant cell without reduced activity of a polypeptide with at least 80%, 85%, 90%, 95%, 98%, or 100% sequence identity to an amino acid sequence set forth in SEQ ID NO: 6.

92. The method of any one of claims 90-91, wherein the reduction in free fatty acids comprises a reduction in C8 and/or C10 free fatty acids.

93. The method of any one of claims 90-92, wherein the recombinant cell produces a decreased ratio of total free fatty acids to total fatty acid esters compared to a recombinant cell without reduced activity of a polypeptide with at least 80%, 85%, 90%, 95%, 98%, or 100% sequence identity to an amino acid sequence set forth in SEQ ID NO: 4 and/or without reduced activity of a polypeptide with at least 80%, 85%, 90%, 95%, 98%, or 100% sequence identity to an amino acid sequence set forth in SEQ ID NO: 5.

94. The method of any one of claims 90-93, wherein the recombinant cell produces an increase in fatty acid ester of C8 chain length compared to a recombinant cell without reduced activity of a polypeptide with at least 80%, 85%, 90%, 95%, 98%, or 100% sequence identity to an amino acid sequence set forth in SEQ ID NO: 4 and/or without reduced activity of a polypeptide with at least 80%, 85%, 90%, 95%, 98%, or 100% sequence identity to an amino acid sequence set forth in SEQ ID NO: 5.

95. The method of any one of claims 90-94, wherein the recombinant cell produces a fatty acid ester of C4, C6, C8, and/or C10 chain length.

96. The method of any one of claims 90-95, wherein the recombinant cell produces a fatty acid ester of C8 and/or C10 chain length.

97. The method of any one of claims 90-96, wherein the alkoxy group of the fatty acid ester is derived from a C1, C2, C3, or C4 monoalcohol.

98. The method of claim 97, wherein the alkoxy group of the fatty acid ester is derived from a C1 or C2 monoalcohol.

99. The method of claim 98, wherein the monoalcohol is methanol.

100. The method of any one of claims 90-99, further comprising one or more nucleic acids selected from the group consisting of:
a heterologous nucleic acid segment encoding a polypeptide with enoyl-CoA reductase activity;
a heterologous nucleic acid segment encoding a polypeptide with bifunctional 3-hydroxyacyl-CoA dehydrogenase/dehydratase activity;
a heterologous nucleic acid segment encoding a polypeptide with ester synthase activity;
a heterologous nucleic acid segment encoding an enzyme with acetyl-CoA carboxylase activity; and
a heterologous nucleic acid segment encoding a polypeptide with ketoacyl-CoA synthase activity.

101. The method of claim 100, wherein the polypeptide with ketoacyl-CoA synthase activity is an NphT7 polypeptide.

102. The method of claim 100, wherein the polypeptide with ketoacyl-CoA synthase activity is an NphT7 variant selected from the group consisting of NphT7(I147S, F217V) and NphT7(H100L, I147S, F217V S323A).

103. The method of claim 100, wherein the polypeptide with ketoacyl-CoA synthase activity is a wild type Asch.

104. The method of claim 100, wherein the polypeptide with ketoacyl-CoA synthase activity is an Asch variant selected from the group consisting of Asch (T184I, F236L, V268A, V296A, V317A, S328G); Asch (T184I, V296A, V268A); and Asch (V296A).

105. The method of any one of claims 90-104, wherein the recombinant cell is a fungal cell or a bacterial cell.

106. The method of any one of claims 90-104, wherein the recombinant cell is an Escherichia coli.

107. The method of any one of claims 90-104, wherein the recombinant cell comprises a yeast cell.

108. A recombinant cell for producing a fatty acid ester, wherein the recombinant cell is genetically engineered for increased activity of a polypeptide with at least 80%, 85%, 90%, 95%, 98%, or 100% sequence identity to an amino acid sequence set forth in SEQ ID NO: 7,
wherein the recombinant cell is capable of producing at least 5 g/L fatty acid ester.

109. The recombinant cell of claim 108, wherein the recombinant cell produces a reduction in total free fatty acids compared to a recombinant cell without reduced activity of a polypeptide with at least 80%, 85%, 90%, 95%, 98%, or 100% sequence identity to an amino acid sequence set forth in SEQ ID NO: 7 and for reduced activity of a polypeptide with at least 80%, 85%, 90%, 95%, 98%, or 100% sequence identity to an amino acid sequence set forth in SEQ ID NO: 7.

110. The recombinant cell of claim 109, wherein the reduction in free fatty acids comprises a reduction in C8 free fatty acids.

111. The recombinant cell of claim 108, wherein the recombinant cell produces a decreased ratio of total free fatty acids to total fatty acid esters compared to a recombinant cell without increased activity of a polypeptide with at least 80%, 85%, 90%, 95%, 98%, or 100% sequence identity to an amino acid sequence set forth in SEQ ID NO: 7.

112. The recombinant cell of any one of claims 108-111, wherein the recombinant cell produces a fatty acid ester of C4, C6, C8, and/or C10 chain length.

113. The recombinant cell of claim 112, wherein the recombinant cell produces a fatty acid ester of C8 and/or C10 chain length.

114. The recombinant cell of any one of claims 108-113, wherein the alkoxy group of the fatty acid ester is derived from a C1, C2, C3, or C4 monoalcohol.

115. The recombinant cell of claim 114, wherein the alkoxy group of the fatty acid ester is derived from a C1 or C2 monoalcohol.

116. The recombinant cell of claim 115, wherein the monoalcohol is methanol.

117. The recombinant cell of any of claims 108-116, further comprising one or more nucleic acids selected from the group consisting of:
a heterologous nucleic acid segment encoding a polypeptide with enoyl-CoA reductase activity;
a heterologous nucleic acid segment encoding a polypeptide with bifunctional 3-hydroxyacyl-CoA dehydrogenase/dehydratase activity;
a heterologous nucleic acid segment encoding a polypeptide with ester synthase activity;
a heterologous nucleic acid segment encoding an enzyme with acetyl-CoA carboxylase activity; and
a heterologous nucleic acid segment encoding a polypeptide with ketoacyl-CoA synthase activity.

118. The recombinant cell of claim 117, wherein the polypeptide with ketoacyl-CoA synthase activity is an NphT7 polypeptide.

119. The recombinant cell of claim 117, wherein the polypeptide with ketoacyl-CoA synthase activity is an NphT7 variant selected from the group consisting of NphT7(I147S, F217V) and NphT7(H100L, I147S, F217V S323A).

120. The recombinant cell of claim 117, wherein the polypeptide with ketoacyl-CoA synthase activity is a wild type Asch.

121. The recombinant cell of claim 117, wherein the polypeptide with ketoacyl-CoA synthase activity is an Asch variant selected from the group consisting of Asch (T184I, F236L, V268A, V296A, V317A, S328G); Asch (T184I, V296A, V268A); and Asch (V296A).

122. The recombinant cell of any one of claims 108-121, wherein the recombinant cell is a fungal cell or a bacterial cell.

123. The recombinant cell of any one of claims 108-121, wherein the recombinant cell is an Escherichia coli.

124. The recombinant cell of any one of claims 108-121, wherein the recombinant cell is a yeast cell.

125. The recombinant cell of any one of claims 108-124, wherein increased activity of a polypeptide with at least 80%, 85%, 90%, 95%, 98%, or 100% sequence identity to an amino acid sequence set forth in SEQ ID NO: 7 comprises inducible expression of the gene encoding the polypeptide.

126. The recombinant cell of any one of claims 108-124, wherein increased activity of a polypeptide with at least 80%, 85%, 90%, 95%, 98%, or 100% sequence identity to an amino acid sequence set forth in SEQ ID NO: 7 comprises constitutive expression of the gene encoding the polypeptide.

127. A method for decreasing free fatty acid production, the method comprising culturing a recombinant cell, wherein:
the recombinant cell is genetically engineered for increased activity of a polypeptide with at least 80%, 85%, 90%, 95%, 98%, or 100% sequence identity to an amino acid sequence set forth in SEQ ID NO: 7;
wherein the recombinant cell produces a reduction in total free fatty acids compared to a recombinant cell without increased activity of a polypeptide with at least 80%, 85%, 90%, 95%, 98%, or 100% sequence identity to an amino acid sequence set forth in SEQ ID NO: 7; and
the recombinant cell is capable of producing at least 5 g/L total fatty acid ester.

128. The method of claim 127, wherein the reduction in free fatty acids comprises a reduction in C8 free fatty acids.

129. The method of claim 127, wherein the recombinant cell produces a decreased ratio of total free fatty acids to total fatty acid esters compared to a recombinant cell without reduced activity of a polypeptide with at least 80%, 85%, 90%, 95%, 98%, or 100% sequence identity to an amino acid sequence set forth in SEQ ID NO: 7.

130. The method of any one of claims 127-129, wherein the recombinant cell produces a fatty acid ester of C4, C6, C8, and/or C10 chain length.

131. The method of any one of claims 127-129, wherein the recombinant cell produces a fatty acid ester of C8 and/or C10 chain length.

132. The method of any one of claims 127-131, wherein the alkoxy group of the fatty acid ester is derived from a C1, C2, C3, or C4 monoalcohol.

133. The method of claim 132, wherein the alkoxy group of the fatty acid ester is derived from a C1 or C2 monoalcohol.

134. The method of claim 133, wherein the monoalcohol is methanol.

135. The method of any of claims 127-134, wherein the recombinant cell further comprises one or more nucleic acids selected from the group consisting of:
a heterologous nucleic acid segment encoding a polypeptide with enoyl-CoA reductase activity;
a heterologous nucleic acid segment encoding a polypeptide with bifunctional 3-hydroxyacyl-CoA dehydrogenase/dehydratase activity;
a heterologous nucleic acid segment encoding a polypeptide with ester synthase activity;
a heterologous nucleic acid segment encoding an enzyme with acetyl-CoA carboxylase activity; and
a heterologous nucleic acid segment encoding a polypeptide with ketoacyl-CoA synthase activity.

136. The method of claim 135, wherein the polypeptide with ketoacyl-CoA synthase activity is an NphT7 polypeptide.

137. The method of claim 135, wherein the polypeptide with ketoacyl-CoA synthase activity is an NphT7 variant selected from the group consisting of NphT7(I147S, F217V) and NphT7(H100L, I147S, F217V, S323A).

138. The method of claim 135, wherein the polypeptide with ketoacyl-CoA synthase activity is a wild type Asch.

139. The method of claim 135, wherein the polypeptide with ketoacyl-CoA synthase activity is an Asch variant selected from the group consisting of Asch (T184I, F236L, V268A, V296A, V317A, S328G); Asch (T184I, V296A, V268A); and Asch (V296A).

140. The method of any one of the claims 127-139, wherein the recombinant cell is a fungal cell or a bacterial cell.

141. The method of any one of claims 127-139, wherein the recombinant cell is an Escherichia coli.

142. The method of any one of claims 127-139, wherein the recombinant cell comprises a yeast cell.

143. The method of any one of claims 127-142, wherein increased activity of a polypeptide with at least 80%, 85%, 90%, 95%, 98%, or 100% sequence identity to an amino acid sequence set forth in SEQ ID NO: 7 comprises inducible expression of the gene.

144. The method of any one of claims 127-142, wherein increased activity of a polypeptide with at least 80%, 85%, 90%, 95%, 98%, or 100% sequence identity to an amino acid sequence set forth in SEQ ID NO: 7 comprises constitutive expression of the gene.

145. A recombinant cell for producing a fatty acid ester, wherein the recombinant cell is genetically engineered for increased activity of a polypeptide with at least 80%, 85%, 90%, 95%, 98%, or 100% sequence identity to an amino acid sequence set forth in SEQ ID NO: 7,
wherein the recombinant cell is genetically engineered for reduced activity of a polypeptide with at least 80%, 85%, 90%, 95%, 98%, or 100% sequence identity to an amino acid sequence set forth in SEQ ID NO: 4 and for reduced activity of a polypeptide with at least 80%, 85%, 90%, 95%, 98%, or 100% sequence identity to an amino acid sequence set forth in SEQ ID NO: 5,
wherein the recombinant cell is capable of producing at least 5 g/L total fatty acid ester.

146. The recombinant cell of claim 145, wherein the reduction in free fatty acids comprises a reduction in C8 and/or C10 free fatty acids.

147. The recombinant cell of claim 145, wherein the recombinant cell produces increased C8 fatty acid esters compared to a recombinant cell without reduced activity of a polypeptide with at least 80%, 85%, 90%, 95%, 98%, or 100% sequence identity to an amino acid sequence set forth in SEQ ID NO: 4 and/or for reduced activity of a polypeptide with at least 80%, 85%, 90%, 95%, 98%, or 100% sequence identity to an amino acid sequence set forth in SEQ ID NO: 5.

148. The recombinant cell of any one of claims 145-147, wherein the recombinant cell produces a fatty acid ester of C4, C6, C8, and/or C10 chain length.

149. The recombinant cell of claim 148, wherein the recombinant cell produces a fatty acid ester of C8 and/or C10 chain length.

150. The recombinant cell of any one of claims 145-149, wherein the alkoxy group of the fatty acid ester is derived from a C1, C2, C3, or C4 monoalcohol.

151. The recombinant cell of claim 150, wherein the alkoxy group of the fatty acid ester is derived from a C1 or C2 monoalcohol.

152. The recombinant cell of claim 151, wherein the monoalcohol is methanol.

153. The recombinant cell of any of claims 145-152, further comprising one or more nucleic acids selected from the group consisting of:
a heterologous nucleic acid segment encoding a polypeptide with enoyl-CoA reductase activity;
a heterologous nucleic acid segment encoding a polypeptide with bifunctional 3-hydroxyacyl-CoA dehydrogenase/dehydratase activity;
a heterologous nucleic acid segment encoding a polypeptide with ester synthase activity;
a heterologous nucleic acid segment encoding an enzyme with acetyl-CoA carboxylase activity; and
a heterologous nucleic acid segment encoding a polypeptide with ketoacyl-CoA synthase activity.

154. The recombinant cell of claim 153, wherein the polypeptide with ketoacyl-CoA synthase activity is an NphT7 polypeptide.

155. The recombinant cell of claim 153, wherein the polypeptide with ketoacyl-CoA synthase activity is an NphT7 variant selected from the group consisting of NphT7(I147S, F217V) and NphT7(H100L, I147S, F217V, S323A).

156. The recombinant cell of claim 153, wherein the polypeptide with ketoacyl-CoA synthase activity is a wild type Asch.

157. The recombinant cell of claim 153, wherein the polypeptide with ketoacyl-CoA synthase activity is an Asch variant selected from the group consisting of Asch (T184I, F236L, V268A, V296A, V317A, S328G); Asch (T184I, V296A, V268A); and Asch (V296A).

158. The recombinant cell of any one of claims 145-157, wherein the recombinant cell is a fungal cell or a bacterial cell.

159. The recombinant cell of any one of claims 145-157, wherein the recombinant cell is an Escherichia coli.

160. The recombinant cell of any one of claims 145-157, wherein the recombinant cell is a yeast cell.

161. The recombinant cell of any one of claims 145-160, wherein increased activity of a polypeptide with at least 80%, 85%, 90%, 95%, 98%, or 100% sequence identity to an amino acid sequence set forth in SEQ ID NO: 7 comprises inducible expression of the gene encoding the polypeptide.

162. The recombinant cell of any one of claims 145-160, wherein increased activity of a polypeptide with at least 80%, 85%, 90%, 95%, 98%, or 100% sequence identity to an amino acid sequence set forth in SEQ ID NO: 7 comprises constitutive expression of the gene encoding the polypeptide.

163. A method for decreasing free fatty acid production, the method comprising culturing a recombinant cell, wherein:
the recombinant cell is genetically engineered for increased activity of a polypeptide with at least 80%, 85%, 90%, 95%, 98%, or 100% sequence identity to an amino acid sequence set forth in SEQ ID NO: 7;
the recombinant cell is genetically engineered for reduced activity of a polypeptide with at least 80%, 85%, 90%, 95%, 98%, or 100% sequence identity to an amino acid sequence set forth in SEQ ID NO: 4 and for reduced activity of a polypeptide with at least 80%, 85%, 90%, 95%, 98%, or 100% sequence identity to an amino acid sequence set forth in SEQ ID NO: 5; and
the recombinant cell is capable of producing at least 5 g/L of total fatty acid ester.

164. The method of claim 163, wherein the reduction in free fatty acids comprises a reduction in C8 and/or C10 free fatty acids.

165. The method of claim 163, wherein the recombinant cell produces increased C8 fatty acid esters compared to a recombinant cell without reduced activity of a polypeptide with at least 80%, 85%, 90%, 95%, 98%, or 100% sequence identity to an amino acid sequence set forth in SEQ ID NO: 4 and/or for reduced activity of a polypeptide with at least 80%, 85%, 90%, 95%, 98%, or 100% sequence identity to an amino acid sequence set forth in SEQ ID NO: 5.

166. The method of any one of claims 163-165, wherein the recombinant cell produces a fatty acid ester of C4, C6, C8, and/or C10 chain length.

167. The method of claim 166, wherein the recombinant cell produces a fatty acid ester of C8 and/or C10 chain length.

168. The method of any one of claims 163-167, wherein the alkoxy group of the fatty acid ester is derived from a C1, C2, C3, or C4 monoalcohol.

169. The method of claim 168, wherein the alkoxy group of the fatty acid ester is derived from a C1 or C2 monoalcohol.

170. The method of claim 169, wherein the monoalcohol is methanol.

171. The method of any of claims 163-170, wherein the recombinant cell further comprises one or more nucleic acids selected from the group consisting of:
a heterologous nucleic acid segment encoding a polypeptide with enoyl-CoA reductase activity;
a heterologous nucleic acid segment encoding a polypeptide with bifunctional 3-hydroxyacyl-CoA dehydrogenase/dehydratase activity;
a heterologous nucleic acid segment encoding a polypeptide with ester synthase activity;
a heterologous nucleic acid segment encoding an enzyme with acetyl-CoA carboxylase activity; and
a heterologous nucleic acid segment encoding a polypeptide with ketoacyl-CoA synthase activity.

172. The method of claim 171, wherein the polypeptide with ketoacyl-CoA synthase activity is an NphT7 polypeptide.

173. The method of claim 171, wherein the polypeptide with ketoacyl-CoA synthase activity is an NphT7 variant selected from the group consisting of NphT7(I147S, F217V) and NphT7(H100L, I147S, F217V, S323A).

174. The method of claim 171, wherein the polypeptide with ketoacyl-CoA synthase activity is a wild type Asch.

175. The method of claim 171, wherein the polypeptide with ketoacyl-CoA synthase activity is an Asch variant selected from the group consisting of Asch (T184I, F236L, V268A, V296A, V317A, S328G); Asch (T184I, V296A, V268A); and Asch (V296A).

176. The method of any one of claims 163-175, wherein the recombinant cell is a fungal cell, a bacterial cell, or a plant cell.

177. The method of any one of claims 163-175, wherein the recombinant cell comprises an Escherichia coli.

178. The method of any one of claims 163-175, wherein the recombinant cell comprises a yeast cell.

179. The method of any one of claims 163-178, wherein increased activity of a polypeptide with at least 80%, 85%, 90%, 95%, 98%, or 100% sequence identity to an amino acid sequence set forth in SEQ ID NO: 7 comprises inducible expression of the gene encoding the polypeptide.

180. The method of any one of claims 163-178, wherein increased activity of a polypeptide with at least 80%, 85%, 90%, 95%, 98%, or 100% sequence identity to an amino acid sequence set forth in SEQ ID NO: 7 comprises constitutive expression of the gene encoding the polypeptide.
